(19) United States
(12) Patent Application Publication
(10) Pub. No.: US 2023/0363713 A1
(43) Pub. Date: Nov. 16, 2023

(54) HYDROGEL MICRONEEDLES FOR BIOSENSING

(71) Applicants: MAHLA POUDINEH, WATERLOO (CA); AMIN GHAVAMI NEJAD, TORONTO (CA); HANJIA ZHENG, WATERLOO (CA); FATEMEH KEYVANI, WATERLOO (CA)

(72) Inventors: MAHLA POUDINEH, WATERLOO (CA); AMIN GHAVAMI NEJAD, TORONTO (CA); HANJIA ZHENG, WATERLOO (CA); FATEMEH KEYVANI, WATERLOO (CA)

(21) Appl. No.: 17/962,055

(22) Filed: Oct. 7, 2022

Related U.S. Application Data
(60) Provisional application No. 63/253,781, filed on Oct. 8, 2021.

(51) Int. Cl. A61B 5/00; A61B 5/145

(52) U.S. Cl. CPC A61B 5/685; A61B 5/14514

(57) ABSTRACT

Microneedles for detecting targets are described. The microneedles may be made of a hydrogel and a probe coupled to the hydrogel for generating a measurable signal in the presence of the target. The hydrogel microneedles may be used for in-situ detection of targets, such as biomolecules found in interstitial fluid. Also described are methods or producing hydrogel microneedles, articles and apparatus comprising hydrogel microneedles, and methods and uses of the same. The hydrogel microneedles may be used for biosensing, such as in transdermal patches for detecting biomarkers in a subject. The biosensors may be used for continuous, real-time tracking of targets in-situ, without requiring further reagents or processing steps.

### CROSS REFERENCE TO RELATED APPLICATION

This application claims priority to U.S. Provisional Patent Application No. 63/253,781 filed Oct. 8, 2021, the entire contents of which are hereby incorporated by reference.

### SEQUENCE LISTING

The instant application contains a Sequence Listing which has been filed electronically in XML format and is hereby incorporated by reference in its entirety. Said XML, created on Apr. 21, 2023, is named file:///C:/Users/wslade/AppData/Local/Microsoft/Windoes/INetCache/Content.Outlook/3SRO 6ESJ/GBP-Rluc-updated%20ST26_%202023_Jin%20Zhang%20(002).xml and is 36 KB in size.

### FIELD

The present disclosure relates generally to hydrogel microneedles, their methods of production, and methods and uses thereof in biosensing.

### BACKGROUND

Transdermal biosensing can bring us one step closer to personalized and precision medicine, as it enables the continuous tracking of patient health conditions in a non- or minimally invasive manner². Transdermal biosensors analyze interstitial fluid (ISF), the fluid which is present in the lowermost skin layer of the dermis, for biomarker measurements^{2,4}. Compared to other body fluids, ISF has the most similar molecular composition to blood plasma⁵, in addition to possessing other unique features including biomarkers of medical relevance². Simple and effective methods that enable the comprehensive analysis of ISF can lead to transformative advances in bio-diagnostic technologies. These approaches are not only minimally invasive and painless, but also ideally suited for point-of-care and resource-limited settings.

Microneedle (MN)-based techniques have been introduced as effective approaches for simple ISF extraction with the potential of integrating diagnostics. Different types of MNs implement various strategies to obtain ISF, for example, hollow MNs operate based on negative pressure; porous MNs use capillary force; and the most recent one, hydrogel-based MNs (HMNs) employ material absorption property. HMNs with a length less than 1000 pm and tips much sharper than hypodermic needle enable efficient piercing of the stratum corneum (outer layer of the skin) and the formation of microscale ISF extraction channels^{4,16}. Compared to other MNs, HMNs possess several advantages, including increased and rapid ISF extraction, high biocompatibility, lower fabrication cost, higher production yield, and most importantly ease of insertion and removal without causing skin damage^{4,16,18}.

Integrating biosensors on MNs enables in situ ISF characterization⁴. Hollow, metallic MN devices combined with enzymes have been implemented for real-time monitoring of various metabolites, electrolytes, and therapeutics. The main obstacles with hollow MN applications are the complex fabrication protocols and the potential risk of MN clogging. A solid hydrophobic microneedle functionalized with antibodies has been recently reported for the specific capture of target biomarkers in ISF, followed by ex vivo analysis²⁰. Although MNs functionalized with antibodies allow for on-needle biomarker detection in ISF, the sensor still needs post-processing steps, such as washing and adding detection reagents to detect targets of interest.

### SUMMARY

In one aspect there is provided a microneedle for detecting a target, the microneedle comprising: a hydrogel; a probe coupled to the hydrogel, the probe for generating a measurable signal in the presence of the target.

In one embodiment there is provided a microneedle, wherein the measurable signal is generated in situ.

In one embodiment there is provided a microneedle, wherein the hydrogel comprises a polymer comprising at least one C═C functionality.

In one embodiment there is provided a microneedle, wherein the hydrogel comprises an acrylated polymer, a methacrylated polymer, or a combination thereof.

In one embodiment there is provided a microneedle, wherein the hydrogel comprises methacrylated gelatin, methacrylated hyaluronic acid, methacrylated alginate, methacrylated chitosan, methacrylated collagen, or a combination thereof.

In one embodiment there is provided a microneedle, wherein the hydrogel comprises functionalized hyaluronic acid.

In one embodiment there is provided a microneedle, wherein the hydrogel comprises methacrylated hyaluronic acid.

In one embodiment there is provided a microneedle, wherein the probe coupled to the hydrogel comprises the probe being coupled to the hydrogel by a linker.

In one embodiment there is provided a microneedle, wherein the linker is formed from a phosphoramidite functional group, such as an acrydite functional group.

In one embodiment there is provided a microneedle, wherein the probe coupled to the hydrogel comprises the probe being covalently bonded to the hydrogel.

In one embodiment there is provided a microneedle, wherein the hydrogel further comprises a nanoparticle.

In one embodiment there is provided a microneedle, wherein the probe coupled to the hydrogel comprises the probe being coupled to the nanoparticle.

In one embodiment there is provided a microneedle, wherein the probe comprises an aptamer.

In one embodiment there is provided a microneedle, wherein the aptamer is an aptamer that binds to the target.

In one embodiment there is provided a microneedle, wherein the aptamer comprises a sequence according to a SEQ ID No. herein disclosed.

In one embodiment there is provided a microneedle, wherein the aptamer comprises a linker functional group for coupling the probe to the hydrogel.

In one embodiment there is provided a microneedle, wherein the linker functional group comprises a phosphoramidite functional group.

In one embodiment there is provided a microneedle, wherein the linker functional group comprises an acrydite functional group.

In one embodiment there is provided a microneedle, wherein the probe comprises a fluorophore.

In one embodiment there is provided a microneedle, wherein the probe comprises an aptamer, and the aptamer comprises a fluorophore or is linked to a fluorophore.

In one embodiment there is provided a microneedle, wherein the probe is reversibly bound to a quencher.

In one embodiment there is provided a microneedle, wherein the probe is an aptamer and the quencher comprises a sequence partially or fully complimentary to at least a portion of the aptamer sequence.

In one embodiment there is provided a microneedle, wherein the quencher comprises a sequence according to a SEQ ID No. herein disclosed.

In one embodiment there is provided a microneedle, wherein the measurable signal is fluorescence.

In one embodiment there is provided a microneedle, wherein the target is a biomolecule present in interstitial fluid.

In one embodiment there is provided a microneedle, wherein the target is adenosine triphosphate.

In one embodiment there is provided a microneedle, wherein the target is glucose.

In one embodiment there is provided a microneedle, wherein the microneedle has a length of about 300 μm to about 1000 μm, such as about 800 μm.

In another aspect there is provided a method of producing a microneedle, the method comprising: combining a functionalized hydrogel, a probe precursor, and a crosslinking agent in a mold; and exposing the mixture in the mold to UV light to link at least a portion of the probe to the functionalized hydrogel and to form a crosslinked material.

In one embodiment there is provided a method further comprising functionalizing a hydrogel to form the functionalized hydrogel.

In one embodiment there is provided a method, further comprising: removing the crosslinked material from the mold; and further exposing the unmolded crosslinked material to UV light.

In one embodiment there is provided a method, further comprising washing the crosslinked material to remove unbound probes.

In one embodiment there is provided a method, wherein combining the functionalized hydrogel, the probe precursor, and the crosslinking agent in the mold comprises: dissolving about 50:1 to about 10:1 (wt/wt) of functionalized hydrogel:crosslinking agent in a buffer to form a functionalized hydrogel solution; optionally degassing the functionalized hydrogel solution; adding the functionalized hydrogel solution to the mold; partially drying the functionalized hydrogel solution in the mold; optionally, adding further functionalized hydrogel solution to the mold; adding the probe precursor to the mold; and optionally, drying the mixture in the mold further.

In one embodiment there is provided a method, wherein the probe precursor is a solution of aptamer and quencher, preferably in a ratio of about 1:5 to about 1:20, such as a 1:10 ratio.

In one embodiment there is provided a method, wherein exposing the mixture in the mold to UV light comprises exposing the mixture to light of about 200 nm to about 400 nm, preferably about 360 nm light; and preferably for about 1 min to about 1 hour, such as about 10 to about 20 min.

In one embodiment there is provided a method, wherein the hydrogel comprises hyaluronic acid.

In one embodiment there is provided a method, wherein functionalizing the hydrogel comprises reacting hyaluronic acid with methacrylic anhydride to form methacrylated hyaluronic acid.

In one embodiment there is provided a method, wherein combining the functionalized hydrogel, the probe precursor and the crosslinking agent in the mold further comprises adding a photoinitiator to the mixture in the mold.

In one embodiment there is provided a method, wherein the crosslinking agent comprises N,N′-methylenebisacrylamide.

In one embodiment there is provided a method, wherein the mold is a negative polydimethylsiloxane mold.

In another aspect there is provided a microneedle obtainable or obtained by a method herein disclosed.

In another aspect there is provided an apparatus for detecting a target in a sample, the apparatus comprising: the microneedle according to an embodiment herein disclosed; and a detector for detecting the measurable signal.

In one embodiment there is provided as apparatus, wherein the detector is a fluorimeter.

In another aspect there is provided a transdermal patch comprising the microneedle according to an embodiment herein disclosed.

In another aspect there is provided a method for transdermal biosensing of a target in a subject, the method comprising: applying a transdermal patch according to an embodiment herein disclosed; detecting the measurable signal; and associating the measurable signal to the concentration of the target in the subject.

In one embodiment there is provided a method, wherein detecting the measurable signal is in situ.

In one embodiment there is provided a method, wherein detecting the measurable signal is reagentless.

In one embodiment there is provided a method, wherein detecting the measurable signal occurs without requiring removal of the transdermal patch.

In one embodiment there is provided a method, wherein detecting the measurable signal occurs while the transdermal patch is applied to the subject.

In one embodiment there is provided a method, wherein detecting the measurable signal occurs in the absence of further processing of the transdermal patch.

In one embodiment there is provided a method, wherein detecting the measurable signal comprises measuring the fluorescence intensity of the probe.

In one embodiment there is provided a method, wherein associating the measurable signal comprises comparing a measured intensity of the measurable signal to a calibration curve of measured intensities of known concentrations of the target.

In an aspect of the present disclosure, there is provided a microneedle for detecting a target, the microneedle comprising: a hydrogel; a probe coupled to the hydrogel, the probe for generating a measurable signal in the presence of the target.

In an embodiment of the present disclosure, there is provided a microneedle wherein the hydrogel comprises a polymer comprising at least one C═C functionality; an acrylated polymer, a methacrylated polymer, or a combination thereof; and/or methacrylated gelatin, methacrylated hyaluronic acid, methacrylated alginate, methacrylated chitosan, methacrylated collagen, methacrylated polyethylene glycol, methacrylated polyvinyl alcohol, methacrylated polylysine, or a combination thereof.

In another embodiment, there is provided a microneedle wherein the hydrogel further comprises a conductive polymer, an ionomer, or a combination thereof; or poly(3,4-ethylenedioxythiophene) polystyrene sulfonate; polyacetylene; polypyrrole; polyindole; polyaniline; a copolymer thereof; or a combination thereof.

In another embodiment, there is provided a microneedle wherein the probe coupled to the hydrogel comprises the probe being coupled to the hydrogel by covalent bonding, intermolecular bonding, physisorption, complexation, a linker; or a combination thereof.

In another embodiment, there is provided a microneedle wherein the probe comprises a nucleic acid, wherein the nucleic acid is an nucleic acid that binds to the target; and wherein the nucleic acid optionally comprises an aptamer, single stranded complementary probe DNA, peptide nucleic acid, nucleic acid enzyme, or combinations thereof.

In another embodiment, there is provided a microneedle wherein the nucleic acid comprises a linker functional group for coupling the probe to the hydrogel. In an embodiment, the linker functional group comprises a phosphoramidite functional group; or an acrydite functional group.

In another embodiment, there is provided a microneedle wherein the probe comprises a fluorophore, or an electroactive species, a redox active species, or a combination thereof, such as a redox reporter. In an embodiment, the probe comprises a nucleic acid, and the nucleic acid comprises a fluorophore or is linked to a fluorophore, or the nucleic acid comprises a redox reporter or is linked to a redox reporter. In an embodiment, the redox reporter comprises methylene blue, ferrocene, or a combination thereof.

In another embodiment, there is provided a microneedle wherein the probe further comprises a quencher, and the probe is optionally reversibly bound to a quencher, or is optionally tethered to the quencher via covalent bonding, intermolecular bonding, physical adsorption, conjugation, or a combination thereof.

In another embodiment, there is provided a microneedle wherein the probe comprises a nucleic acid and the quencher comprises a sequence partially or fully complimentary to at least a portion of the nucleic acid sequence.

In another embodiment, there is provided a microneedle wherein the probe comprises a nucleic acid and the quencher comprises a graphene-based material, wherein the graphene-based material optionally comprises graphene-oxide (GO) nanosheets, graphene-oxide (GO) nanoparticles, graphene-oxide (GO) nanocomposites, or a combination thereof.

In another embodiment, there is provided a microneedle wherein the probe comprises a nucleic acid and the nucleic acid comprises a linker functional group for coupling the probe to the quencher.

In another embodiment, there is provided a microneedle wherein the measurable signal is fluorescence; or an electrochemical signal.

In another embodiment, there is provided a microneedle wherein the target comprises a biomolecule present in interstitial fluid. In an embodiment, the target comprises small biomolecules, proteins, or micro ribonucleic acids; or cortisol, vanomycin, gentamicin, tyrosinamide, thrombin, micro-RNA miR21, micro-RNA miR210, uric acid (UA), serotonin, insulin, adenosine triphosphate, or glucose.

In another embodiment, there is provided a microneedle wherein the microneedle has a length of about 300 μm to about 1000 μm, such as about 800 μm.

In another embodiment, there is provided a microneedle further comprising a conductive material, wherein the conductive material optionally comprises a metal nanoparticle, graphene-based material, conductive polymer, or an ionomer. In an embodiment, the conductive polymer or ionomer comprises poly(3,4-ethylenedioxythiophene) polystyrene sulfonate; polyacetylene; polypyrrole; polyindole; polyaniline; a copolymer thereof; or a combination thereof. In an embodiment, the graphene-based material comprises ferrocene, graphene-oxide (GO) nanosheets, graphene-oxide (GO) nanoparticles, graphene-oxide (GO) nanocomposites, or a combination thereof.

In another aspect, there is provided a method of producing a microneedle, the method comprising: combining a functionalized hydrogel, a probe precursor, optionally a conductive material, and a crosslinking agent in a mold; and exposing the mixture in the mold to UV light to link at least a portion of the probe to the functionalized hydrogel and to form a crosslinked material.

In another embodiment, there is provided a method further comprising removing the crosslinked material from the mold; and further exposing the unmolded crosslinked material to UV light.

In another embodiment, there is provided a method wherein combining the functionalized hydrogel, the probe precursor, optionally a conductive material, and the crosslinking agent in the mold comprises: dissolving about 50:1 to about 10:1 (wt/wt) of functionalized hydrogel:crosslinking agent in a buffer to form a functionalized hydrogel solution; optionally adding a conductive material to the functionalized hydrogel solution; optionally degassing the functionalized hydrogel solution; adding the functionalized hydrogel solution to the mold; partially drying the functionalized hydrogel solution in the mold; optionally, adding further functionalized hydrogel solution to the mold; adding the probe precursor to the mold; and optionally, drying the mixture in the mold further.

In another embodiment, there is provided a method wherein the probe precursor comprises a solution of nucleic acid and optionally a quencher.

In another embodiment, there is provided a method wherein exposing the mixture in the mold to UV light comprises exposing the mixture to light of about 200 nm to about 400 nm, preferably about 360 nm light; and preferably for about 1 min to about 1 hour, such as about 10 to about 20 min.

In another embodiment, there is provided a method wherein the hydrogel comprises hyaluronic acid; and functionalizing functionalizing the hydrogel comprises reacting hyaluronic acid with methacrylic anhydride to form methacrylated hyaluronic acid.

In another embodiment, there is provided a method wherein the optional conductive material comprises metal nanoparticles, graphene-based material, or a conductive polymer or ionomer, such as poly(3,4-ethylenedioxythiophene) polystyrene sulfonate, polyacetylene, polypyrrole, polyindole, polyaniline, or copolymers thereof.

In another embodiment, there is provided a method wherein the mold is a negative polydimethylsiloxane mold.

In another aspect, there is provided an apparatus for detecting a target in a sample, the apparatus comprising: the microneedle as described herein; and a detector for detecting the measurable signal.

In another aspect, there is provided a transdermal patch comprising the microneedle described herein.

In another aspect, there is provided a method for transdermal biosensing of a target in a subject, the method comprising: applying the transdermal patch described herein; detecting the measurable signal; and associating the measurable signal to the concentration of the target in the subject.

In an embodiment, there is provided a method wherein detecting the measurable signal is reagentless. In an embodiment, there is provided a method wherein detecting the measurable signal comprises measuring the fluorescence intensity of the probe; or measuring an electrochemical signal. In an embodiment, there is provided a method wherein associating the measurable signal comprises comparing a measured intensity of the measurable signal to a calibration curve of measured intensities of known concentrations of the target.

### BRIEF DESCRIPTION OF THE FIGURES

Embodiments of the present disclosure will now be described, by way of example only, with reference to the attached Figures.

FIG. 1 depicts a microneedle according to an embodiment of the present disclosure.

FIGS. 2A-2C depict an overview of a reagentless fluorescence assay for minimally invasive detection (RFMID) functionalization and sensing strategy.

FIGS. 3A-3C depict a RFMID fabrication and functionalization.

FIGS. 4A-4D depict a RFMID characterization.

FIGS. 5A-5H depict in vitro and ex vivo detection of ATP and glucose.

FIGS. 6A-6C depict in vivo glucose detection in diabetic rats.

FIG. 7 depicts ¹H NMR spectra of a MeHA.

FIG. 8 depicts characterization of a MeHA-HMN swelling ability.

FIG. 9 depicts characterization of a MeHA-HMN recovery rate.

FIGS. 10A and 10B depict an assessment of aptamer binding efficiency.

FIG. 11 depicts in vitro porcine skin penetration efficiency test using a RFMID.

FIG. 12 depicts a biocompatibility test of an aptamer MeHA-HMN.

FIG. 13 depicts glucose measurement in live diabetic animals using a RFMID.

FIGS. 14A-14D depict HMN-GO.NA sensing strategy and workflow of HMN-GO.NA assay.

FIGS. 15A-15G depict HMN-GO.NA assay fabrication and characterization.

FIGS. 16A-16I depict ex-vivo validation of HMN-GO.NA for biomarker detection.

FIGS. 17A-17D depict a portable and smartphone-based detector for POCT setting.

FIGS. 18A-18E depict in-vivo UA and glucose detection in an animal model of diabetes.

FIGS. 19A-19G depict In vitro characterization of RFMID using an agarose hydrogel model.

FIGS. 20A-20F depict ex vivo characterization of RFMID using a porcine skin model.

FIGS. 21A-21D depict Assessment of aptamer binding efficiency.

FIG. 22 depicts the cross-reactivity of RFMID device for glucose capture in the presence of common interfering agents.

FIG. 23 depicts stability test of RFMID patch for glucose measurement.

FIG. 24 depicts HMN patches were applied on agarose hydrogel for different durations (0, 30 s, 60 s, 90 s, 120 s, and 300 s) and the changed in the needle volume was observed on the microscope.

FIGS. 25A and 25B depict in-vitro ATP detection to establish proof of concept.

FIGS. 26A-26F depict in-vitro validation of HMN-GO.NA patch.

FIGS. 27A and 27B depict histology imaging analysis of (A) a control rat's skin (scale bar=2400 μm) and (B) rat's skin one hour after HMN-GO.NA removal (scale bar=2000 μm).

### DETAILED DESCRIPTION

Analyzing interstitial fluid (ISF) via microneedle (MN) devices can enable patient health monitoring in a minimally invasive manner and at point-of-care settings. However, most MN-based diagnostic approaches require complicated fabrication processes or post-processing of the extracted ISF. Described herein is an in situ and on-needle measurement of target analytes performed by integrating hydrogel microneedles (HMN) with nucleic acid probes (e.g., aptamer probes, pDNA probes) as the target recognition elements.

In one example, fluorescently tagged aptamer probes are chemically attached to a hydrogel matrix while a crosslinked patch is formed. In another example, fluorophore-modified nucleic acid (e.g., apatmers, probe DNA) are immobilized on or tethered to a quencher (e.g., graphene-based quencher), and the probe (comprising the nucleic acid and further comprising the quencher) is coupled to a hydrogel matrix via intermolecular interactions, such as hydrogen bonding.

In another example, the HMNs described herein are capable of continuous electrochemical measurement via integration with a probe that comprises a redox reporter, and a conductive material that communicates electrochemical signals through the hydrogel. In an example, the conductive material comprises conductive polymer poly(3,4-ethylenedioxythiophene) polystyrene sulfonate (PEDOT:PSS)—which, when integrated into a hydrogel network, can boost the electrical properties of the HMN, making the HMN suitable for use as a working electrode.

This system may improve the quality of life of patients who are in need of close monitoring of biomarkers of health and disease. The HMNs described herein can enable rapid and reagentless target detection of targets such as glucose, ATP, uric acid, serotonin, insulin, cortisol, vanomycin, gentamicin, tyrosinamide, thrombin, micro-RNAs such as miR21 and miR210.

In an example, to demonstrate the effectiveness of such a system, an assay may be used for specific and sensitive quantification of glucose concentrations in an animal model of diabetes to track hypoglycemia, euglycemia, and hyperglycemia conditions. The assay may track the rising and falling concentrations of glucose and the extracted measurements closely match those from the gold standard techniques. The assay can enable rapid and reagentless target detection and can be readily modified to measure other target analytes in vivo, such as glucose, ATP, uric acid, serotonin, insulin, cortisol, vanomycin, gentamicin, tyrosinamide, thrombin, micro-RNAs such as miR21 and miR210.

In one or more embodiments, the present disclosure provides a continuous and real-time biosensor that can provide insight into the health status of patients, and their response to therapeutics, in a non- or minimally invasive manner, which may enable health care systems to provide personalized and precision medicine. In one or more embodiments example, a Microneedle Aptamer-assisted Detector was developed and is described herein for minimally Invasive and Continuous Tracking, or MADICT, to address challenges in real-time biosensing. In one or more embodiments example, the Microneedle Detector may be a nucleic acid-assisted microneedle detector. In one or more embodiments, the MADICT may form the basis for a universal platform for multiplexed, rapid and in-line measurement of any target molecules in interstitial fluid (ISF). In one or more embodiments, the MADICT may comprise three main components: 1) Hydrogel microneedles (HMNs) for ISF extraction; 2) nucleic acid probes (e.g., aptamer probes, pDNA probes) for sensitive target detection; and 3) a semiconductor integrated chip (IC) and supporting electronics for miniaturized electrochemical measurement and wireless transmission of data.

Upon insertion into skin, the HMNs can rapidly swell once in contact with ISF, which facilitates a continuous diffusion of target molecules into the patch's needles, which are functionalized with the nucleic acid probes (e.g., aptamer probe, pDNA probe) that are a main component of the real-time biosensor. In one or more embodiments, the HMNs can also act as a working electrode (WE). In one or more embodiments, the HMNs can also act as a working electrode (WE) with the integration of a conductive material and a redox reporter coupled or linked to the probe, such as a nucleic acid probe (e.g., aptamer probes, pDNA probes). In one or more embodiments, incorporation of a conductive polymer, such as poly(3,4-ethylenedioxythiophene) polystyrene sulfonate (PEDOT:PSS), into the hydrogel network may improve the electrical properties of the HMNs, making the MNs according to one or more embodiments suitable for use as a working electrode in a biosensor.

The nucleic acid probes (e.g., aptamer probes, pDNA probes) can switch their conformation upon specific target binding. Non-specific binding of background chemicals do not appear to cause the nucleic acid probes (e.g., aptamer probes, pDNA probes) to switch its conformation, as the probe has only been observed responding to the binding of the target even when the background concentration is many orders of magnitude higher. This feature enables the presently described sensor to operate continuously in complex mixtures without sample preparation or added reagents.

The requirements for continuous tracking are challenging and the field faces specific problems. Some challenges presently faced include: the biosensor preferably does not use any batch processing, such as wash steps or addition of reagents; the detection mechanism should be generalizable to a wide range of targets; the sensing scheme should have adequate sensitivity, specificity and dynamic range in a short time-scale; biosensors preferably use simple instrumentation for point-of-care (POC) implementation; and finally, the biosensor should remain stable even after prolonged exposure to complex biological environments.

Continuous biosensing has revolutionized diabetes care through enabling continuous glucose measurement, however, the molecular based real-time biosensors are mainly limited to the enzymatic detection of a handful of biomolecules such as glucose, oxygen, and lactate. Another generation of biosensors can continuously measure other types of biomolecules in vivo, including a platform for continuous detection of small-molecule drugs in the bloodstream of live animals using an electrochemical sensor based on structure-switching aptamer probes. Further work on real-time biosensing involved real-time enzyme-linked immunosorbent assay which combines antibody and aptamer probes for continuous measurement of insulin and glucose in live animals. These platforms may demonstrate the feasibility of continuous in vivo molecular detection for a wide range of analytes, but they tend to have complicated instrumentation suitable only for a few hours monitoring and are invasive.

Microneedle-based transdermal devices are emerging to address the challenges of non/minimally invasive wearable biosensing, and can be potentially employed for point-of-care (POC) diagnosis and tracking. Microneedles (MN) enable ready access to dermal interstitial fluid (ISF), one of the more prevalent, accessible fluids in the body that contains important biomarkers for continuous monitoring. New advances have recently been made in exploiting hollow, metallic MN-based devices for real-time monitoring of various metabolites, electrolytes, and therapeutics, and toward the simultaneous multiplexed detection of key chemical markers. However, these sensors tend to be mainly limited to enzymatic based detection, which can hinder their performance for detection of analytes for which enzymes are not available. It has been found that hydrogel microneedles (HMNs), which have been mainly used for cosmetics and drug delivery applications, have potential for diagnostics where extracted ISF has been used for off-chip detection of different analytes. Indeed, HMN arrays are considered to possess several advantages, such as increased amount of ISF (10 μL vs 2 μL in hollow MN), lower fabrication cost and higher production yield when comparing to other MNs. However, they lacked in situ sensing.

In one or more embodiments of the present disclosure, the herein described MADICT combines HMN arrays with nucleic acid probes (e.g., aptamer probes, pDNA probes), which may address unmet challenges related to real-time biosensing. In one or more embodiments, the present disclosure may address the above-noted challenges for at least the following reasons. Nucleic acid probes (e.g., aptamer probes, pDNA probes) can enable continuous and reagentless measurement of any target molecule of interest. Nucleic acid probes (e.g., aptamer probes, pDNA probes) can be also selected for high sensitivity and specificity detection. Incorporating IC and supporting electronics for miniaturized electrochemical measurement and wireless transmission of data can enable monitoring in POC setting. In one or more embodiments, HMNs can act as a support matrix which can hinder the degradation of nucleic acid probes (e.g., aptamer probes, pDNA probes) even after exposure to ISF environments.

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein refers to the list that follows being non-exhaustive and may or may not include any other additional suitable items, for example one or more further feature(s), component(s) and/or ingredient(s) as appropriate.

The term “subject”, as used herein, refers to an animal, and can include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal. In a specific example, the subject is a human.

The term “linker” or “linker functional group” refers to a chemical moiety or chemical functional group that connects, or is used to connect two entities. For example, a phosphoramidite functional group may be used to connect a nucleic acid to a hydrogel; or, a PEG chain may connect a nucleic acid to a quencher. A skilled person will recognize how to select an appropriate linker, or linker functional group in view of the two entities to be connected, and the intended application/desired properties of the linked product.

Generally, the present disclosure provides microneedles for detecting a target. In another aspect, the present disclosure provides methods of producing microneedles. In another aspect, the present disclosure provides methods and apparatus using the microneedles herein disclosed. In other aspects, the present disclosure provides transdermal patches and methods of biosensing using the same.

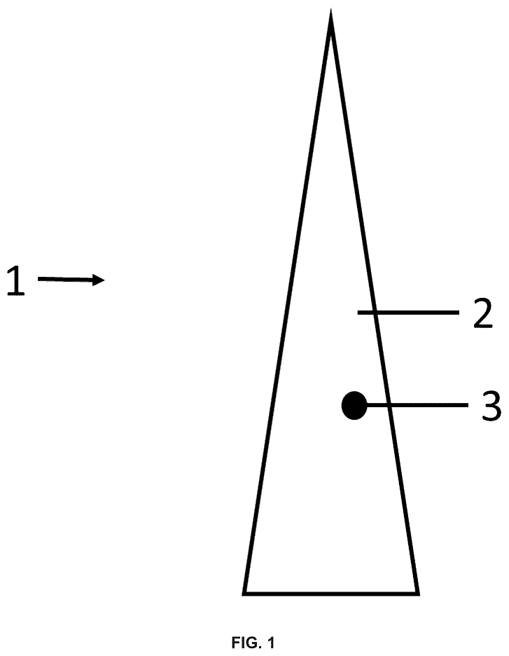

According to an embodiment, the microneedle of the present disclosure may comprise a hydrogel and a probe coupled to the hydrogel, the probe for generating a measurable signal in the presence of the target. The measurable signal may be generated in situ. In other words, the signal may be measurable without requiring other reagents (e.g., reagent-less) or processing steps. FIG. 1 shows an embodiment of a microneedle (1), comprising a hydrogel (2) and a probe (3).

Hydrogels

Microneedles described herein comprise a hydrogel. The hydrogel may be any suitable polymer or combinations thereof. The hydrogel preferably comprises functionalities that allow for crosslinking and/or functionalization, and it will be understood that any polymer with such functionalities may be used. The hydrogel may comprise a polymer comprising at least one C═C functionality, where the C═C functionality may allow for crosslinking and/or functionalization. The hydrogel may comprise an acrylated polymer, a methacrylated polymer, or a combination thereof. Suitable methacrylated polymers include but are not limited to methacrylated gelatin, methacrylated hyaluronic acid, methacrylated alginate, methacrylated chitosan, methacrylated collagen, methacrylated polyethylene glycol, methacrylated polyvinyl alcohol, methacrylated polylysine, and combinations thereof. The hydrogel may include a combination of polymers, and said polymers may have differing functionalities. The hydrogel may comprise functionalized hyaluronic acid, such as methacrylated hyaluronic acid. The hydrogel may comprise any suitable conductive material for communicating an electrochemical signal throughout the hydrogel, such as a conductive polymer or ionmer.

Probes

Microneedles described herein comprise a probe. The probe may be for generating a measurable signal in the presence of a target, alone or in communication with other components of the microneedle. The probe may be for generating a fluorescence signal or an electrochemical signal, in the presence of a target, alone or in communication with other components of the microneedle.

The probe may be coupled to the hydrogel by any suitable means or fashion, such as by bond, association, physisorption, intermolecular bonding, or complexation. The probe may be coupled to the hydrogel directly, or by a linker. If the probe is coupled to the hydrogel by a linker, the linker may be formed from a phosphoramidite functional group, such as an acrydite functional group. The probe may be covalently bonded to the hydrogel. The probe may be coupled to the hydrogel via hydrogen bonding, or other intermolecular interaction. The probe may be coupled to the hydrogel via physisorption, such as pi-pi stacking.

Any suitable probe may be used in the microneedles herein described. The probe may be any suitable moiety for generating a measurable signal. The probe preferably generates a measurable signal when bound to, associated with, or in proximity to the target. The probe may comprise a fluorophore, or be conjugated to a fluorophore. The probe may comprise a redox reporter, or be conjugated to a redox reporter.

The probe may comprise a nucleic acid. The nucleic acid may comprise an aptamer, single stranded complementary probe DNA, peptide nucleic acid, nucleic acid enzyme, or combinations thereof. The probe may comprise an aptamer, such as an aptamer that binds a target. The probe may comprise probe DNA (pDNA), wherein the pDNA is a pDNA that binds to the target. The pDNA may be suitable for any nucleic acid-based target, such as single stranded DNA or RNA to which the sequence of pDNA is complementary. The aptamer may comprise a linker functional group for coupling the probe to the hydrogel. The pDNA may comprise a linker functional group for coupling the probe to the hydrogel. The linker functional group may comprise a phosphoramidite functional group, such as an acrydite functional group. The aptamer may comprise, or be conjugated or linked to a fluorophore. The aptamer may comprise, or be conjugated or linked to a redox reporter. The pDNA may comprise, or be conjugated or linked to a fluorophore. The pDNA may comprise, or be conjugated or linked to a redox reporter. The aptamer may comprise any suitable sequence for binding a target of interest, such as a biomarker. Many such aptamers will be readily known by the person of skill in the art. The aptamer may comprise a sequence as listed in Table 1 or Table 2. It will be readily understood that, while a glucose aptamer, an ATP aptamer were tested herein, it is expected that aptamers for other targets can be used in their place. For example, insulin, cortisol, vancomycin, gentamicin aptamers could be used, such as those listed in Table 1. pDNA is a type of DNA probe that is single stranded, and targets single-stranded DNA or RNA. Generally, the pDNA sequence is complementary to its DNA or RNA target, such as micro-ribonucleic acids (miR). For example, see Tables 1 and 4. The pDNA may comprise any suitable sequence for binding a target of interest, such as a biomarker. Many such pDNA will be readily known by the person of skill in the art.

TABLE 1
Exemplary suitable aptamer sequences, and associated targets.

| Target | Sequence |
|---|---|
| Insulin | 5′GGTGGTGGGGGGGGTTGGTAGGGTGTCTTC3′ (SEQ ID NO. 1) |
| Cortisol | 5′AATGCGGGGTGGAGAATGGTTGCCGCA3′ (SEQ ID NO. 2) |
| Vancomycin | 5′CTCTCGGGACGACCGAGGGTACCGCAATAGTACTTATTGTTCGCCTATTGTGGGTCGGGTCGTCC |
| | C3′ (SEQ ID NO. 3) |
| Gentamicin | 5′GGGACTTGGTTTAGGTAATGAGTCCC3′ (SEQ ID NO. 4) |
| Glucose | ′5CTCTCGGGACGACCGTGTGTGTTGCTCTGTAACAGTGTCCATTGTCGTCCC3′ (SEQ ID NO. 5) |
| ATP | 5′CACCTGGGGGAGTATTGCGGAGGAAGG3′ (SEQ ID NO. 6) |
| L-tyrosinamide | 5′GGAGCTTGGATTGATGTGGTGTGTGAGTGCGGTGCCC3′ (SEQ ID NO. 7) |
| Thrombin | 5′GGTTGGTGTGGTTGG3′ (SEQ ID NO. 8) |
| Serotonin | 5′ GACTGGTAGGCAGATAGGGGAAGCTGATTCGATGCGTGGGTC3′ (SEQ ID NO. 9) |
| Uric acid | 5′CTCTCGACGACATTACGGGACCTTGCTAAAGGTGGAATTATGTCGT 3′ (SEQ ID NO. 10) |
| MIR210 pDNA | /5Cy3/TCAGCCGCTGTCACACGCACAG/3AmMO/ (SEQ ID NO. 11) |
| MIR21 pDNA | /5Cy3/TCAACATCAGTCTGATAAGCTA/3AmMO/ (SEQ ID NO. 12) |

Cy3-Cyanine 3 modification
5AmMC6 is 5′ Amino Modifier C6 modification
3AmMO is 3′ Amino Modifier modification

The probe may further comprise a quencher. The probe may be reversibly bound to a quencher. The probe may be tethered to the quencher via a linker at one end, and may be reversibly bound to the quencher at another end. If the probe comprises a fluorophore, for example, the quencher may be a quencher of the fluorophore. If the probe is an aptamer, the quencher may comprise a sequence complimentary to at least a portion of the aptamer sequence. If the probe is pDNA, the quencher may comprise a sequence complimentary to at least a portion of the pDNA sequence. The quencher may be any suitable quencher for reducing the signal of the probe in the absence of the target and/or enhancing the measurable signal of the probe in the presence of the target. The quencher may comprise a moiety that binds competitively to the probe, and preferably the target binds the probe more competitively, favourably, or strongly as compared to the quencher. The quencher may comprise a competitor strand of the aptamer, or the pDNA. The quencher may comprise a sequence as listed in Tables 1 to 3. The quencher may comprise a sequence partially or completely complimentary to at least a portion of a sequence listed in Tables 1 to 3, for example, if the sequence listed is an aptamer or pDNA. The quencher may comprise a moiety that conjugates competitively with the probe, and preferably the target binds the probe more competitively, favourably, or strongly as compared to the quencher.

The probe may be tethered to a quencher at one end, and may be reversibly bound to the quencher at another end. The probe may be tethered to the quencher via covalent bonding, intermolecular bonding, physical adsorption (such as pi-pi stacking, hydrophobic interactions), or conjugation. The quencher may comprise a graphene-based material, such as graphene-oxide (GO) nanosheets, graphene-oxide (GO) nanoparticles, graphene-oxide (GO) nanocomposites, or a combination thereof. The quencher may comprise a graphene-based material, such as graphene-oxide (GO) nanosheets, graphene-oxide (GO) nanoparticles, graphene-oxide (GO) nanocomposites, or a combination thereof, tethered to the probe via covalent bond, physical adsorption, or conjugation. If the probe is tethered to a quencher, the quencher may conjugate competitively with the probe, and the probe may disassociate from, or become distanced from the quencher in the presence of a target that preferably binds the probe more competitively, favourably, or strongly as compared to the quencher.

Nevertheless, it will be understood that any suitable quencher, such as a quencher complimentary to any of the above-noted aptamer or pDNA probes, or graphene-materials (e.g., graphene-oxide (GO) nanosheets, graphene-oxide (GO) nanoparticles, graphene-oxide (GO) nanocomposites, or a combination thereof) conjugated to probes, is within the scope of the present disclosure.

The probe may comprise, or may be coupled to an electroactive species, a redox active species, or a combination thereof. The probe may comprised, or be coupled a redox reporter. The redox reporter may comprise aromatic species. The probe may comprise, or may be coupled to metal nanoparticles. In one or more embodiments, when the probe comprises a nucleic acid, such as an nucleic acid that binds a target, the nucleic acid may comprise, or may be coupled to an electroactive species, a redox active species, a redox reporter, metal nanoparticles, or a combination thereof. In one or more embodiments, when the probe comprises an aptamer, such as an aptamer that binds a target, the aptamer may comprise, or may be coupled to an electroactive species, a redox active species, a redox reporter, metal nanoparticles, or a combination thereof. In one or more embodiments, when the probe comprises pDNA, such as pDNA that binds a target, the pDNA may comprise, or may be coupled to an electroactive species, a redox active species, a redox reporter, metal nanoparticles, or a combination thereof. In one or more embodiments, the electroactive species, redox active species, redox reporter, metal nanoparticles, or a combination thereof, may communicate, or assist with communicating, an electrochemical signal through the hydrogel. Many such electroactive species, redox active species, redox reporters, or metal nanoparticles will be readily known by the person of skill in the art. In an example, metal nanoparticles may comprise platinum, silver, gold, palladium, or combinations thereof. The metal nanoparticles may comprise platinum and/or silver. In another example, the redox reporter may comprise an aromatic species. The redox reporter may comprise methylene blue, ferrocene, or a combination thereof.

In embodiments where the probe may comprise, or may be coupled to an electroactive species, a redox active species, or a combination thereof, such as a redox reporter, the probe may not comprise a quencher. In embodiments where the probe may comprise, or may be coupled to an electroactive species, a redox active species, or a combination thereof, such as a redox reporter, the probe may comprise a quencher, where the quencher acts as support for the probe or as a conductive material and may not quench the measurable signal; for example, wherein the quencher comprises graphene-oxide (GO) nanosheets, graphene-oxide (GO) nanoparticles, graphene-oxide (GO) nanocomposites, or a combination thereof. Where the probe may comprise, or may be coupled to an electroactive species, a redox active species, or a combination thereof, the probe may bind with a target and undergo a conformational change. In undergoing this conformational change, the electroactive species, redox active species, or combination thereof of the probe may be brought closer to conductive materials in the microneedle. The conductive materials may comprise metal nanoparticles, graphene-based materials, conductive polymers, or ionomers, where the conductive polymer or ionomer may comprise poly(3,4-ethylenedioxythiophene) polystyrene sulfonate; polyacetylene; polypyrrole; polyindole; polyaniline; a copolymer thereof; or a combination thereof; and the graphene-based material may comprise ferrocene, graphene-oxide (GO) nanosheets, graphene-oxide (GO) nanoparticles, graphene-oxide (GO) nanocomposites, or a combination thereof. Bringing the electroactive species, redox active species, or combination thereof of the probe closer to conductive materials may generate and/or transmit an electrical signal for detection.

Other Components

The microneedles herein described may comprise other components. For example, the microneedle or the hydrogel may comprise a nanoparticle. In one or more embodiments, the probe may be coupled to the nanoparticle, and the nanoparticle coupled or associated with the hydrogel.

In one or more embodiments, the nanoparticle may be a conductive material that's integrated into the hydrogel, or a component thereof. In one or more embodiments, the nanoparticle may communicate, or assist with communicating, an electrochemical signal through the hydrogel. In one or more embodiments, the nanoparticle may enhance electrical properties of the microneedle. The nanoparticle may comprise any suitable metal, such as platinum, silver, gold, palladium, or combinations thereof. The metal nanoparticles may be platinum nanoparticles or platinum and silver nanoparticles. Graphene may be used in addition to, or in place of, metal nanoparticles in the microneedle. The microneedle may comprise any other suitable components.

Measurable Signal

The microneedles herein described are for generating a measurable signal in the presence of a target, such as a biomolecule or a biomarker. The measurable signal may be fluorescence. The measurable signal may be an electrochemical signal. The electrochemical signal may be generated in-situ by the probe. The electrochemical signal may be communicated through the hydrogel, such as with the assistance of a conductive material.

It will be understood that other measurable signals may be used without departing from the spirit of the present disclosure. For example, the measurable signal may be an electrochemical signal extracting from a redox reporter coupled to the probe. The redox reporter may be methylene blue, ferrocene, or combination thereof.

Targets

The microneedles and transdermal patches herein described may be used to detect one or more targets. The target may be any suitable molecule for detection. The target may be a biomolecule or biomarker, such as a biomolecule present in interstitial fluid. The target may comprise small biomolecules, proteins, or micro ribonucleic acids. The target may comprises glucose, ATP, uric acid, serotonin, insulin, cortisol, vanomycin, gentamicin, tyrosinamide, thrombin, micro-RNAs such as miR21 and miR210. The target may comprise small biomolecules uric acid (UA), serotonin, or glucose. The target may comprise protein insulin. The target may comprise micro ribonucleic acids miR21, or miR210. The target may be adenosine triphosphate or glucose. It will be understood that while aptamer or pDNA probes for certain targets were exemplified herein, the same principle may be used to detect other targets, such as any molecule with a known aptamer or pDNA. It will be understood that any molecule or ion suitable for generating an electrochemical signal in the presence of the probe may be detected, such as a molecule or ion that undergoes a redox reaction with the probe. Further, in embodiments where the probe may be coupled to an electroactive species, a redox active species, a redox reporter, metal nanoparticles, or a combination thereof, it will be understood that any target, such as an ion, or a molecule with a known aptamer, suitable for generating an electrochemical signal in the presence of the probe may be detected.

Conductive Materials

The microneedles herein described may comprise a conductive material. The conductive material may be any suitable material for communicating a signal from the probe through the hydrogel. For example, the conductive material may communicate an electrochemical signal of the probe through the hydrogel and to an electrical wire associated with the microneedle. The conductive material may be a nanoparticle, such as a metal nanoparticle, or graphene. The nanoparticle or graphene may be embedded within the hydrogel's 3D network. The conductive material may be a conductive polymer, such as an ionomer. Any suitable conductive polymer may be used, such as poly(3,4-ethylenedioxythiophene) polystyrene sulfonate, polyacetylene, polypyrrole, polyindole and polyaniline and their copolymers. The conductive material or polymer may be mixed with, embedded in, conjugated with or covalently linked (e.g. by crosslinking) to the hydrogel.

The conductive material may comprise metal nanoparticles, graphene-based materials, conductive polymers, or ionomers. In an embodiment, the conductive polymer or ionomer comprises poly(3,4-ethylenedioxythiophene) polystyrene sulfonate; polyacetylene; polypyrrole; polyindole; polyaniline; a copolymer thereof; or a combination thereof. In an embodiment, the graphene-based material comprises ferrocene, graphene-oxide (GO) nanosheets, graphene-oxide (GO) nanoparticles, graphene-oxide (GO) nanocomposites, or a combination thereof.

Methods of producing Microneedles

In one or more aspects, there is provided a method of producing a microneedle. The method may comprise combining a functionalized hydrogel, a probe precursor, optionally a conductive material, and a crosslinking agent in a mold; and exposing the mixture in the mold to UV light to link at least a portion of the probe to the functionalized hydrogel and to form a crosslinked material. The method optionally comprises functionalizing a hydrogel to form the functionalized hydrogel, such as reacting hyaluronic acid with methacrylic anhydride to form methacrylated hyaluronic acid. The method may further comprise washing and/or purification steps. The method may comprise washing the microneedle to remove excess or unbound probe or other reagent. The method may comprise removing the crosslinked material from the mold, and/or further exposing the unmolded crosslinked material to UV light. The method optionally comprises washing the crosslinked material to remove unbound probe. The crosslinking agent may be any suitable reagent, such as N,N′-methylenebisacrylamide. The method may comprise combining functionalized hydrogel, probe precursor, and crosslinking agent with a photoinitiator in the mold. The crosslinking may occur in the presence of a photoinitiator. The photoinitiator may catalyze the crosslinking. The hydrogel may be cast in any suitable way to form a microneedle, such as by mold. The mold may be a negative polydimethylsiloxane mold. In one embodiment, combining the functionalized hydrogel, the probe precursor, and the crosslinking agent in the mold comprises:
dissolving about 50:1 to about 10:1 (wt/wt) of functionalized hydrogel:crosslinking agent in buffer to form a functionalized hydrogel solution;
optionally adding a conductive material to the functionalized hydrogel solution;
optionally degassing the functionalized hydrogel solution;
adding the functionalized hydrogel solution to the mold;
partially drying the functionalized hydrogel solution in the mold;
optionally, adding further functionalized hydrogel solution to the mold;
adding the probe precursor to the mold; and
optionally, drying the mixture in the mold further.

It will be understood that any suitable conditions may be used. The ratio of functionalized hydrogel:crosslinking agent may be about 50:1 to about 10:1, such as about 10:1, or about 20:1, or about 30:1, or about 40:1 or about 50:1. The probe precursor solution may comprise a nucleic acid. The probe precursor solution may comprise a nucleic acid and a quencher. The probe precursor solution may comprise a nucleic acid that comprises, or is coupled to an electroactive species, a redox active species, or a combination thereof, such as a redox reporter. The probe precursor solution may comprise a nucleic acid and quencher (e.g., an aptamer and a quencher, or pDNA and a quencher), for example, in a ratio of about 1:5 to about 1:20, such as about 1:5 or about 1:10 or about 1:15 or about 1:20. Degassing the functionalized hydrogel solution may be done at any suitable stage of the method or process, and degassing may comprise centrifuge, sonication and/or vacuum. Drying the functionalized hydrogel solution in the mold may occur for any suitable amount of time, and in one or more stages. For example, to the partially-dried functionalized hydrogel solution in the mold may be added additional functionalized hydrogel solution for further drying. This process may be repeated one or more times. The functionalized hydrogel solution may be exposed to any suitable conditions for curing or crosslinking, such as UV light. The mixture in the mold may be exposed to UV light, such as at a wavelength of about 360 nm for a suitable amount of time, such as about 15 minutes. For example, the mixture may be exposed to light of 200 nm to 400 nm, such as about 360 nm. For example, the mixture may be exposed to curing conditions, such as UV light, for about 1 minute to about 1 hour, such as about 10-20 minutes.

The method may comprise combining a functionalized hydrogel, a probe precursor, and a conductive material to form a mixture and forming said mixture into the microneedle. The method may comprise curing the mixture, such as by crosslinking the mixture in a mold to form a crosslinked material. The crosslinking may be induced by any suitable means, such as by exposing the mixture to UV light. In one embodiment, the method comprises combining a functionalized hydrogel, a probe precursor, and a conductive material to form a mixture; and crosslinking the mixture in a mold to form a crosslinked material. Crosslinking may involve exposing the mixture to UV light. The method may comprise one or more drying steps, such as drying the mixture in the mold before or after crosslinking. Combining the functionalized hydrogel, probe precursor, and the conductive material may be done by any suitable means or procedure. The method may further include degassing, such as degassing the mixture in the mold by any suitable means, such as by centrifuge, sonication, and or applying vacuum. The method may further comprise combining the functionalized hydrogel, the probe precursor, and the conductive material with a metal nanoparticle or metal nanoparticle precursor. The metal nanoparticle precursor may be a metal salt, such as a Pt, Pd, Au, or Ag salt. The metal nanoparticle may be a metal nitrate or metal chloride, for example, silver nitrate and/or platinum sodium chloride.

Properties of Microneedles

In one or more aspects, there is provided a microneedle obtainable or obtained by the methods herein disclosed. The microneedle may have a length of about 300 μm to about 1000 μm, such as about 800 μm. The microneedle may be produced as a plurality of microneedles. The plurality of microneedles may be in any suitable arrangement, such as a grid. The plurality of microneedles may be connected, for example, the microneedles may be cast by a mold that includes a base layer from which the microneedles extend. The plurality of microneedles may be in a grid of about 10 to about 500. The plurality of microneedles may be in a square grid, such as a grid of 3×3, or 4×4, or 5×5, etc. The plurality of microneedles may be in a rectangular grid, such as a grid of 3×4, or 3×5, or 3×6, etc. or 4×5, or 4×6, etc. The plurality of microneedles may be in an irregularly shaped grid, or any suitable shape or pattern.

The plurality of microneedles may be individually spaced apart, such as at a distance of about 100 μm to about 1000 μm, such as about 500 μm.

Apparatus

In one or more aspects, there is provided an apparatus for detecting a target in a sample. The apparatus may comprise a microneedle as disclosed herein together with a detector for detecting the measurable signal. The detector may be any suitable detector, such as a fluorimeter for detecting a fluorescent signal, etc. The detector may be a smartphone-based detector for detecting a fluorescent signal, etc. The smartphone detector may be a miniaturized optic and imager system that includes: a laser diode for exciting the fluorophore comprised by, or conjugated to a probe of a HMN as described herein or transdermal patch comprising the same, a microscopic objective for magnification, a filter such as a bandpass filter for passing frequencies within a certain range and rejecting (attenuating) frequencies outside that range, and a lens such as a camera lens for focusing emitted light. Any emitted fluorescent light from the HMN or patch can be magnified by the objective, passed through the filter to filter out background excitations, collected by the camera lens, and visualized by the smartphone. The apparatus may comprise a microneedle as disclosed herein together with a detector for detecting the electrochemical signal. The detector may be any suitable detector, such as a potentiostat.

The apparatus may further comprise a reference electrode and/or a counter electrode, or they may be provided separately.

The sample may be any suitable sample, such as a solution comprising the target, a biological sample or solution thereof. The sample may have been taken from a subject, or the apparatus may be used with a transdermal patch for biosensing, wherein the sample is the subject's interstitial fluid.

Transdermal Patch

In one or more aspects, there is provided a transdermal patch comprising a microneedle according to any one of more embodiments of the present disclosure. The transdermal patch may comprise other components, such as adhesive to attach the patch and/or a bandage.

In the transdermal patch, the microneedle as described herein may act as a working electrode. The transdermal patch may thus further comprise a reference electrode and/or a counter electrode, or these electrodes may be provided separately, such as on a separate patch. The reference electrode and/or the counter electrode may also be microneedle electrodes, or they may be any suitable electrode for use in the system. The reference electrode may be a microneedle electrode comprising Ag/AgCl. The counter electrode may be a microneedle electrode comprising Au. The transdermal patch may comprise three electrodes (a working electrode, counter electrode, and reference electrode) or a plurality of each. The transdermal patch may comprise other components, such as adhesive to attach the patch and/or a bandage. The electrodes may be localized on one area of the patch, such as in a side-by-side arrangement. Each electrode may have its own associated wiring.

Biosensing

In one or more aspects, there is provided a method for transdermal biosensing, or a use of the microneedles herein disclosed for transdermal biosensing. The method may comprise applying a transdermal patch or any suitable means of applying microneedles as herein described. The method may comprise applying the transdermal patch to any suitable location of a subject, such as arm, leg, abdomen, etc. The method or use for biosensing may include detecting the measurable signal and associating the measurable signal to the concentration of the target in the subject. Alternatively, the method or use may be for simply identifying the presence of a given level of the target in the subject, as opposed to the exact concentration. Detecting the measurable signal may be done completely in situ; in other words, the detection may be done without further processing steps. Detecting may be reagentless. Detecting may occur without requiring removal of the transdermal patch, or without further processing of the transdermal patch. Detecting may occur while the transdermal patch is applied, or in contact with the subject. Detecting the measurable signal may comprise measuring the fluorescence intensity of the probe, measuring voltammetry or amperometry, or any other suitable measurement of the microneedle system. Associating the measurable signal may comprise comparing a measured intensity of the measurable signal to a calibration curve of measured intensities of known concentrations of the target. It will be understood that any suitable means of associating the measurable signal to the presence or absence (or concentration) of the target may be used.

Herein, there is described:
1. A microneedle for detecting a target, the microneedle comprising: a hydrogel; a probe coupled to the hydrogel, the probe for generating a measurable signal in the presence of the target.
2. The microneedle of item 1, wherein the measurable signal is generated in situ.
3. The microneedle of item 1 or 2, wherein the hydrogel comprises a polymer comprising at least one C═C functionality.
4. The microneedle of any one of items 1 to 3, wherein the hydrogel comprises an acrylated polymer, a methacrylated polymer, or a combination thereof.
5. The microneedle of item 4, wherein the hydrogel comprises methacrylated gelatin, methacrylated hyaluronic acid, methacrylated alginate, methacrylated chitosan, methacrylated collagen, or a combination thereof.
6. The microneedle of any one of items 1 to 5, wherein the hydrogel comprises functionalized hyaluronic acid.
7. The microneedle of any one of items 1 to 6, wherein the hydrogel comprises methacrylated hyaluronic acid.
8. The microneedle of any one of items 1 to 7, wherein the probe coupled to the hydrogel comprises the probe being coupled to the hydrogel by a linker.
9. The microneedle of item 8, wherein the linker is formed from a phosphoramidite functional group, such as an acrydite functional group.
10. The microneedle of any one of items 1 to 9, wherein the probe coupled to the hydrogel comprises the probe being covalently bonded to the hydrogel.
11. The microneedle of any one of items 1 to 10, wherein the hydrogel further comprises a nanoparticle.
12. The microneedle of item 11, wherein the probe coupled to the hydrogel comprises the probe being coupled to the nanoparticle.
13. The microneedle of any one of items 1 to 12, wherein the probe comprises an aptamer.
14. The microneedle of item 13, wherein the aptamer is an aptamer that binds to the target.
15. The microneedle of item 13 or 14, wherein the aptamer comprises a sequence according to a SEQ ID No. herein disclosed.
16. The microneedle of any one of items 13 to 15, wherein the aptamer comprises a linker functional group for coupling the probe to the hydrogel.
17. The microneedle of item 16, wherein the linker functional group comprises a phosphoramidite functional group.
18. The microneedle of item 16 or 17, wherein the linker functional group comprises an acrydite functional group.
19. The microneedle of any one of items 1 to 18, wherein the probe comprises a fluorophore.
20. The microneedle of any one of items 1 to 19, wherein the probe comprises an aptamer, and the aptamer comprises a fluorophore or is linked to a fluorophore.
21. The microneedle of any one of items 1 to 20, wherein the probe is reversibly bound to a quencher.
22. The microneedle of item 21, wherein the probe is an aptamer and the quencher comprises a sequence partially or fully complimentary to at least a portion of the aptamer sequence.
23. The microneedle of item 21 or 22, wherein the quencher comprises a sequence according to a SEQ ID No. herein disclosed.
24. The microneedle of any one of items 1 to 23, wherein the measurable signal is fluorescence.
25. The microneedle of any one of items 1 to 24, wherein the target is a biomolecule present in interstitial fluid.
26. The microneedle of any one of items 1 to 25, wherein the target is adenosine triphosphate.
27. The microneedle of any one of items 1 to 26, wherein the target is glucose.
28. The microneedle of any one of items 1 to 27, wherein the microneedle has a length of about 300 μm to about 1000 μm, such as about 800 μm.
29. A method of producing a microneedle, the method comprising: combining a functionalized hydrogel, a probe precursor, and a crosslinking agent in a mold; and exposing the mixture in the mold to UV light to link at least a portion of the probe to the functionalized hydrogel and to form a crosslinked material.
30. The method of item 29, further comprising functionalizing a hydrogel to form the functionalized hydrogel.
31. The method of item 29 or 30, further comprising: removing the crosslinked material from the mold; and further exposing the unmolded crosslinked material to UV light.
32. The method of any one of items 29 to 31, further comprising washing the crosslinked material to remove unbound probes.
33. The method of any one of items 29 to 32, wherein combining the functionalized hydrogel, the probe precursor, and the crosslinking agent in the mold comprises: dissolving about 50:1 to about 10:1 (wt/wt) of functionalized hydrogel:crosslinking agent in a buffer to form a functionalized hydrogel solution; optionally degassing the functionalized hydrogel solution; adding the functionalized hydrogel solution to the mold; partially drying the functionalized hydrogel solution in the mold; optionally, adding further functionalized hydrogel solution to the mold; adding the probe precursor to the mold; and optionally, drying the mixture in the mold further.
34. The method of any one of items 29 to 33, wherein the probe precursor is a solution of aptamer and quencher, preferably in a ratio of about 1:5 to about 1:20, such as a 1:10 ratio.
35. The method of any one of items 29 to 34, wherein exposing the mixture in the mold to UV light comprises exposing the mixture to light of about 200 nm to about 400 nm, preferably about 360 nm light; and preferably for about 1 min to about 1 hour, such as about 10 to about 20 min.
36. The method of any one of items 29 to 35, wherein the hydrogel comprises hyaluronic acid.
37. The method of item 36, wherein functionalizing the hydrogel comprises reacting hyaluronic acid with methacrylic anhydride to form methacrylated hyaluronic acid.
38. The method of any one of items 29 to 37, wherein combining the functionalized hydrogel, the probe precursor and the crosslinking agent in the mold further comprises adding a photoinitiator to the mixture in the mold.
39. The method of any one of items 29 to 38, wherein the crosslinking agent comprises N,N′-methylenebisacrylamide.
40. The method of any one of items 29 to 39, wherein the mold is a negative polydimethylsiloxane mold.
41. A microneedle obtainable or obtained by the method according to any one of items 29 to 40.
42. An apparatus for detecting a target in a sample, the apparatus comprising: the microneedle according to any one of items 1 to 28 or 41; and a detector for detecting the measurable signal.
43. The apparatus of item 42, wherein the detector is a fluorimeter.
44. A transdermal patch comprising the microneedle according to any one of items 1 to 28 or 41.
45. A method for transdermal biosensing of a target in a subject, the method comprising: applying the transdermal patch according to item 44; detecting the measurable signal; and associating the measurable signal to the concentration of the target in the subject.
46. The method of item 45, wherein detecting the measurable signal is in situ.
47. The method of item 45 or 46, wherein detecting the measurable signal is reagentless.
48. The method of any one of items 45 to 47, wherein detecting the measurable signal occurs without requiring removal of the transdermal patch.
49. The method of any one of items 45 to 48, wherein detecting the measurable signal occurs while the transdermal patch is applied to the subject.
50. The method of any one of items 45 to 49, wherein detecting the measurable signal occurs in the absence of further processing of the transdermal patch.
51. The method of any one of items 45 to 50, wherein detecting the measurable signal comprises measuring the fluorescence intensity of the probe.
52. The method of any one of items 45 to 51, wherein associating the measurable signal comprises comparing a measured intensity of the measurable signal to a calibration curve of measured intensities of known concentrations of the target.

Herein, there is also described:
1. A microneedle for detecting a target, the microneedle comprising: a hydrogel; a probe coupled to the hydrogel, the probe for generating a measurable signal in the presence of the target.
2. The microneedle of item 1, wherein the hydrogel comprises a polymer comprising at least one C═C functionality.
3. The microneedle of item 1 or 2, wherein the hydrogel comprises a polymer comprising an acrylated polymer, a methacrylated polymer, or a combination thereof.
4. The microneedle of any one of items 1 to 3, wherein the hydrogel comprises a polymer comprising methacrylated gelatin, methacrylated hyaluronic acid, methacrylated alginate, methacrylated chitosan, methacrylated collagen, methacrylated polyethylene glycol, methacrylated polyvinyl alcohol, methacrylated polylysine, or a combination thereof.
5. The microneedle of any one of items 1 to 4, wherein the hydrogel further comprises a conductive polymer, an ionomer, or a combination thereof.
6. The microneedle of any one of items 1 to 5, wherein the conductive polymer, an ionomer, or a combination thereof comprises poly(3,4-ethylenedioxythiophene) polystyrene sulfonate; polyacetylene; polypyrrole; polyindole; polyaniline; a copolymer thereof; or a combination thereof.
7. The microneedle any one of items 1 to 6, wherein the probe coupled to the hydrogel comprises the probe being coupled to the hydrogel by covalent bonding, intermolecular bonding, physisorption, complexation, a linker; or a combination thereof.
8. The microneedle of any one of items 1 to 7, wherein the probe comprises a nucleic acid, wherein the nucleic acid is an nucleic acid that binds to the target.
9. The microneedle of item 8, wherein the nucleic acid comprises an aptamer, single stranded complementary probe DNA, peptide nucleic acid, nucleic acid enzyme, or combinations thereof.
10. The microneedle of item 8 or 9, wherein the nucleic acid comprises a linker functional group for coupling the probe to the hydrogel.
11. The microneedle of item 10, wherein the linker functional group comprises a phosphoramidite functional group; or an acrydite functional group.
12. The microneedle of any one of items 1 to 11, wherein the probe comprises a fluorophore.
13. The microneedle of item 12, wherein the probe comprises a nucleic acid, and the nucleic acid comprises a fluorophore or is linked to a fluorophore.
14. The microneedle of any one of items 1 to 13, wherein the probe further comprises a quencher.
15. The microneedle of item 14, wherein the probe is reversibly bound to a quencher.
16. The microneedle of item 14 or 15, wherein the probe is tethered to the quencher via covalent bonding, intermolecular bonding, physical adsorption, conjugation, or a combination thereof.
17. The microneedle of any one of items 14 to 16, wherein the probe comprises a nucleic acid and the quencher comprises a sequence partially or fully complimentary to at least a portion of the nucleic acid sequence.
18. The microneedle of any one of items 14 to 16, wherein the probe comprises a nucleic acid and the quencher comprises a graphene-based material.
19. The microneedle of item 18, wherein the graphene-based material comprises graphene-oxide (GO) nanosheets, graphene-oxide (GO) nanoparticles, graphene-oxide (GO) nanocomposites, or a combination thereof.
20. The microneedle of any one of items 12 to 19, wherein the measurable signal is fluorescence.
21. The microneedle of any one of items 1 to 11, wherein the probe comprises an electroactive species, a redox active species, or a combination thereof, such as a redox reporter.
22. The microneedle of item 21, wherein the probe comprises a nucleic acid, and the nucleic acid comprises an electroactive species, a redox active species, or a combination thereof; or is linked to an electroactive species, a redox active species, or a combination thereof.
23. The microneedle of item 21 or 22, wherein the measurable signal is an electrochemical signal.
24. The microneedle of any one of items 1 to 23, wherein the target comprises a biomolecule present in interstitial fluid.
25. The microneedle of item 24, wherein the target comprises small biomolecules, proteins, or micro ribonucleic acids.
26. The microneedle of item 24 or 25, wherein cortisol, vanomycin, gentamicin, tyrosinamide, thrombin, micro-RNA miR21, micro-RNA miR210, uric acid (UA), serotonin, insulin, adenosine triphosphate, or glucose.
27. The microneedle of any one of items 1 to 26, wherein the microneedle has a length of about 300 μm to about 1000 μm, such as about 800 μm.
28. The microneedle of any one of items 1 to 27, further comprising a conductive material.
29. The microneedle of item 28, wherein the conductive material comprises a metal nanoparticle, graphene-based material, conductive polymer, or an ionomer; or a combination thereof.
30. The microneedle of item 29, wherein the graphene-based material comprises ferrocene, graphene-oxide (GO) nanosheets, graphene-oxide (GO) nanoparticles, graphene-oxide (GO) nanocomposites, or a combination thereof.
31. The microneedle of item 29 or 30, wherein the conductive polymer, ionomer, or combination thereof comprises poly(3,4-ethylenedioxythiophene) polystyrene sulfonate; polyacetylene; polypyrrole; polyindole; polyaniline; a copolymer thereof; or a combination thereof.
32. A method of producing a microneedle, the method comprising: combining a functionalized hydrogel, a probe precursor, optionally a conductive material, and a crosslinking agent in a mold; and exposing the mixture in the mold to UV light to link at least a portion of the probe to the functionalized hydrogel and to form a crosslinked material.
33. The method of item 32, further comprising: removing the crosslinked material from the mold; and further exposing the unmolded crosslinked material to UV light.
34. The method of item 32 or 33, wherein combining the functionalized hydrogel, the probe precursor, optionally a conductive material, and the crosslinking agent in the mold comprises: dissolving about 50:1 to about 10:1 (wt/wt) of functionalized hydrogel:crosslinking agent in a buffer to form a functionalized hydrogel solution; optionally adding a conductive material to the functionalized hydrogel solution; optionally degassing the functionalized hydrogel solution; adding the functionalized hydrogel solution to the mold; partially drying the functionalized hydrogel solution in the mold; optionally, adding further functionalized hydrogel solution to the mold; adding the probe precursor to the mold; and optionally, drying the mixture in the mold further.
35. The method of any one of items 32 to 34, wherein the probe precursor comprises a solution of nucleic acid and optionally a quencher.
36. The method of any one of items 32 to 35, wherein exposing the mixture in the mold to UV light comprises exposing the mixture to light of about 200 nm to about 400 nm, preferably about 360 nm light; and preferably for about 1 min to about 1 hour, such as about 10 to about 20 min.
37. The method of any one of items 32 to 36, wherein the hydrogel comprises hyaluronic acid; and functionalizing functionalizing the hydrogel comprises reacting hyaluronic acid with methacrylic anhydride to form methacrylated hyaluronic acid.
38. The method of any one of items 32 to 36, wherein the optional conductive material comprises metal nanoparticles, graphene-based material, or a conductive polymer or ionomer, such as poly(3,4-ethylenedioxythiophene) polystyrene sulfonate, polyacetylene, polypyrrole, polyindole, polyaniline, or copolymers thereof.
39. The method of any one of items 32 to 38, wherein the mold is a negative polydimethylsiloxane mold.
40. An apparatus for detecting a target in a sample, the apparatus comprising: the microneedle according to any one of items 1 to 31; and a detector for detecting the measurable signal.
41. A transdermal patch comprising the microneedle according to any one of items 1 to 31.
42. A method for transdermal biosensing of a target in a subject, the method comprising: applying the transdermal patch according to item 41; detecting the measurable signal; and associating the measurable signal to the concentration of the target in the subject.
43. The method of item 42, wherein detecting the measurable signal is reagentless.
44. The method of item 42 or 43, wherein detecting the measurable signal comprises measuring the fluorescence intensity of the probe; or measuring an electrochemical signal. 45. The method of item 44, wherein associating the measurable signal comprises comparing a measured intensity of the measurable signal to a calibration curve of measured intensities of known concentrations of the target.

Herein, there is also described:
1. A microneedle for detecting a target, the microneedle comprising: a hydrogel; a probe coupled to the hydrogel, the probe for generating a measurable signal in the presence of the target.
2. The microneedle of item 1, wherein the hydrogel comprises a polymer comprising at least one C═C functionality; an acrylated polymer, a methacrylated polymer, or a combination thereof; and/or methacrylated gelatin, methacrylated hyaluronic acid, methacrylated alginate, methacrylated chitosan, methacrylated collagen, methacrylated polyethylene glycol, methacrylated polyvinyl alcohol, methacrylated polylysine, or a combination thereof.
3. The microneedle of item 1 or 2, wherein the hydrogel further comprises a conductive polymer, an ionomer, or a combination thereof.
4. The microneedle of any one of items 1 to 3, wherein the conductive polymer, an ionomer, or a combination thereof comprises poly(3,4-ethylenedioxythiophene) polystyrene sulfonate; polyacetylene; polypyrrole; polyindole; polyaniline; a copolymer thereof; or a combination thereof.
5. The microneedle any one of items 1 to 4, wherein the probe coupled to the hydrogel comprises the probe being coupled to the hydrogel by intermolecular bonding, physisorption, complexation, or a combination thereof.
6. The microneedle of any one of items 1 to 5, wherein the probe comprises a nucleic acid, wherein the nucleic acid is an nucleic acid that binds to the target.
7. The microneedle of item 6, wherein the nucleic acid comprises an aptamer, single stranded complementary probe DNA, peptide nucleic acid, nucleic acid enzyme, or combinations thereof.
8. The microneedle of any one of items 1 to 7, wherein the probe comprises a fluorophore.
9. The microneedle of item 8, wherein the probe comprises a nucleic acid, and the nucleic acid comprises a fluorophore or is linked to a fluorophore.
10. The microneedle of any one of items 1 to 9, wherein the probe further comprises a quencher.
11. The microneedle of item 10, wherein the probe is tethered to the quencher via covalent bonding, intermolecular bonding, physical adsorption, conjugation, or a combination thereof.
12. The microneedle of any one of items 1 to 11, wherein the probe comprises a nucleic acid and the quencher comprises a graphene-based material.
13. The microneedle of item 12, wherein the graphene-based material comprises graphene-oxide (GO) nanosheets, graphene-oxide (GO) nanoparticles, graphene-oxide (GO) nanocomposites, or a combination thereof.
14. The microneedle of any one of items 1 to 13, wherein the measurable signal is fluorescence.
15. The microneedle of any one of items 1 to 13, wherein the probe comprises an electroactive species, a redox active species, or a combination thereof, such as a redox reporter.
16. The microneedle of item 15, wherein the probe comprises a nucleic acid, and the nucleic acid comprises an electroactive species, a redox active species, or a combination thereof or is linked to an electroactive species, a redox active species, or a combination thereof.
17. The microneedle of item 15 or 16, wherein the measurable signal is an electrochemical signal.
18. The microneedle of any one of items 1 to 17, wherein the target comprises a biomolecule present in interstitial fluid.
19. The microneedle of item 18, wherein the target comprises small biomolecules, proteins, or micro ribonucleic acids.
20. The microneedle of item 18 or 19, wherein the target comprises cortisol, vanomycin, gentamicin, tyrosinamide, thrombin, micro-RNA miR21, micro-RNA miR210, uric acid (UA), serotonin, insulin, adenosine triphosphate, or glucose.
21. The microneedle of any one of items 1 to 20, wherein the microneedle has a length of about 300 μm to about 1000 μm, such as about 800 μm.
22. The microneedle of any one of items 1 to 21, further comprising a conductive material.
23. The microneedle of item 25, wherein the conductive material comprises a metal nanoparticle, graphene-based material, conductive polymer, or an ionomer, or a combination thereof.
24. The microneedle of item 23, wherein the graphene-based material comprises ferrocene, graphene-oxide (GO) nanosheets, graphene-oxide (GO) nanoparticles, graphene-oxide (GO) nanocomposites, or a combination thereof.
25. The microneedle of item 23 or 24, wherein the conductive polymer, ionomer, or combination thereof comprises poly(3,4-ethylenedioxythiophene) polystyrene sulfonate; polyacetylene; polypyrrole; polyindole; polyaniline; a copolymer thereof; or a combination thereof.
26. A method of producing a microneedle, the method comprising: combining a functionalized hydrogel, a probe precursor, optionally a conductive material, and a crosslinking agent in a mold; and exposing the mixture in the mold to UV light to link at least a portion of the probe to the functionalized hydrogel and to form a crosslinked material.
27. The method of item 26, further comprising: removing the crosslinked material from the mold; and further exposing the unmolded crosslinked material to UV light.
28. The method of item 26 or 27, wherein combining the functionalized hydrogel, the probe precursor, optionally a conductive material, and the crosslinking agent in the mold comprises: dissolving about 50:1 to about 10:1 (wt/wt) of functionalized hydrogel:crosslinking agent in a buffer to form a functionalized hydrogel solution; optionally adding a conductive material to the functionalized hydrogel solution; optionally degassing the functionalized hydrogel solution; adding the functionalized hydrogel solution to the mold; partially drying the functionalized hydrogel solution in the mold; optionally, adding further functionalized hydrogel solution to the mold; adding the probe precursor to the mold; and optionally, drying the mixture in the mold further.
29. The method of any one of items 26 to 28, wherein the probe precursor comprises a solution of nucleic acid and a quencher.
30. The method of any one of items 26 to 29, wherein exposing the mixture in the mold to UV light comprises exposing the mixture to light of about 200 nm to about 400 nm, preferably about 360 nm light; and preferably for about 1 min to about 1 hour, such as about 10 to about 20 min.
31. The method of any one of items 26 to 30, wherein the hydrogel comprises hyaluronic acid; and functionalizing functionalizing the hydrogel comprises reacting hyaluronic acid with methacrylic anhydride to form methacrylated hyaluronic acid.
32. The method of any one of items 26 to 31, wherein the optional conductive material comprises metal nanoparticles, graphene-based material, such as ferrocene, graphene-oxide (GO) nanosheets, graphene-oxide (GO) nanoparticles, graphene-oxide (GO) nanocomposites, or a combination thereof; or a conductive polymer or ionomer, such as poly(3,4-ethylenedioxythiophene) polystyrene sulfonate, polyacetylene, polypyrrole, polyindole, polyaniline, or copolymers thereof.
33. The method of any one of items 26 to 32, wherein the mold is a negative polydimethylsiloxane mold.
34. An apparatus for detecting a target in a sample, the apparatus comprising: the microneedle according to any one of items 1 to 25; and a detector for detecting the measurable signal.
35. A transdermal patch comprising the microneedle according to any one of items 1 to 25.
36. A method for transdermal biosensing of a target in a subject, the method comprising: applying the transdermal patch according to item 35; detecting the measurable signal; and associating the measurable signal to the concentration of the target in the subject.
37. The method of item 36, wherein detecting the measurable signal is reagentless.
38. The method of item 36 or 37, wherein detecting the measurable signal comprises measuring the fluorescence intensity of the probe; or measuring an electrochemical signal.
39. The method of any one of items 36 to 38, wherein associating the measurable signal comprises comparing a measured intensity of the measurable signal to a calibration curve of measured intensities of known concentrations of the target.

To gain a better understanding of the invention described herein, the following examples are set forth. It should be understood that these examples are for illustrative purposes only. Therefore, they should not limit the scope of this invention in anyway

### EXAMPLES

### Example 1

### A Hydrogel Microneedle-assisted Biosensor Integrating Aptamer Probes and Fluorescence Detection for Reagentless Biomarker Quantification

1.1 Introduction

Herein, a fluorescent HMN biosensor based on methacrylated hyaluronic acid (MeHA) for on-needle and reagentless capture and detection of any biomarkers of interest is disclosed. This reagentless fluorescence assay for minimally invasive detection (RFMID) integrates a rapid and simple approach to link aptamer probes—short single-stranded DNA capable of specific binding to a target molecule—to the MeHA matrix. The application of this biosensor is demonstrated for ex vivo detection of adenosine triphosphate (ATP) and glucose, where HMN arrays functionalized with aptamer probes can detect the analyte concentrations with high sensitivity and specificity. It is also shown that the RFMID can be employed for tracking rising and falling levels of glucose in an animal model of diabetes. Specifically, the RFMID can accurately track severe hypoglycemia range, which cannot be detected using the commercially available glucose monitoring devices. The proposed RFMID technique is expected to pave the way for the next generation of real-time, continuous biosensors.

1.2 Results and Discussion

RFMID Detection Strategy

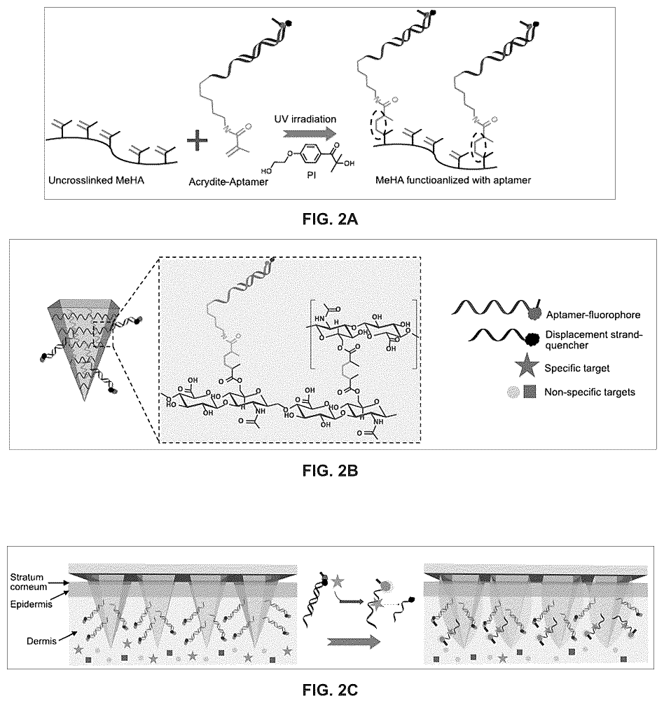

The RFMID integrates HMNs with aptamer probes as biorecognition elements to selectively capture the target analyte in a minimally invasive manner. HMNs are fabricated using MeHA, a highly swellable and biocompatible polymer that has been previously employed for enhanced ISF extraction and off-site target detection⁴. MeHA was synthesized by modifying HA with methacrylic anhydride^{4,21}. The degree of methacrylation was determined to be 20% by integration of methacrylate proton signals at 6.1, 5.7, and 1.8 ppm to the peak at 1.9 ppm related to the N-acetyl glucosamine of HA²² (see 1.5 Supplementary Information). An approach was employed to link aptamer probes into MeHA-HMNs by introducing an acrydite group to the proximal end of the aptamer. In the presence of a photoinitiator (PI) and under UV exposure, the acrydite group forms a covalent linkage with MeHA, enabling the attachment of aptamer probes to the HA network (FIGS. 2A and 2B). Additionally, a crosslinked hydrogel patch is formed via covalent attachment of unreacted MeHA carbon-carbon double bonds with the crosslinking agent.

For reagentless target detection, aptamer probes and a strand displacement strategy was used. Briefly, Cy3 fluorophore-conjugated aptamers were hybridzed with a DNA competitor strand that has been conjugated to a quencher (Dabcyl) molecule, called the quencher strand, and coupled the complex to MeHA through covalent linkage. The aptamer-quencher complex retains the fluorophore and quencher in proximity, producing no signal in the absence of a specific target. When the aptamer binds to the target, the quencher competitor strand dissociates and alleviates quenching of the fluorophore, producing a signal without requiring for any post-processing steps such as washing or adding a detection reagent. Despite that fact that the displacement-based analyses often suffer from low sensitivities, this is not the case in the presently described assay because the binding of an aptamer to its target molecule is more thermodynamically stable than the binding to its complementary stand. FIG. 2C shows a schematic of the RFMID reagentless detection principle. After fabrication, the RFMID is pressed through the skin for ISF extraction and target capture. Because non-specific binding of background chemicals does not cause dissociation of the quencher strand, the probe only responds to the binding of the specific target even when the background concentration is many orders of magnitude higher. This important feature enables the presently described sensor to operate in complex mediums such as ISF without the need for sample preparation or added reagents.

FIGS. 2A-2C are an overview of RFMID functionalization and sensing strategy. In FIG. 2A, the RFMID employs a covalent binding to attach aptamer probes into MeHA. The Cy3 conjugated aptamer with an acrydite group on the 5′ end is pre-hybridized with the corresponding competitor strand to form the aptamer-quencher complex, which can be covalently attached to the hydrogel matrix during the MeHA crosslinking process and in the presence of PI and under UV exposure. In FIG. 2B, the RFM ID's needles consist of crosslinked MeHA that is immobilized with aptamer-quencher complex. In FIG. 2C, the RFMID uses a reagentless process for target detection. Upon insertion, the RFMID penetrates through stratum corneum and epidermis and rapidly swells to extract transdermal ISF. During this process, fluorophore conjugated aptamer probes selectively bind to the specific target, leading to dissociation of the quencher strand and producing fluorescent signal.

RFMID Fabrication and Testing

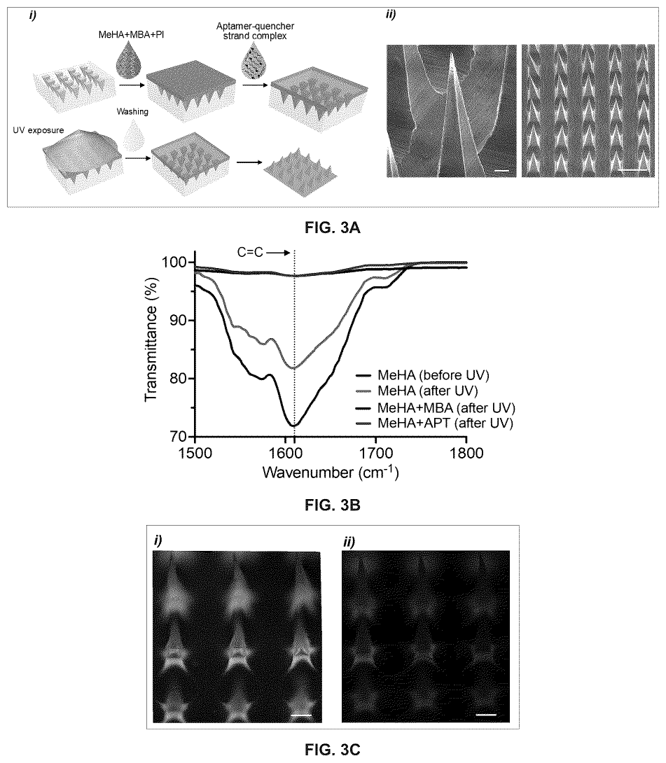

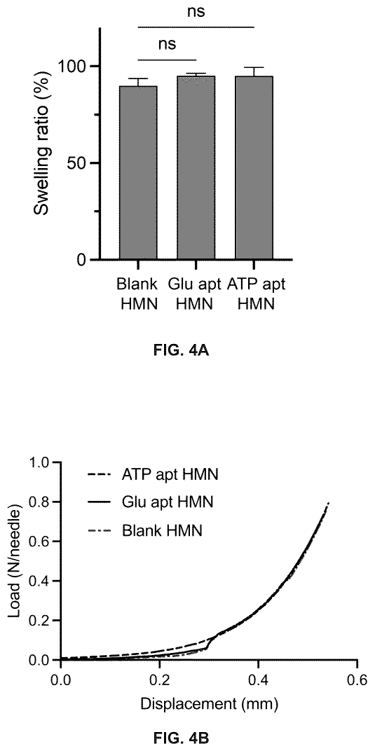

The RFMID was fabricated using a negative polydimethylsiloxane (PDMS) mold (FIG. 3A, i). MeHA, PI and the crosslinking agent, N,N′-methylenebisacrylamide (MBA), were dissolved in the corresponding buffer solution and applied to the PDMS mold.

FIGS. 3A-3C: show RFMID fabrication and functionalization. FIG. 3A is a schematic showing the fabrication process of RMFID: MeHA solution (40% w/v in Glucose or ATP binding buffer), PI, and MBA are casted into silicon mold, followed by the addition of aptamer-quencher complex. After drying, RFMID is crosslinked for 20 min under UV exposure and then washed to remove unbounded aptamer. RFMID device is then removed from the silicon mold and is ready to be applied for biomarker measurement. ii) Scanning electron microscopy (SEM) images showing the morphology of fabricated RFMID from the single needle view (left) and side view (right). Scale bar, 50 μm (left) and 500 μm (right). FIG. 3B is a FTIR spectra of MeHA thin films before and after UV irradiation and MeHA with MBA or with ATP aptamer probe after UV irradiation. The reduction in transmittance peaks of hydrocarbyl group at 1633 cm⁻¹, for crosslinked samples with aptamer probe or MBA shows the covalent attachment of the aptamer or MBA to the crosslinked patch. FIG. 3C is fluorescence microscopic images of RMFID patches functionalized with an aptamer probe only (left) and aptamer-quencher complex (right). Scale bar, 250 μm.

Before complete drying, the hybridized aptamer-quencher complex was added to the mold and the patches were left to dry. The patches were then exposed to UV for both aptamer linking and patch crosslinking. To remove the unbound aptamer probes, the patches were washed twice inside the mold to avoid the deformation of patch needles. The HMN patches were then removed from the mold and exposed to UV for another 5 min through the needle side to ensure formation of a crosslinked patch (not shown in FIG. 3A). Following this process, HMN patches were fabricated with sharp needles (FIG. 3A, ii) that are critical for effective skin penetration^{4,20}.

First, the chemical structure of hydrogel biosensor and efficiency of linking the aptamer probes to MeHA were investigated using Fourier-transform infrared spectroscopy (FTIR) (FIG. 3B). The spectrum of MeHA sample before UV exposure showed a strong peaks at ≈1633 cm⁻¹ (C═C)⁴, which significantly decrease in intensity after UV irradiation in MeHA sample with MBA, indicating that the carbon-carbon double bonds of MeHA were broken to form the covalent linkage with MBA. This reduction in the peak intensity was found to be minimal in the pure MeHA sample after UV irradiation. However, a similar trend with MeHA+MBA sample was observed for MeHA mixed with aptamer probes conjugated with acrydite group (MeHA+APT) after UV irradiation, suggesting a successful attachment of aptamer probes to the MeHA network. The successful aptamer linkage was also confirmed via fluorescence measurement after the fabrication process. The aptamer-quencher complex or only aptamer probes were applied and the patches were observed under fluorescence microscopy. The aptamer-only patch FIG. 3C, i) shows an elevated fluorescence signal compared to the patch functionalized with aptamer-quencher complex (FIG. 3C, ii), demonstrating both the successful aptamer linkage and quenching capabilities of Dabcyl.

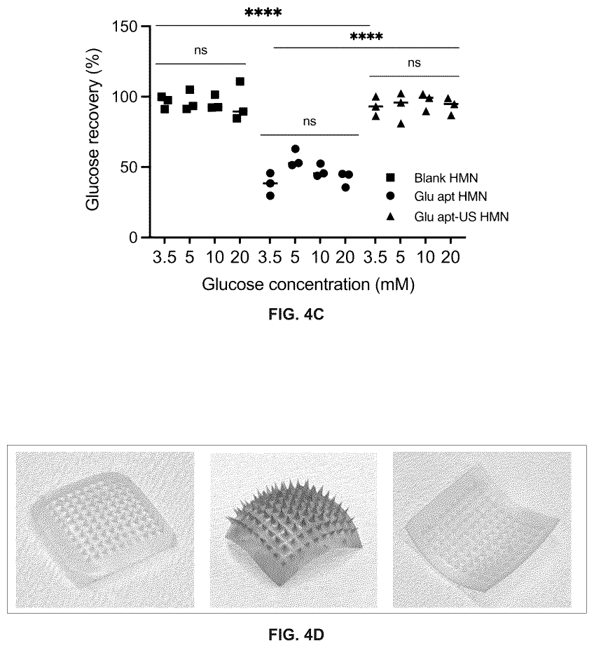

Next, the effect of aptamer probes on the swelling capability and mechanical strength of the RFMID patches was investigated. The swelling capability of the RFMID patches were tested by measuring the patch's weight before and after application through an agarose hydrogel for 10 mins. It was observed that the presence of aptamer probes does not have any significant effect on the swelling ability of MeHA hydrogel network (FIG. 4A). The slight increase in the swelling might be due to the lower crosslinking density in the RFMID patches (FIG. 2B). Also observed was a reduction of swelling by extending the crosslinking period (see 1.5 Supplementary Information), which agrees with previous work reported on MeHA-HMNs4. The mechanical strength of the MeHA-HMN patch was also evaluated through a compression test. HMN patches with and without aptamer show similar load versus displacement profiles (FIG. 4B), which indicates the mechanical strength is not influenced by the aptamer probes. The compression test result also shows that HMNs in this study can exert more than 0.6 N, which is sufficient for successful piercing through the skin⁴. The capability of the RFMID for target capture or recovery was investigated using glucose as a target molecule via testing three HMN samples; a blank HMN patch with no aptamer probes, a HMN patch functionalized with glucose aptamer probes (Glu apt HMN), and a HMN patch functionalized with glucose aptamer probes where upon target capture the aptamer probes were degraded using ultrasonication (Glu apt-US HMN)²⁶ (FIG. 4C). The three HMN patches were then pressed through an agarose hydrogel loaded with varying concentrations of glucose. The diffused or captured targets by blank HMN and Glu apt HMN were then recovered by centrifugation. For Glu apt-US HMN, prior to centrifugation, the patches were sonicated to degrade the aptamer and release the captured target. As shown in FIG. 4C, the blank HMN and Glu apt-US HMN show a high recovery rate where the absence and the degradation of aptamer probes, respectively, allow for the release and recovery of the target molecules. While in Glu apt HMN sample, the presence of aptamer probes hinders the full recovery of glucose.

This experiment ultimately indicates that the analytes diffuse into the patch needles and are captured by aptamer probes for subsequent detection. To visualize the target recovery capability, HMN patches were pressed through an agarose hydrogel loaded with Rhodamine B (RhoB) then the diffused dye was recovered (FIG. 4D and see 1.5 Supplementary Information).

FIGS. 4A-4D shows RMFID characterization. In FIG. 4A, the swelling ratio of a blank HMN patch without aptamer is compared to HMN patches functionalized with glucose or ATP aptamer probes. The presence of aptamer does not affect the swelling capability of HMN patches. Data are mean±s.d. n=3 repeated tests per group. The data among Glu apt HMN and ATP apt HMN groups was not significantly different from the blank HMN group (ns, not significant, P>0.9999 by the ordinary one-way analysis of variance (ANOVA) with Turkey's multiple comparison test). FIG. 4B shows the sechanical compression test for blank HMN and HMN patches functionalized with glucose and ATP aptamer probe. A compressive force in the longitudinal direction of the HMNs was generated by a moving transducer at a speed of 0.5 mm/min. In FIG. 4C, a blank HMN patch and HMN patches functionalized with the glucose aptamer probes (Glu apt HMN) were inserted into agarose hydrogels containing various concentrations of glucose (3.5, 5, 10, 20 mM) for 10 min to capture or recover glucose. For one group of aptamer HMNs, upon target capture, the aptamer probes were degraded by sonication (Glu apt-US HMN). The glucose was then recovered by centrifugation at 4,880 rpm for 5 min. Data is expressed as mean±s.d. n=3 replications per group. Data in Blank HMN and Glu apt-US HMN groups are significantly higher than Glu apt HMN group (****P<0.0001 by two-way ANOVA with Geisser-Greenhouse correction). Among each group, data of various glucose concentration group is not significantly different (ns, not significant, P>0.9999 by two-way ANOVA with Geisser-Greenhouse correction). FIG. 4D, shows optical images of a HMN patch before (left) after (middle) solution extraction from an agarose hydrogel containing 100 mg/mL RhoB, followed by the RhoB recovery (right). Scale bar, 5 mm.

In Vitro and Ex Vivo Detection of ATP and Glucose

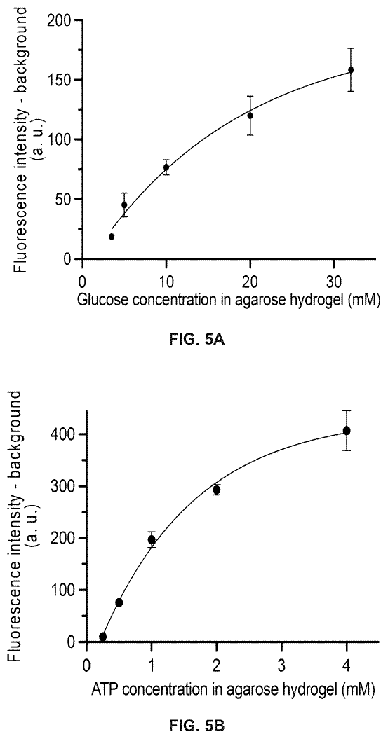

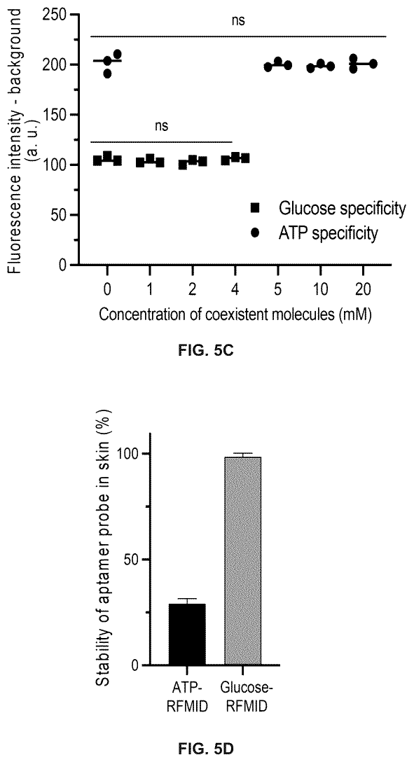

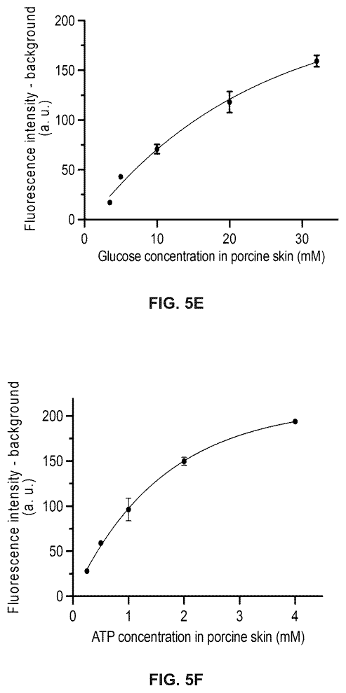

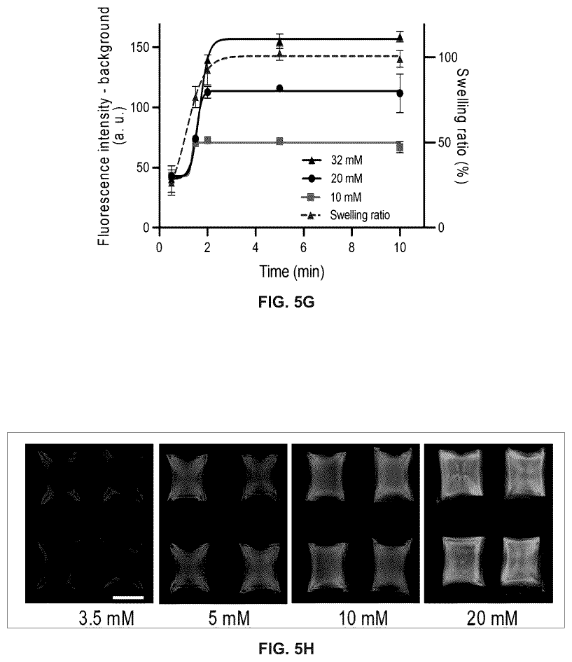

To investigate the sensor's capability for biomolecule detection, a series of experiments were conducted to measure varying concentrations of glucose (FIG. 5A) and ATP (FIG. 5B) in vitro using HMNs linked with glucose aptamer-quencher^{27,28} complex and ATP aptamer-quencher²⁹ complex, respectively. First the capability of the glucose/ATP aptamer-quencher strand complex for target detection was studied and the ratio of aptamer to quencher strand was optimized (see 1.5 Supplementary Information). HMN patches linked with the optimum ratio of aptamer and quencher strand were then applied on agarose hydrogels loaded with different concentrations of ATP or glucose for 10 min. The patch fluorescence intensity was measured before and after application to agarose hydrogel and it was observed that by increasing the target concentration, the signal intensity increases, demonstrating the target capture and dissociation of the quencher strand. The limit of detection (LOD) of the biosensor was estimated to be three times the standard deviation of the fluorescence signal intensity from a blank agarose hydrogel (See 1.4 Methods). The HMN sensor achieved a LOD of 2.5 mM for glucose and 0.2 mM for ATP measurements. To further explore the sensitivity and reliability of detection, cross-reactivity among glucose and ATP was examined by applying HMN patches functionalized with glucose (ATP) aptamer into an agarose hydrogel loaded with a definite concentration of glucose (ATP) while the concentrations of ATP (glucose) increased. It was observed that for both glucose and ATP, the changes of the fluorescence intensity for the non-specific target were almost unnoticeable, indicating the high selectivity of the sensor (FIG. 5C).

Upon successful detection of target analytes in agarose hydrogel, the sensor capability for in situ biomarker measurement was tested using an ex vivo skin model. First, the degradation of both ATP and glucose aptamer probes via nucleases present in the skin was examined. To this end, functionalized HMN patches with only aptamer probes were applied through a blank porcine skin or agarose hydrogel (with no nucleases present). It was observed that the fluorescence intensity of the patches did not change after insertion. After penetration, the quencher strand was added to the HMN patches and a reduction in fluorescence signal was observed. The reduction of fluorescence signal intensity of the patches applied to skin or agarose were then measured and compared. The fluorescence signal reduction in HMNs penetrated through skin was 98% and 29% of the ones inserted into blank agarose hydrogel for glucose and ATP aptamer probes, respectively, (FIG. 5D) indicating that most of ATP aptamers became degraded in the skin and could not be hybridized to the corresponded quencher strands. These results confirm that the glucose aptamer has a great stability in skin while the ATP aptamer might not be sufficiently stable to permit target detection in a complex ISF environment.

The RFMID was then employed for glucose (FIG. 5E) and ATP (FIG. 5F) detection using porcine skin equilibrated with different concentrations of the target analytes. Microneedle traces were evident in the porcine skin, showing an efficient skin penetration (see 1.5 Supplementary Information). These data were used to construct standard curves that correlate fluorescence signal intensity to glucose or ATP concentration in skin ISF. The presently described device achieved a LOD of 1.1 mM for glucose and 0.1 mM for ATP measurements in skin ISF. The glucose detection limit and dynamic range cover the clinical hypoglycemia, euglycemia and hyperglycemic ranges. This system can potentially be employed to reliably quantify pre- and post-prandial glucose concentrations in patients with diabetes while the accuracy of most of commercially available glucose monitoring devices is still the lowest within the hypoglycemic range.

To determine the shortest timescale for effective capture of target analytes, glucose-RFMID patches were applied on the porcine skin equilibrated with various concentrations of glucose for different durations. 2 min of microneedle patch administration was found to be sufficient to capture glucose (FIG. 5G) and longer administration (5 and 10 min) did not change the measured fluorescence. Without wishing to be bound by any particular theory, it is suspected that the fast response is because of the swelling characteristic of MeHA-HMNs that can reach their maximum swelling within 2 min, enabling increased and rapid ISF extraction and thus target capture. The 2 min response time is also independent of the target concentration and increased glucose concentration does not affect the time needed for the target molecules to diffuse into the patch needles. Previously reported MN-based diagnostic devices require at least 140 min to detect the target analytes, including the time needed for MN application, washing, and adding the detection agents.^{37,38} FIGS. 5H and 5I show that the fluorescence intensity of MN patches increases with the rising glucose and ATP concentration, respectively, confirming elevated dissociation of quencher strand and target capture.

FIGS. 5A-5H shows in vitro and ex vivo detection of ATP and glucose. FIG. 5A, shows the RFMID devices functionalized with glucose and in FIG. 5B, ATP aptamer-quencher complex was applied into agarose hydrogels containing varying concentrations of glucose (3.5, 5, 10, 20, 32 mM) or ATP for 10 mins (0.25, 0.5, 1, 2, 4 mM). The fluorescence signal intensity was measured before and after applying the patches and the difference in signal intensity was reported. In FIG. 5C, the specificity of RFMID devices for capturing the specific target was tested. The ATP-RFMID devices were applied into agarose hydrogels containing 1 mM of ATP while glucose concentrations increased (0, 5, 10, 20 mM). Similarly, the glucose-RFMID devices were applied into agarose hydrogel containing 20 mM of glucose while ATP concentrations increased (0, 1, 2, 4 mM). FIG. 5D shows the stability of ATP and glucose aptamer probes in skin. HMN patches functionalized with only aptamer probes were applied to porcine skin or a blank agarose hydrogel. Upon insertion, the corresponding quencher strands were added to the patches. The reduction of fluorescence signal intensity of the patches applied to skin or agarose were then measured and compared. The graph shows 1−[(fa−fs)/fa] as a measure of aptamer stability, where fa and fs are the reduction in fluorescence signal for HMN patches applied into the agarose hydrogel or skin, respectively, upon addition of quencher strands. The RFMID devices functionalized with glucose (FIG. 5E) or ATP (FIG. 5F) aptamer-quencher complex were applied into porcine skin equilibrated with varying concentration of glucose (3.5, 5, 10, 20, 32 mM) or ATP (0.25, 0.5, 1, 2, 4 mM) for 5 min. The fluorescence signal intensity was measured before and after applying the patches and the difference in signal intensity was reported. Since some ATP aptamer probes are degraded in skin (based on experiments in FIG. 5D), the level of fluorescence signal is less compared with agarose hydrogel experiment in FIG. 5B. In FIG. 5G, Glucose-RFMID devices were applied through porcine skin equilibrated with different glucose concentrations (10, 20, and 32 mM) for different durations. The swelling of patches and the fluorescence response were then measured. 2 min was found to be the optimal time of HMN application. FIG. 5H shows fluorescent microscopic images of glucose-RFMID and FIG. 51 shows ATP-RFMID after capturing varying concentrations of ATP (0.5, 1, 2, 4 mM) or glucose (3.5, 5, 10, 20, 32 mM) loaded in the agarose hydrogel. Data are presented as mean+s.d. a.u., arbitrary units.

In Vivo Glucose Detection in Animal Models of Diabetes

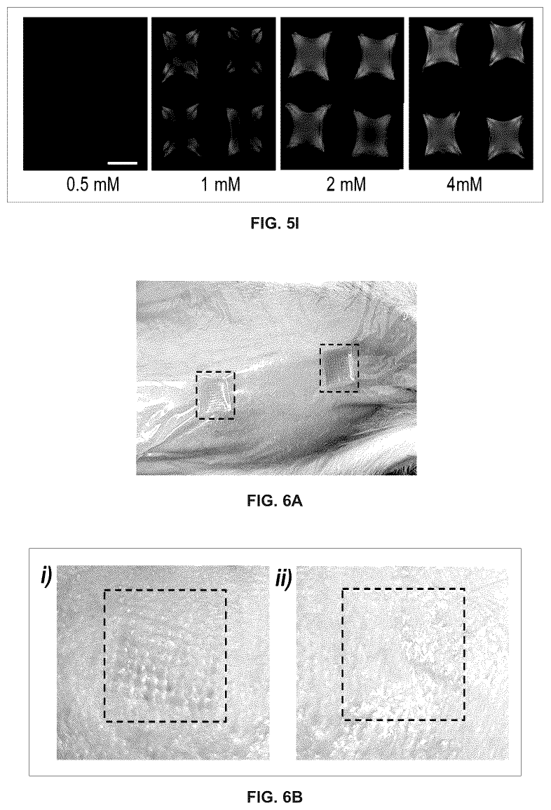

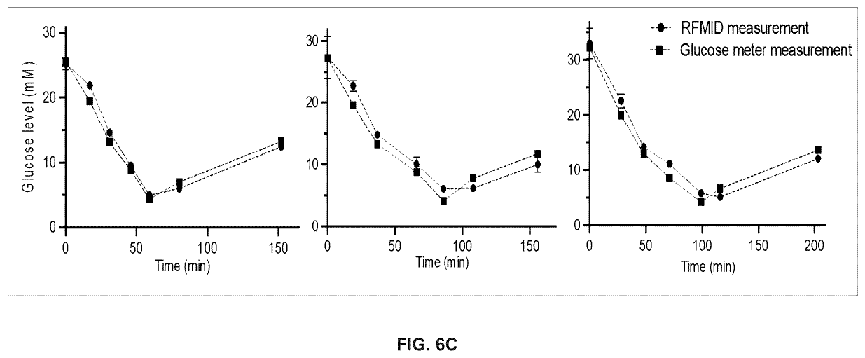

Having demonstrated the platform's ability to detect glucose in vitro and ex vivo sensitively and accurately, its performance in vivo was evaluated using a streptozotocin-induced rat model of diabetes. Prior to the animal experiment, the cytotoxicity of the composite materials was evaluated in NIH-3T3 fibroblast cells using MTT assay (see 1.5 Supplementary Information). Results showed that NIH-3T3 fibroblast viability was not significantly influenced, suggesting that the RFMID materials were biocompatible. RFMID patches were inserted on the rat's dorsal skin side (FIG. 6A). HMN patches efficiently penetrated the tissue as evidenced by the microneedle traces and the skin recovered well post-treatment (FIG. 6B). The HMN punctures disappeared gradually within 15 min of removing the patch. This is due to the biocompatibility and minimal invasiveness of the RFMID patches. The diabetic rats were fasted for 5 hours prior to the experiment and treated first with 4 IU kg⁻¹ dose of human recombinant insulin subcutaneously to lower blood glucose and after hypoglycemia was reached with 30% glucose intraperitoneally to increase blood glucose again.

The RFMID patches for glucose detection were applied at different time points and kept on the skin for 5 min (FIG. 6C and see 1.5 Supplementary Information). These results demonstrate that the RFMID device can track the falling and rising glucose concentrations in animal models. In parallel, the RFMID results were compared with conventional glucose measurements from a handheld glucose monitor using blood samples collected from the rat tail vein and observed that both sets of results correlated well with a time lag of 3-14 mins. This time lag is attributed to the locations of glucose and differing transport efficiencies between ISF and circulating blood. Patients with diabetes also showed a time lag of 4-10 min in the change of ISF glucose levels relative to blood glucose concentrations³¹. The differences between glucose responses in individual rats clearly show the inter-individual variability, even under controlled conditions with genetically similar animals. This has important implications for human patients, who are genetically diverse and exposed to different environmental conditions. Thus, these results highlight the necessity of personalized glucose monitoring using a simple and reagentless approach.

FIGS. 6A-6C shows in vivo glucose detection in diabetic rats. In FIG. 6A, RFMID patches were applied into the dorsal skin of awake rats and fixed with Tegaderm tapes for 5 min. FIG. 6B, shows magnified images of the trace of a patch on the skin (i). The skin is recovered after 15 min (ii). FIG. 6C shows RFMID measurement of glucose levels in three different diabetic rats over 150-200 mins. The rats were injected with 4 IU kg⁻¹ dose of human recombinant insulin (at t=5 min; after baseline measurement) subcutaneously or 30% glucose solution (at t=63, 93, 109 min for different rats and after reaching hypoglycemia condition) intraperitoneally to reduce or increase the blood glucose level, respectively. Six ranges of glucose levels were targeted in total: TO: 25-35 mM, T1: 15-25 mM, T2: 10-15 mM, T3: 5-10 mM, T4: 3-10 mM, and T5>10 mM. After reaching each range, three RFMID patches were applied to the dorsal skin of rats for 5 mins. For each timepoint, blood samples were collected from the tail vein before and after applying RFMID patches, measured the glucose levels using a hand-held glucose meter, and reported the average to compare with RFMID measurement. These results correlated closely with a time lag of 3-14 mins and highlight the inter-individual variability in the insulin or glucose response.

1.3 Conclusions

Herein is demonstrated the first technology to combine HMN arrays with aptamer probes to summon their merits for reagentless and minimally invasive target detection. A comprehensive characterization has been shown for HMN patches functionalized with aptamer probes where addition of the aptamer probes did not have a significant effect on the swelling ability or mechanical strength of the patches. Experiments in skin specimens equilibrated with varying concentrations of glucose or ATP indicate that the sensor has high sensitivity and specificity to detect clinically relevant concentrations of both analytes, with a LOD of 1.1 mM for glucose and 0.1 mM for ATP. In vivo experiments in awake diabetic rats confirmed the RFMID ability to measure changes in glucose with no need for adding any reagents and highlighted the RFMID platform's capacity to detect inter-individual variations in glucose response between animals—a critical feature for clinical implementation. Importantly, RFMID measurements closely matched those obtained with standard clinical glucose sensors.

Based on the above experimental results, it was considered that RFMID device can be fabricated using any other polymer with C═C for probe binding. The probe can be either aptamer or antibody with appropriate functional group to bind to the hydrogel network.

It is contemplated that this system could be modified for continuous, real-time measurement in a minimally invasive manner. Aptamer probes have been employed to the continuously measure biomolecules in vivo using electrochemical sensors where the structure switching characteristics of aptamers were integrated with a redox reporter to produce a concurrent electrochemical signal. The aptamer-based electrochemical detection has been applied for continuous measurement of different metabolites and drugs, however, the complicated and invasive design (i.e., insertion into the vein that requires surgery) and/or the short monitoring capacity (a few hours) are key shortcomings. The presently disclosed microneedles can be deployed as an integrated technology to continuously collect individual patient molecular profiles in a minimally invasive manner, allowing continuous and prolonged measurement of any targets of interest, such as drugs with narrow therapeutic range.

Finally, it is to be emphasized that the RFMID system is a platform that could be readily modified to measure other circulating analytes in vivo, for which aptamers pairs are available, thus making it potentially a versatile tool for diverse biomedical applications.

1.4 Methods

Materials

The Pharma-grade sodium Hyaluronic acid (HA, MW 300 KDA) was purchased from Bloomage Co., Ltd (China). 1×PBS, Dimethyl sulfoxide (DMSO, 25-950-CQC), was purchased from Corning, USA. Irgacure 2959 (photo initiator, PI), N′-methylenebisacrylamide (MBA), methacrylic anhydride (MA), glucose and ATP solution and other chemicals were purchased from Sigma Aldrich (Canada). 100 mM ATP solution (R0441) was purchased from Thermo Fisher. The porcine ear skin was obtained from a local supermarket. All the aptamer and displacement strand were purchased from Integrated DNA technologies. Sequence of ATP²⁹ and glucose²⁸ aptamer and competitor strands were obtained from literature. Sequence and modification of all aptamer and competitor strands are indicated in Table 2.

TABLE 2
Sequence and modification of aptamer and competitor strands

| | | Internal | | |
|---|---|---|---|---|
| Name | 5′ mod | mod | 3′ mod | Sequence |
| Glucose aptamer | 5ACryd | iCy3 | | CTCTCGGGACGACCGTGTGTGTTGCTCTGTAACAGTGTCCA |
| | | | | TTGTCGTCCC; SEQ ID NO. 13 |
| | | | | (Acrydite-Cy3-CTCTCGGGACGACCGTGTGTGTTGCTC |
| | | | | TGTAACAGTGTCCATTGTCGTCCC; SEQ ID NO. 13) |
| Glucose competitor | | | 3DAb | GGTCGTCCCGAGAG; SEQ ID NO. 14 |
| | | | | (GGTCGTCCCGAGAG-Dabcyl; SEQ ID NO. 14) |
| ATP aptamer | 5ACryd | iCy3 | | CACCTGGGGGAGTATTGCGGAGGAAGG; |
| | | | | SEQ ID NO. 15 |
| | | | | (Acrydite-Cy3-CACCTGGGGGAGTATTGCGGAGGAAG |
| | | | | G; SEQ ID NO. 15) |
| ATP competitor | | | 3DAb | TTTTCCAGGTG; SEQ ID NO. 16 |
| | | | | (TTTTCCAGGTG-Dabcyl; SEQ ID NO. 16) |

Acryd-acrydite modification
Cy3-Cyanine 3 modification
Dab-Dabcyl modification

Synthesis and Characterization of MeHA

MeHA was synthesized based on the modified protocol established by Poldervaart et al²¹. 2.0 g HA was dissolved in 100 mL Millipore water and stirred overnight under 4 degree for complete dissolving. Subsequently, 1.6 mL MA was added into HA solution and 3.6 mL of 5N NaOH solution was added to adjust the solution to pH 8-9. The mixture was stirred overnight under 4 degree to complete the reaction. Next, MeHA was precipitated by acetone and washed three times with ethanol. Subsequently, precipitated MeHA was redissolved in Millipore water and was dialyzed for 2 days to remove the impurity. The purified MeHA was lyophilized for 3 days. Eventually, 2 -5 mg of MeHA was dissolved in 1 mL Deuterium oxide (D₂O) (Sigma Aldrich, 151882) and then tested with 300 MHz ¹HNMR with 10 ms time scale. The degree of methacrylate modification was determined by integration of methacrylate proton signals at 6.1, 5.7, and 1.8 ppm to the peak at 1.9 ppm related to the N-acetyl glucosamine of HA²².

Fabrication of RMFID

For each RMFID patch, 50 mg MeHA, 1 mg PI and 1 mg of MBA were dissolved in 1.25 mL of glucose or ATP aptamer binding buffer. The MeHA solution was then sonicated for 5 min to remove the bubbles. Subsequently, 0.5mL of MeHA solution was deposited on a negative polydimethylsiloxane (PDMS) mold (Micropoint, Singapore), and degassed for 90 s.

After drying at room temperature for 5 hours, another 0.75 mL of MeHA solution was casted on the mold followed by drying at room temperature for 10 hours. Next, 10 μL of aptamer-quencher strand solution composed of 1 μM aptamer and 10 μM corresponding quencher strand with 15 min pre-hybridization was loaded on the HMN followed by drying at 45 degrees for 30 min. Dry HMN patches were then crosslinked by UV light with 360 nm wavelength for 15 min. The RMFID patches were washed twice with 10 μM of glucose or ATP aptamer binding buffer and dried under 45 degrees. Last, MN patches were carefully separated from PDMS molds and further crosslinked for 5 mins. After being trimmed, the RMFID patches were observed under a fluorescence microscope (Nikon, Ti2). The fabricated needles of the RMFID patch were 850 μm in height, 250 μm in base width and 500 in internal spacing.

Chemical Characterization

The Fourier-transform infrared spectroscopy (FTIR, Bruker Hyperion 3000 FTIR Microscope) was conducted to study the crosslinked degree of HMNs and aptamer functionalization efficiency. The following four samples were made in 1 mL DI water: two samples of 50 mg/mL MeHA solution containing 1 mg/mL photo initiator (MeHA); one sample of 50 mg/mL MeHA solution containing 1 mg/mL photo initiator and 1 mg/mL MBA (MeHA+MBA); and one sample of 50 mg/mL MeHA solution containing 1 mg/mL photo initiator and 1 μM aptamer (MeHA+APT). One sample of MeHA, MeHA+MBA and MeHA+APT were crosslinked under UV exposure for 20 min, and then their FTIR spectrum is recorded from 4000 to 400 cm⁻¹ and were compared with not-crosslinked MeHA sample.

Mechanical Test and Skin Penetration Efficiency

The RMFID patches were applied on rat dorsal skin or on the porcine skin for 15 min. Subsequently, the trace on skin was recorded by digital camera every 5 min for 15 mins. The mechanical strength of MN patches was measured using Instron 5548 micro tester equipped with 500N compression loading cell. For each test, the HMN patch was placed flat on its backside (tips facing upwards) on a compression platen. The distance between two platens was set to 1.5 mm. A vertical force was applied (at a constant speed of 0.5 mm/min) by the other platen. The compression loading cell capacity was set to 70 N. The load (force; N) and displacement (distance; mm) was recorded by the testing machine every 0.1 s to create the load-displacement curve.

In Vitro Cytotoxicity Assay (Evaluation of Biocompatibility)

The biocompatibility of RMFID was investigated using mouse fibroblast cells (NIH-3T3). Cells were seeded at a density of 50,000 cells per well in a 96-well plate with a final volume of 100 μL. Subsequently, cells were exposed to 10μL of sample solution for 24 hours. The 5mL sample solution contains 50 mg MeHA, 1 mg MBA, 1 mg photo initiator and 10 μL of 1 μM ATP or glucose aptamer solution. 10 μL of DMEM medium solution was used as control. After sample exposure, 10 A of the 5 mg/ml Methylthiazolyldiphenyl-tetrazolium bromide (MTT) (Sigma Aldrich, M5655) solution was added to all wells. Next, the plate was incubated in the absence of light for 3 hours. 150 μI of DMSO was added and gently pipetted to all wells to break up cells and release the formazan crystals. The absorbance of the samples was then obtained at 540 nm using a spectrophotometer.

Swelling Studies

A 1.4 wt % agarose (Sigma Aldrich, A0169) hydrogel was prepared in DI water. The dry mass (W₀) of HMNs were measured before applying through agarose. Then the HMNs were penetrated to the agarose through a layer of parafilm and swelled for 10 min. Next, wet mass (Wₜ) of the swelled HMN patches was measured. The swelling ratio of HMNs was calculated based on the below formula:

$$swelling ratio = \frac{{W}_{t} - {W}_{0}}{{W}_{0}} \times 100 \%$$

Assessing Rhodamine B Recovery Rate

A 1.4wt % agarose hydrogel containing 100 mg/mL (Co) Rhodamine B (Rho B) (Sigma Aldrich, R6626) was prepared. HMN patches with crosslink time of 5, 10, 15, 20 min were weighted, and their dry mass (W₀) was recorded. Next, HMNs were punched into RhoB agarose through parafilm for 10 min. After measuring the wet mass (Wₜ), the swelled HMN was mixed with 300 μL (V) Millipore water in a centrifuge tube followed by a 5 min centrifugation at 10 K rpm⁴. Subsequently, 80 μL of recovered solution was transferred into a 96-well plate to measure the absorbance at 552 nm. The Rho B recovery rate were calculated based on the following formula.

$$RhoB recovery rate = \frac{{C}_{t} \times V}{{C}_{0} \times ( {W}_{t} \times {W}_{0} ) \div ρ} \times 100 \%$$

In the equation, C₀ refers to the initial RhoB concentration (100 mg/mL), Cₜ is the detected RhoB concentration recovered from MN, V is the volume of recovered solution (300 μL), and (Wₜ−W₀)÷ρ is the volume of solution absorbed by MN.

Assessing Glucose Recovery Rate

To evaluate the capability of the RFMID for glucose recovery, three groups of samples were prepared: a group of blank HMN patches without aptamer probe functionalization and two groups of HMN patches functionalized with glucose aptamer probes (Glu apt HMN). After measuring the dry mass (W₀), HMNs were penetrated to 1.4 wt % agarose containing varying glucose concentration of 3.5, 5, 10, 20 mM for 10 min. After measuring the wet mass (Wₜ), one group of HMN patches functionalized with aptamer probes was sonicated for 10 min and named as Glu apt-US HMN. Subsequently, all the HMN were mixed with 300 μL (V) Millipore water in a centrifuge tube followed by 5 min centrifugation at 2,100 rcf. Subsequently, 250 μL of recovered solution was transferred into a 96-well plate and the recovered glucose concentration was measured using a glucose (GO) assay kit purchased from Sigma (GAGO20). Glucose recovery rate was defined by the following formula.

$$Glucose recovery rate = \frac{{C}_{t} \times V}{{C}_{0} \times ( {W}_{t} \times {W}_{0} ) \div ρ} \times 100 \%$$

C₀ refers to the initial glucose concentrations (3.5, 5, 10, 20 mM), Cₜ is the detected glucose concentration recovered from MN, V is the volume of recovered solution (300 μL), (Wₜ−W₀)÷ρ is the volume of solution absorbed by HMN.

In Vitro Glucose and ATP Measurement

The Fluorescence intensity (FI) of the RFMID was recorded by the fluorescent microscope from the base side. The RFMID patches with ATP or glucose aptamer probe were applied on 1.4 wt % agarose hydrogels containing various concentrations of ATP (0, 0.25, 0.5, 1, 2, 4 mM) or glucose (0, 3.5, 5, 10, 20, 32 mM) for 10 min, respectively. Next, the fluorescent 5 intensity of the RFMID after target detection was recorded. Finally, the corresponding needles were identified and the fluorescence intensity difference before and after target capturing were measured and calculated by subtracting FI before target capture from the FI after target capture.

Assessing Specificity of RMFID

The RMFID functionalized with glucose or ATP aptamer probe was punched into the 1.4 wt % agarose containing 10 mM glucose and 0, 1, 2, 4 mM ATP, or 1 mM ATP and 0, 5, 10, 20 mM glucose for 10 min, respectively. The fluorescence intensity of RMFID before and after being applied on agarose was recorded by fluorescent microscope.

Ex Vivo Glucose and ATP Measurement

After being rinsed with DI water and trimmed to 1 cm by 1 cm square, porcine ear skins were equilibrated in 1×PBS with various concentrations of ATP (0, 0.25, 0.5, 1, 2, 4 mM) or glucose (0, 3.5, 5, 10, 20, 32 mM) overnight. Subsequently, fluorescent intensity RFMID patches were recorded with the fluorescence microscope from the base side. Next, ATP or glucose RFMID patches were applied on porcine skin equilibrated with ATP or glucose for 5 min, respectively. Tegaderm tape (3M) was used to fix RFMID patches on the skin. After drying under room temperature, the RFMID patches were observed under the fluorescence microscope and their fluorescent intensity was recorded. Similar to the in vitro experiment, the fluorescent intensity difference of RFMID before and after target capturing was calculated.

In Vivo Glucose Measurement in Diabetic Rats

Animal studies were performed in accordance with the Guidelines for the Care and Use of Laboratory Animals and the Animal Welfare Act Regulations; all protocols were approved by the University of Toronto Institutional Animal Care and Use Committee. An established model of T1D, the streptozotocin (STZ)-induced diabetic rat, was used to explore the in vivo performance of RFMID. Male Sprague Dawley rats (Charles River, 100-150 gr) were injected with STZ (65 mg/kg i.p.) that destroys the host's pancreatic beta-cells secreting insulin³⁵. After the STZ injection, the rats were monitored for 1 week and their blood glucose was measured every 2 days using a glucose meter (OneTouch® Ultra®, LifeScan, Inc., USA). Diabetic rats with blood sugar stabilized above 17 mM were selected for this study. Before starting experiments, the rats were fasted for 5 hours. Rat skin was then shaven, treated with hair removal cream, and dried prior to MN patch application. RFMID patches for ISF glucose detection were prepared and their fluorescence intensity was measured. The baseline blood and ISF glucose level of rats were measured by glucometer and RFMID, respectively. Subsequently, 4 units of insulin were injected to the rats subcutaneously and blood glucose levels were tracked by glucometer every 5 min. The RFMID patches were applied on rats' skin and fixed with Tegaderm tape for 5 min, when blood glucose level decreased to certain ranges (5 ranges in total): T0: 25-35 mM, T1: 15-25 mM, T2: 10-15 mM, T3: 5-10 mM, T4: 3-5 mM. After reaching to hypoglycemia regime, 0.5 mL of 30% glucose solution was intraperitoneally injected into the rats, followed by another two time-point ISF glucose measurements by RFMID. After drying at room temperature, RFMID patches were observed under the microscope and their fluorescence intensity was recorded. Rats' ISF glucose levels were calculated by interpolating the fluorescence intensity difference before and after detection into the ex vivo RFMID glucose detection calibration curve.

Statistical Analysis

All the statistical analysis was conducted using GraphPad Prism 9. The statistical difference between groups in biocompatibility test, RFMID specificity test and swelling experiment was analyzed using ordinary one-way ANOVA with Tukey's multiple comparison test. In glucose recovery experiment, the statistical difference between different HMN groups and various glucose concentrations were analyzed using two-way ANOVA with Geisser-Greenhouse correction. The significance of statical difference was calculated with 95% confidence interval (P<0.05) and shown in GP style (0.1234 (ns), 0.0332 (*), 0.0021 (**), 25 0.0002 (***), <0.0001 (****)) in graphs. Each experiment contains three parallel replicates. All data is expressed as mean±s.d. For in vitro and ex vivo glucose and ATP measurement, fluorescence signal raw data after subtracting the background signal was used to calculate the calibration curves using the sigmoidal 4PL nonlinear regression model. The LOD is defined as the minimum target concentration that can be detected by the presently described RFMID 30 device and calculated by interpolating the mean fluorescence signal of control group plus three times of s.d. into the corresponding calibration curve.

1.5 Supplementary Information

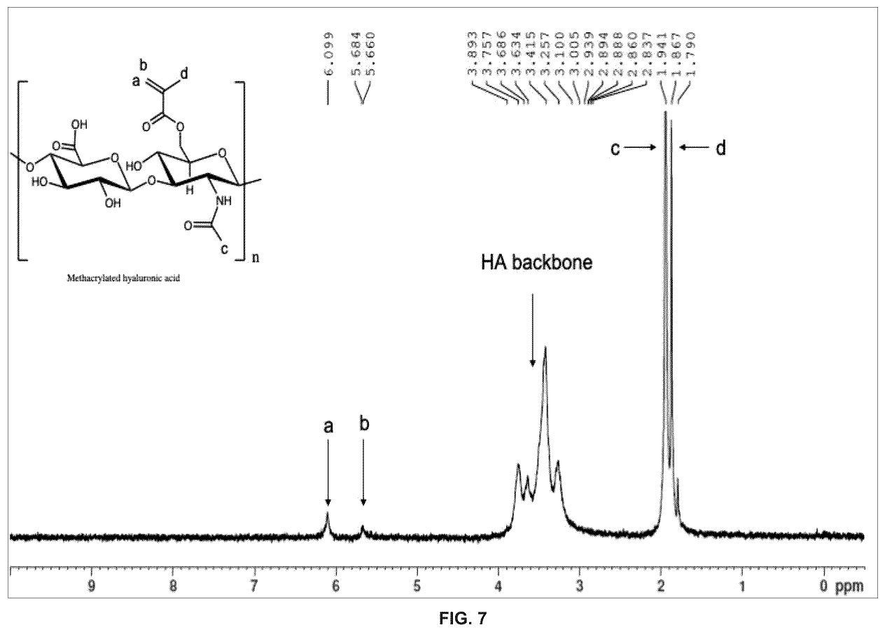

FIG. 7 shows 1H NMR spectra of MeHA. MeHA was characterized with 300MHz 1HNMR with 10ms time scale to determine the degree of methacrylate modification.

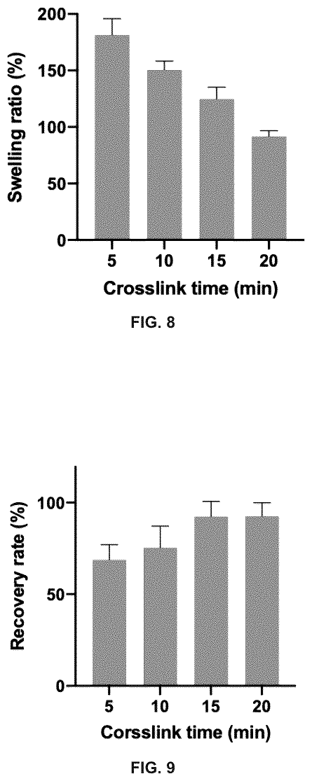

FIG. 8 demonstrates characterization of crosslinked MeHA-HMN. MeHA-HMNs with various crosslinking time (5, 10, 15, 20 min) were characterized based on their swelling ability.

FIG. 9 demonstrates characterization of crosslinked MeHA-HMN. MeHA-HMNs with various crosslinking time (5, 10, 15, 20 min) were characterized based on their Rhodamine B recovery rate.

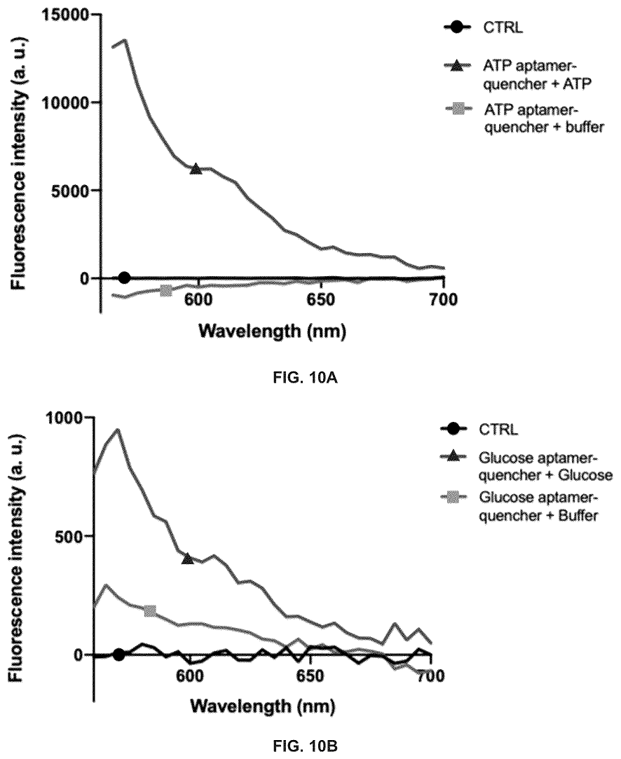

FIG. 10A and FIG. 10B is an assessment of aptamer binding efficiency. The target binding efficiency of A) ATP and B) glucose aptamer was tested with spectrophotometer. A spectral scanning measurement was conducted with the excitation of 530 nm, and an emission from 550 nm to 700 nm. Buffer solution was used as background control (CTRL). The aptamer probe was hybridized to its corresponding quencher for 30 min before adding the targets. Subsequently, the fluorescence intensity difference was measured after capturing target for 30 min.

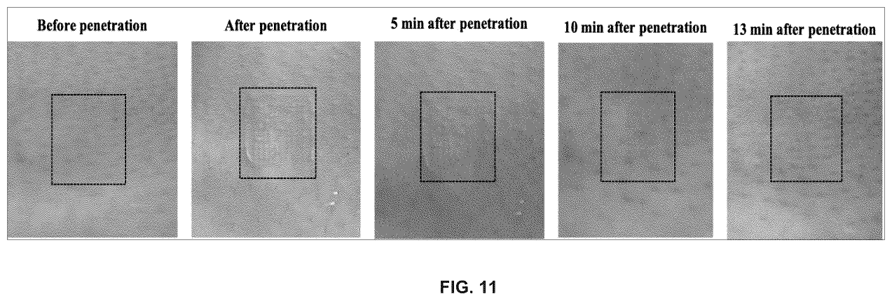

FIG. 11 is an ex vivo porcine skin penetration efficiency test using RFMID. Glucose RFMID patches were applied to the porcine ear skin for 5 min with index finger pressure. After removing the RFMID patches, skin samples were imaged with digital camera for 13 min with a 5 min interval to observe the micro-sized holes left by RFMID.

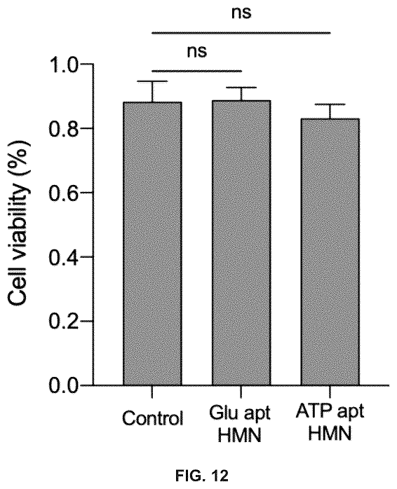

FIG. 12 shows a biocompatibility test of aptamer MeHA HMN. Mouse fibroblast cells were cultured at 100,000 cells per well in a 96-well plate and exposed to 10 μL of MeHA samples solution for 24 hours. Sample solution composed of 50 mg MeHA, 1 mg MBA, 1 mg photo initiator and 10 μL of 1 μM ATP or glucose aptamer solution. Cells in control group were exposed to cell culture media. Each group has three replicates. Subsequently, cell viability was detected with MTT assay.

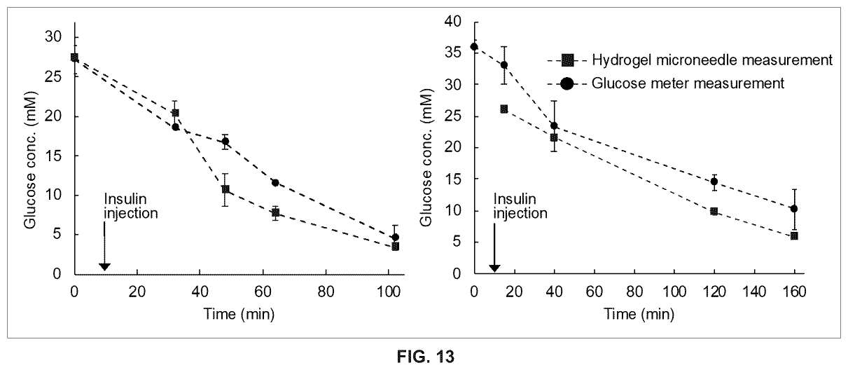

FIG. 13 demonstrates a glucose measurement in live diabetic animals using RFMID. Insulin bolus was injected after the baseline measurement which results in decrease in the glucose level to hypoglycemia regime.

1.5.1 Further Supplementary Information

Materials

The Pharma-grade sodium Hyaluronic acid (HA, MW 300 KDA) was purchased from Bloomage Co., Ltd (China). 1×PBS, Dimethyl sulfoxide (DMSO, 25-950-CQC), was purchased from Corning, USA. Irgacure 2959 (photo initiator, PI), N′-methylenebisacrylamide (MBA), methacrylic anhydride (MA), glucose, L-tyrosinamide (T3879), and ATP solution and other chemicals were purchased from Sigma Aldrich (Canada). 100 mM ATP solution (R0441) and 5.7 mg/mL human alpha-thrombin native protein (RP-43100) were purchased from Thermo Fisher (Canada). The porcine ear skin was obtained from a local supermarket. All the aptamer and displacement strand were purchased from Integrated DNA technologies (IDT). Sequence of ATP[29] and glucose[40] aptamer and competitor strands were obtained from literature. Sequence and modification of all aptamer and competitor strands are indicated in Table 3.

Fabrication of RMFID

For each RMFID patch, 50 mg MeHA, 1 mg PI and 1 mg of MBA were dissolved in 1.25 mL of glucose or ATP aptamer binding buffer. The MeHA solution was then sonicated for 5 min to remove the bubbles. Subsequently, 0.5mL of MeHA solution was deposited on a negative polydimethylsiloxane (PDMS) mold (Micropoint, Singapore), and degassed for 90 s. After drying at room temperature for 5 hours, another 0.75 mL of MeHA solution was casted on the mold followed by drying at room temperature for 10 hrs. Next, 10 μL of aptamer-quencher strand solution composed of 1 μM glucose or ATP aptamer and 10 μM corresponding quencher strand, 2 μM L-tyrosinamide aptamer and 2.5 μM quencher strand or 1 uM of thrombin aptamer-quencher complex with 15 min pre-hybridization was loaded on each HMN followed by drying at 45 degrees for 30 min. This follows by adding another layer of MeHA on top to form the base of the patch. Consecutive adding of MeHA solution and aptamer-quencher complex solution rather than adding a mixture of MeHA and aptamer solution at once reduces the background signal since the aptamer-quencher complex is added only to the needle area, i.e., the base of the patch does not fluoresce. Dry HMN patches were then crosslinked by UV light with 360 nm wavelength for 15 min. The RMFID patches were washed twice with 10 μM of glucose, ATP, L-tyrosinamide or thrombin aptamer binding buffer and further dried under 45 degrees. Last, MN patches were carefully separated from PDMS molds and further crosslinked for 5 mins. After being trimmed and taped on a clean glass slide, the RMFID patches were observed under a fluorescence microscope (Nikon, Ti2). The fabricated needles of the RMFID patch were 850 μm in height, 250 μm in base width and 500 in internal spacing.

In Vitro and Ex Vivo Characterization of RFMID

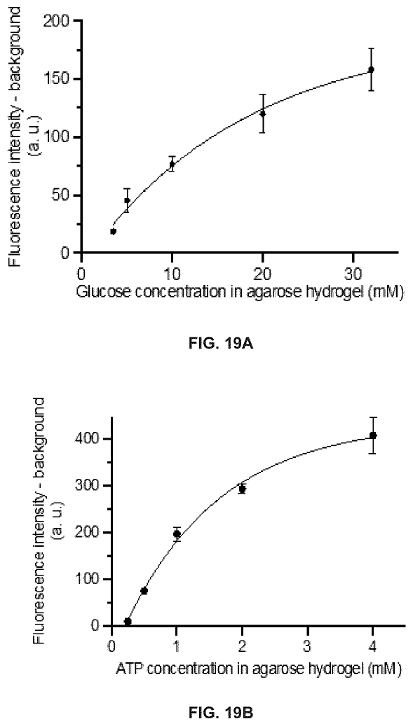

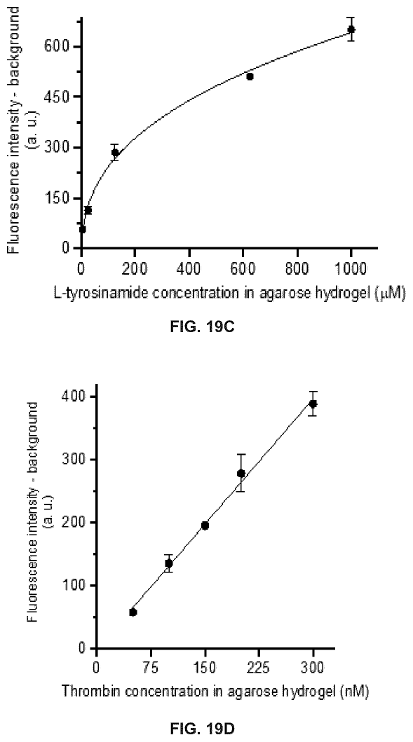

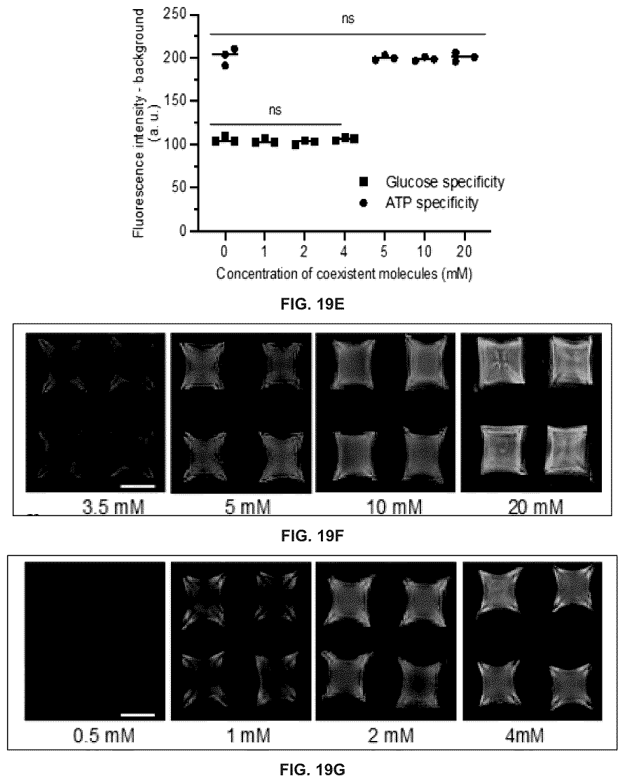

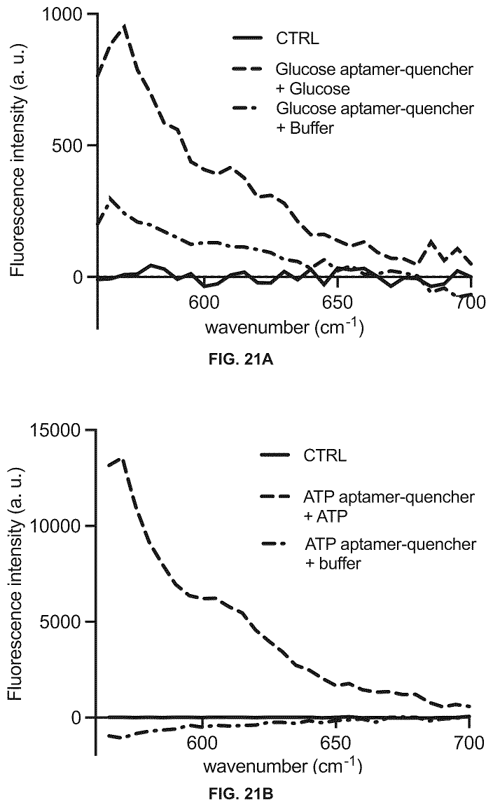

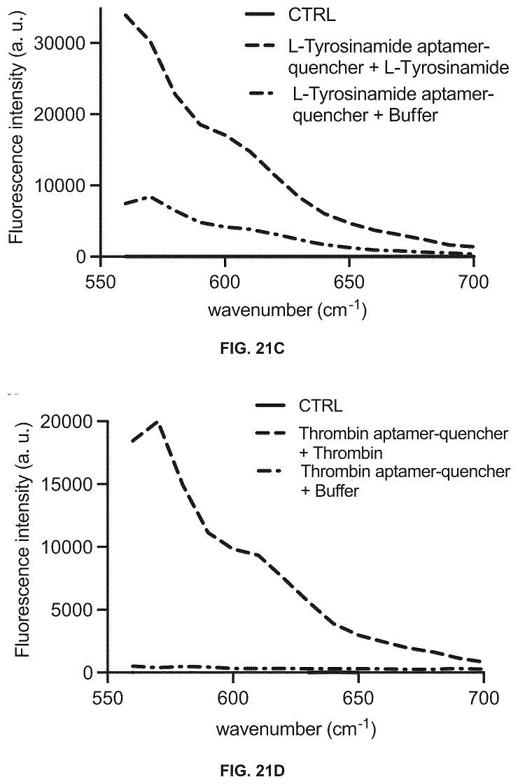

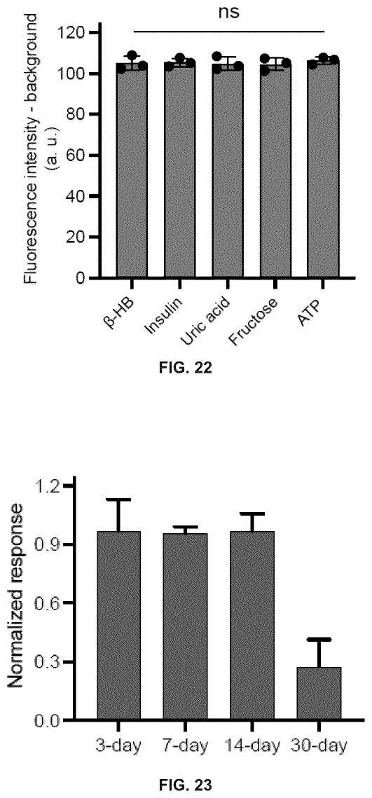

To investigate the sensor's capability for biomolecule detection, a series of experiments were conducted to measure varying concentrations of glucose (FIG. 19A), ATP (FIG. 19B), L-tyrosinamide (FIG. 19C), and thrombin (FIG. 19D) in vitro using HMNs linked with aptamer-quencher strand complex. Sequence and modification of all aptamer and competitor strands are indicated in Table 3. For glucose^{39,40}, ATP⁴¹, and L-tyrosinamide measurement⁴², the specific aptamer probes were hybridized to a quencher strand while for thrombin measurement, the aptamer probe was linked to the quencher strand via a polyethylene glycol (PEG) linker (purchased aptamer probe linked to the quencher strand via PEG)⁴³, averting the release of the quencher strand upon target binding. First studied was the capability of the aptamer-quencher strand complex for target detection, and optimization of the ratio of aptamer to quencher strand (FIGS. 21A-21D). HMN patches linked with suitable ratio of aptamer and quencher strand were then applied on agarose hydrogels loaded with different physiologically relevant concentrations of the target analytes for 10 min Patch fluorescence intensity was measured before and after application to agarose hydrogel and observed that by increasing the target concentration, the signal intensity increases, demonstrating the target capture and dissociation of the quencher strand. To further explore the sensitivity and reliability of detection, cross-reactivity among glucose and ATP was examined by applying HMN patches functionalized with glucose (ATP) aptamer into an agarose hydrogel loaded with a definite concentration of glucose (ATP) while the concentrations of ATP (glucose) increased. It was observed that for both glucose and ATP, the changes of the fluorescence intensity for the non-specific target were almost unnoticeable, indicating the high selectivity of the sensor (FIG. 19E). The cross-reactivity of RFMID device for glucose detection was also studied against fructose, uric acid, insulin, 3-β-Hydroxybutyrate- the dominant biomarker of ketone formation⁴⁴ (FIG. 22). It was observed that the addition of common interfering agents (with a concentration higher than their physiological levels) did not affect the RFMID measurement which demonstrated excellent selectivity of the sensor. FIG. 19F and 19G show that the fluorescence intensity of MN patches increased with the rising glucose and ATP concentration, respectively, confirming elevated dissociation of quencher strand and target capture. Also studied was the storage stability of glucose-RFMID patches and it was shown that the patches can be stored for 14 days at the room temperature with negligible effect on signal measurement (FIG. 23).

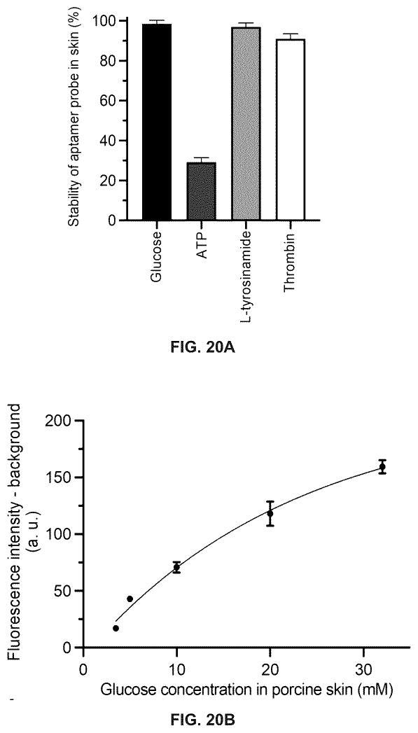

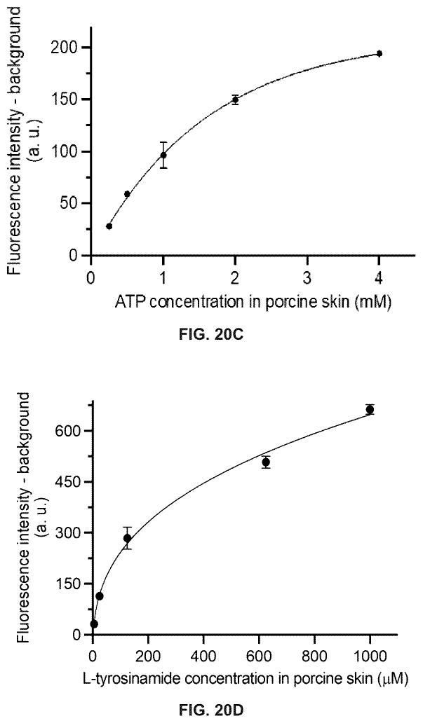

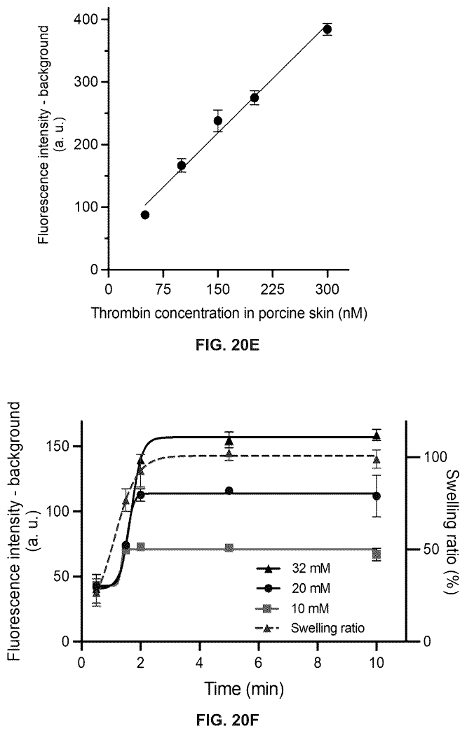

Upon successful detection of target analytes in agarose hydrogel, the sensor capability for in situ biomarker measurement was tested using an ex vivo skin model. First, the stability of aptamer probes in the skin environment against nucleases present in the skin was examined. To this end, functionalized HMN patches with only aptamer probes were applied through a blank porcine skin or agarose hydrogel (with no nucleases present). It was observed that the fluorescence intensity of the patches did not change after insertion. After penetration, the quencher strand was added to the HMN patches and a reduction in fluorescence signal was observed. The reduction of fluorescence signal intensity of the patches applied to skin or agarose were then measured and compared. The fluorescence signal reduction in HMNs penetrated through skin was 98%, 29%, 97%, and 91% of the ones inserted into blank agarose hydrogel for glucose, ATP, L-tyrosinamide, and thrombin aptamer probes, respectively, (FIG. 20A) indicating that most of ATP aptamers became degraded in the skin and could not be hybridized to the corresponded quencher strands. These results suggested that the glucose, L-tyrosinamide, and thrombin aptamers may have good stability in skin, and the ATP aptamer may be relatively less stable in a complex ISF environment.

The RFMID was then employed for glucose (FIG. 20B), ATP (FIG. 20C), L-tyrosinamide (FIG. 20D), and thrombin (FIG. 20E) detection using porcine skin equilibrated with different concentrations of the target analytes. Microneedle traces were evident in the porcine skin, showing an efficient skin penetration. These data were used to construct standard curves that correlated fluorescence signal intensity to target concentration in skin ISF. It was estimated that the limit of detection (LOD) of the biosensor was about three times the standard deviation of the fluorescence signal intensity from a blank agarose hydrogel. The device achieved a LOD of 1.1 mM for glucose, 0.1 mM for ATP, 3.5 μM for L-tyrosinamide and 25 nM for thrombin measurements in skin ISF. The glucose detection limit and dynamic range cover the clinical hypoglycemia, euglycemia and hyperglycemic ranges. The system may be employed to reliably quantify pre- and post-prandial glucose concentrations in patients with diabetes while the accuracy of most of commercially available glucose monitoring devices is still the lowest within the hypoglycemic range⁴⁵.

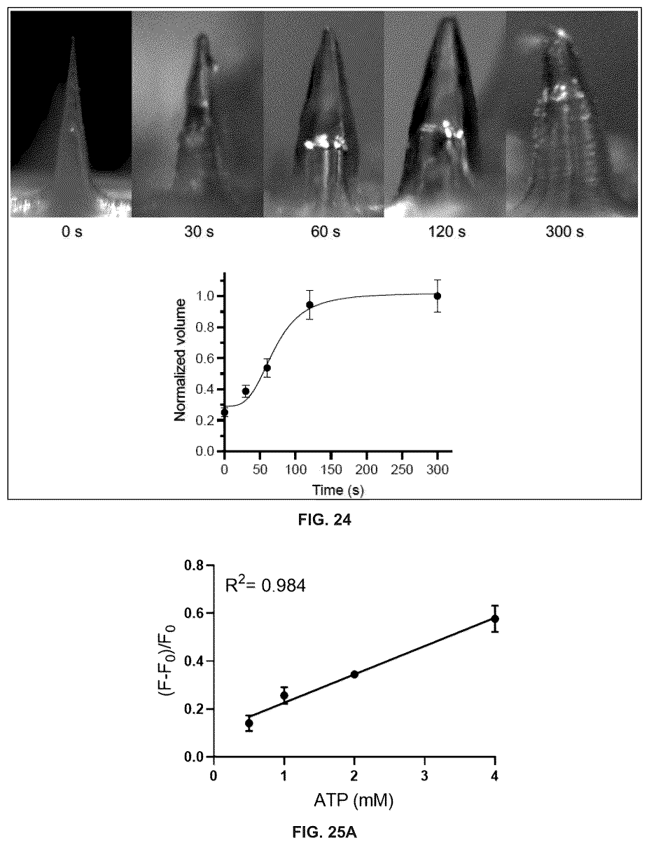

To determine the shortest timescale for effective capture of target analytes, glucose-RFMID patches were applied on porcine skin equilibrated with various concentrations of glucose for different durations. 2 min of microneedle patch administration was found to be sufficient to capture and detect glucose (FIG. 20F) and longer administration (5 and 10 min) did not change the measured fluorescence. The fast response was considered to be because of the swelling characteristic of MeHA-HMNs, which can reach their maximum swelling within 2 min, enabling increased and rapid ISF extraction and thus target capture. The 2 min response time was also independent of the target concentration and increased glucose concentration did not appear to affect the time needed for the target molecules to diffuse into the patch needles. Previously reported MN-based diagnostic devices required at least 140 min to detect the target analytes (Table S1), including the time needed for MN application, washing, and adding the detection agents^{46,47,48}. The change in the volume of needles after insertion was also tracked under microscope (FIG. 24). It was observed that the volume of the needles increased and reached a maximum upon a 2 min insertion, suggesting that a 2 min insertion facilitates reaching the maximum swelling. The results of in vitro and ex vivo characterizations corroborated that the system may be applied for rapid measurement of a diverse range of biomarkers, proteins or small molecules, introducing a generalizable platform for biomolecule quantification.

TABLE 3
Sequence and modification of aptamer and competitor strands

| Name | 5′ mod | Internal mod | 3′ mod | Sequence (5′ to 3′) |
|---|---|---|---|---|
| Glucose aptamer | 5Acryd | iCy3 | | CTCTCGGGACGACCGTGTGTGTTGCTCTGTAACAGTGT |
| | | | | CCATTGTCGTCCC (SEQ ID NO. 13) |
| Glucose competitor | | | 3Dab | GGTCGTCCCGAGAG (SEQ ID NO. 14) |
| ATP aptamer | 5Acryd | iCy3 | | CACCTGGGGGAGTATTGCGGAGGAAGG |
| | | | | (SEQ ID NO. 15) |
| ATP competitor | | | 3Dab | TTTTCCAGGTG (SEQ ID NO. 16) |
| L-tyrosinamide | 5Acryd | iCy3 | | GGAGCTTGGATTGATGTGGTGTGTGAGTGCGGTGCCC; |
| aptamer | | | | SEQ ID NO. 17; |
| | | | | (Acrydite-Cy3-GGAGCTTGGATTGATGTGGTGTGT |
| | | | | GAGTGCGGTGCCC; SEQ ID NO. 17) |
| L-tyrosinamide | | | 3Dab | TCACATCAAT; SEQ ID NO. 18 |
| competitor | | | | (TCACATCAAT-Dabcyl; SEQ ID NO. 18) |
| Thrombin | 5Acryd | iCy3 | 3Dab | CCAAC(CH₂CH₂O)₃₀GGTTGGTGTGGTTGG; |
| aptamer- | | isp18 | | SEQ ID NO. 19 |
| competitor | | | | (Acrydite-Cy3-CCAAC-(CH₂CH₂O)₃₀-GGTTGGT |
| complex | | | | GTGGTTGG-Dabcyl; SEQ ID NO. 19) |
| Thrombin | 5Acryd | iCy3 | | GGTTGGTGTGGTTGG; SEQ ID NO. 20 |
| aptamer | | | | (Acrydite-Cy3-GGTTGGTGTGGTTGG; |
| | | | | SEQ ID NO. 20) |
| Thrombin | | | 3Dab | TTTTTCAACC; SEQ ID NO. 21 |
| competitor | | | | (TTTTTCAACC-Dabcyl-SEQ ID NO. 21) |

Acryd-acrydite modification
Cy3-Cyanine 3 modification
Dab-Dabcyl modification
Isp 18-internal Spacer 18 modification

TABLE S1
Comparison of RFMID's response time with similar MN-based sensors

| | Capture | Washing | Blocking | Detection | Washing | Total time |
|---|---|---|---|---|---|---|
| FLISA¹ | 30 s-20 min | 10 min | 30 min | 120 min incubation with | 10 min | 200 min-220 min |
| | | | | Ab + 30 min incubation | | |
| | | | | with plasmonic fluor | | |
| Aptamer | 60 min | 10 min | — | 60 min incubation with | 10 min | 140 min |
| decorated MN | | | | FAM labeled aptamer | | |
| array² | | | | | | |
| Encoded | 40 min | 10 min | — | 120 min incubation with | 10 min | 180 min |
| MNs³ | | | | detection Ab | | |
| RFMID | 2 min | — | — | — | — | 2 min |

FIGS. 19A-19G shows In vitro characterization of RFMID using an agarose hydrogel model. The RFMID devices functionalized with glucose (a), ATP (b), L-tyrosinamide (c), or thrombin (d) aptamer-quencher complex were applied into agarose hydrogels containing varying concentrations of glucose (3.5, 5, 10, 20, 32 mM), ATP (0.25, 0.5, 1, 2, 4 mM), L-tyrosinamide (5, 25, 125, 625, 1000 μM), or thrombin (50, 100, 150, 200, 300 nM) for 10 min. The fluorescence signal intensity was measured before and after applying the patches and the difference in signal intensity was reported. e, The specificity of RFMID devices for capturing the specific target was tested. The ATP-RFMID devices were applied into agarose hydrogels containing 1 mM of ATP while glucose concentrations increased (0, 5, 10, 20 mM). Similarly, the glucose-RFMID devices were applied into agarose hydrogel containing 20 mM of glucose while ATP concentrations increased (0, 1, 2, 4 mM). f, Fluorescence microscopic images of glucose-RFMID and g, ATP-RFMID after capturing varying concentrations of ATP (0.5, 1, 2, 4 mM) or glucose (3.5, 5, 10, 20, 32 mM) loaded in the agarose hydrogel. Each experiment was repeated three times. Data are presented as mean±s.d. a.u., arbitrary units.

FIGS. 20A-20F shows ex vivo characterization of RFMID using a porcine skin model. a, Stability of the aptamer probes in the skin. RFMID patches functionalized with only aptamer probes were applied to porcine skin or a blank agarose hydrogel. Upon insertion, the corresponding quencher strands were added to the patches. The reduction of fluorescence signal intensity of the patches applied to skin or agarose were then measured and compared. The graph shows 1−[(fa−fs)/fa] as a measure of aptamer stability, where fa and fs are the reduction in fluorescence signal for HMN patches applied into the agarose hydrogel or skin, respectively, upon addition of quencher strands. The RFMID devices functionalized with glucose (b), ATP (c), L-tyrosinamide (d), or thrombin (e) aptamer-quencher complex were applied into porcine skin equilibrated with varying concentration of target analytes for 5 min. The fluorescence signal intensity was measured before and after applying the patches and the difference in signal intensity was reported. Since some ATP aptamer probes were degraded in skin (based on experiments in a), the level of fluorescence signal is less compared with agarose hydrogel experiment in FIG. 19B. f, Glucose-RFMID devices were applied through porcine skin equilibrated with different glucose concentrations (10, 20, and 32 mM) for different durations. The swelling of patches and the fluorescence response were then measured. 2 min was found to be sufficient time of HMN application. Each experiment was repeated three times. Data are presented as mean±s.d. a.u., arbitrary units.

FIGS. 21A-21D shows an assessment of aptamer binding efficiency. The target binding efficiency of a) glucose, b) ATP, c) L-Tyrosinamide, and d) thrombin aptamer was tested with spectrophotometer. A spectral scanning measurement was conducted with the excitation of 530 nm, and an emission from 550 nm to 700 nm. Buffer solution was used as background control (CTRL). The aptamer probe was hybridized to its corresponding quencher for 30 min before adding the targets. Subsequently, the fluorescence intensity difference was measured after target capture for 30 min.

FIG. 22 shows the cross-reactivity of RFMID device for glucose capture in the presence of common interfering agents. In this experiment, agarose hydrogel was loaded with a glucose concentration of 20 mM and β-HB concentration of 10 mM or insulin concentration of 10 nM or fructose concentration of 0.5 mM or uric acid concentration of 0.5 mM or ATP concentration of 4 mM. Data is expressed as mean ±s.d. n=3 replications per group (ns, not significant).

FIG. 23 shows stability test of RFMID patch for glucose measurement. Glucose-RFMID patches were stored at the room temperature for 3, 7, 14 or 30 days and then were applied to agarose hydrogel loaded with 20 mM glucose concentration. The responses were normalized to the measurements extracted from a fresh RFMID patch.

FIG. 24 shows HMN patches applied on agarose hydrogel for different durations (0, 30 s, 60 s, 90 s, 120 s, and 300 s) and the change in the needle volume was observed under microscope.

1.6 Background, Example 1 References
2. Samant, P. P. et al. Sampling interstitial fluid from human skin using a microneedle patch. Sci. Transl. Med. 12, 571-606 (2020).
4. Chang, H. et al. A Swellable Microneedle Patch to Rapidly Extract Skin Interstitial Fluid for Timely Metabolic Analysis. Adv. Mater. 29, 83-122 (2017).
16. Zheng, M. et al. Osmosis-Powered Hydrogel Microneedles for Microliters of Skin Interstitial Fluid Extraction within Minutes. Adv. Healthc. Mater. 9, 1-11 (2020).
18. Zhu, J. et al. Gelatin Methacryloyl Microneedle Patches for Minimally Invasive Extraction of Skin Interstitial Fluid. Small 16, 1-9 (2020).
20. Wang, Z. et al. Microneedle patch for the ultrasensitive quantification of protein biomarkers in interstitial fluid. Nat. Biomed. Eng. 5, 64-76 (2021).
21. Poldervaart, M. T. et al. 3D bioprinting of methacrylated hyaluronic acid (MeHA) hydrogel with intrinsic osteogenicity. PLoS One 12, 1-15 (2017).
22. Bencherif, S. A. et al. Influence of the degree of methacrylation on hyaluronic acid hydrogels properties. Biomaterials 29, 1739-1749 (2008).
26. Il'icheva, I. A., Nechipurenko, D. Y. & Grokhovsky, S. L. Ultrasonic cleavage of nicked DNA. J. Biomol. Struct. Dyn. 27, 391-398 (2009).
27. Nakatsuka, N. et al. Aptamer-field-effect transistors overcome Debye length limitations for small-molecule sensing. Science. 362,319-324 (2019).
28. Poudineh, M. et al. A fluorescence sandwich immunoassay for the real-time continuous detection of glucose and insulin in live animals. Nat. Biomed. Eng. 5,53-63 (2021).
29. Wilson, B. D., Hariri, A. A., Thompson, I. A. P. & Eisenstein, M. Independent Control of the Thermodynamic and Kinetic Properties of Aptamer Switches. Nat. Commun. 10, 1-22 (2019).
31. Boyne, M. S. et al. Timing of Changes in Interstitial and Venous Blood Glucose Measured with a Continuous Subcutaneous Glucose Sensor. Diabetes 52,2790-2794 (2003).
35. GhavamiNejad, A. et al. Glucose-Responsive Composite Microneedle Patch for Hypoglycemia-Triggered Delivery of Native Glucagon. Adv. Mater. 31,1-7 (2019).
37. Yi, K. et al. Aptamer-decorated porous microneedles arrays for extraction and detection of skin interstitial fluid biomarkers. Biosens. Bioelectron. 190, (2021).
38. Zhang, X., Chen, G., Bian, F., Cai, L. & Zhao, Y. Encoded Microneedle Arrays for Detection of Skin Interstitial Fluid Biomarkers. Adv. Mater. 31, 1-8 (2019).
39. Nakatsuka, N.; Yang, K.; Abendroth, J. M.; Cheung, K.; Yang, H.; Zhao, C.; Zhu, B.; Rim, Y. S.; Yang, Y.; Weiss, P. S.; Andrews, A. M.; Angeles, L.; Angeles, L.; States, U.; Angeles, L.; Angeles, L.; States, U.; Approaches, T.; States, U.; Science, B.; Behavior, H.; Angeles, L.; States, U.; Angeles, L.; States, U.; States, U. Aptamer-Field-Effect Transistors Overcome Debye Length Limitations for Small-Molecule Sensing. Science (80-.). 2019, 362 (504901225), 319-324. https://doi.org/10.1126/science.aao6750.Aptamer-field-effect.
40. Poudineh, M.; Maikawa, C. L.; Ma, E. Y.; Pan, J.; Mamerow, D.; Hang, Y.; Baker, S. W.; Beirami, A.; Yoshikawa, A.; Eisenstein, M.; Kim, S.; Vuĉković, J.; Appel, E. A.; Soh, H. T. A Fluorescence Sandwich Immunoassay for the Real-Time Continuous Detection of Glucose and Insulin in Live Animals. Nat. Biomed. Eng. 2021, 5 (1), 53-63. https://doi.org/10.1038/s41551-020-00661-1.
41. Wilson, B. D.; Hariri, A. A.; Thompson, I. A. P.; Eisenstein, M.; Soh, H. T. Independent Control of the Thermodynamic and Kinetic Properties of Aptamer Switches. Nat. Commun. 2019, 10 (1), 1-9. https://doi.org/10.1038/s41467-019-13137-x.
42. Feagin, T. A.; Olsen, D. P. V.; Headman, Z. C.; Heemstra, J. M. High-Throughput Enantiopurity Analysis Using Enantiomeric DNA-Based Sensors. J. Am. Chem. Soc. 2015, 137 (12), 4198-4206. https://doi.org/10.1021/jacs.5b00923.
43. Tang, Z.; Mallikaratchy, P.; Yang, R.; Kim, Y.; Zhu, Z.; Wang, H.; Tan, W. Aptamer Switch Probe Based on Intramolecular Displacement. J. Am. Chem. Soc. 2008, 130 (34), 11268-11269. https://doi.org/10.1021/ja804119s.
44. Laffel, L. Ketone Bodies: A Review of Physiology, Pathophysiology and Application of Monitoring to Diabetes. Diabetes. Metab. Res. Rev. 1999, 15 (6), 412-426. https://doi.org/10.1002/(SICI)1520-7560(199911/12)15:6<412::AID-DMRR72<3.0.CO;2-8.
45. Dungan, K.; Verma, N. Monitoring Technologies—Continuous Glucose Monitoring, Mobile Technology, Biomarkers of Glycemic Control. Endotext. 2000.
46. Wang, Z.; Luan, J.; Seth, A.; Liu, L.; You, M.; Gupta, P.; Rathi, P.; Wang, Y.; Cao, S.; Jiang, Q.; Zhang, X.; Gupta, R.; Zhou, Q.; Morrissey, J. J.; Scheller, E. L.; Rudra, J. S.; Singamaneni, S. Microneedle Patch for the Ultrasensitive Quantification of Protein Biomarkers in Interstitial Fluid. Nat. Biomed. Eng. 2021, 5 (1), 64-76. https://doi.org/10.1038/s41551-020-00672-y. Also Reference (1) in Table 51.
47. Yi, K.; Wang, Y.; Shi, K.; Chi, J.; Lyu, J.; Zhao, Y. Aptamer-Decorated Porous Microneedles Arrays for Extraction and Detection of Skin Interstitial Fluid Biomarkers. Biosens. Bioelectron. 2021, 190 (March). https://doi.org/10.1016/j.bios.2021.113404. Also Reference (2) in Table S1.
48. Zhang, X.; Chen, G.; Bian, F.; Cai, L.; Zhao, Y. Encoded Microneedle Arrays for Detection of Skin Interstitial Fluid Biomarkers. Adv. Mater. 2019, 31 (37), 1-8. https://doi.org/10.1002/adma.201902825. Also Reference (3) in Table 51.

### Example 2

### A hydrogel Microneedle Assay Combined with Nucleic Acid Probes for On-Site Detection of Small Molecules, Proteins, and Ribonucleic Acids

A Versatile Microneedle Biosensor.

Point of care testing (POCT) of clinical biomarkers is important to health monitoring and timely treatment, yet biosensing assays capable of detecting biomarkers without the need for costly external equipment and reagents are limited. Blood-based assays are, specifically, challenging as blood collection can be invasive while pre-processing is required. Herein described is a versatile assay that employs hydrogel microneedles (HMNs) to extract interstitial fluid (ISF), the fluid underneath of skin, in a minimally invasive manner and graphene oxide-nucleic acid (GO.NA)-based fluorescence biosensor to sense the biomarkers of interest in situ. The HMN-GO.NA assay may be supplemented with a portable detector, enabling a complete POCT procedure. The herein described system may successfully measure six clinically important biomarkers (glucose, uric acid, insulin, and serotonin as well as microribonucleic acid 210 and 21) ex-vivo, in addition to accurately detecting glucose and uric acid in-vivo.

Herein described is a sample-in answer-out biosensing platform for detection of various biomarkers applicable to point-of-care-testing.

Introduction

Point-of-care testing (POCT) is generally conducted close to the site of patient care and enables rapid turn-around of results, fast clinical actions, and patient-centered care. POCT is growing rapidly, and its global market could be worth $68.6 billion USD by 2030. To date, blood has been used as the main source of biomarkers; however, blood processing can be labor-intensive, time-consuming, and requires sophisticated clinical equipment, making its use for POCT less favorable. Blood sampling can also be painful and can be challenging for some patients (children, critically ill, and elderly). A promising alternative to blood is interstitial fluid (ISF) which is originated from blood and fills the extracellular space; thus, it has common biomarkers with plasma/serum while also containing biomarkers unique to the local cells. A wide range of metabolites, including amino acids, lipids, and nucleotides as well as protein biomarkers can be detected in ISF, emphasizing the potential of ISF for health monitoring 9.

Microneedle (MN)-based biosensors have emerged as a promising approach for ISF monitoring. They enable minimally invasive penetration through the skin for ISF access and sensing. Off-MN and on-MN biosensors have been reported for analysis of biomarkers of clinical relevance. In the off-MN format, the collected ISF is recovered and used for subsequent analysis. The on-MN refers to the assays in which the recognition elements are integrated, thus enabling on-needle analysis. On-MN assays potentially eliminate the need for sample processing and can be employed for POCT. For instance, solid MNs coated with specific enzymes have been developed to electrochemically detect glucose and ketone bodies—the cause of diabetic ketoacidosis. A flexible MN coated with enzymes has been also reported to simultaneously detect glucose, uric acid, and cholesterol, but has not been tested for in-vivo application¹⁸. Solid MNs integrated with miniaturized electronics have been used to enzymatically detect glucose, alcohol, and lactate in human subjects. Despite these significant achievements, enzyme-based biomarker detection is limited to a narrow range of biomarkers for which an enzyme is available. Non-enzymatic MNs immobilized with antibodies or aptamers have also been reported, but their detection strategy relies on the addition of extra reagents, complicating their POCT application.

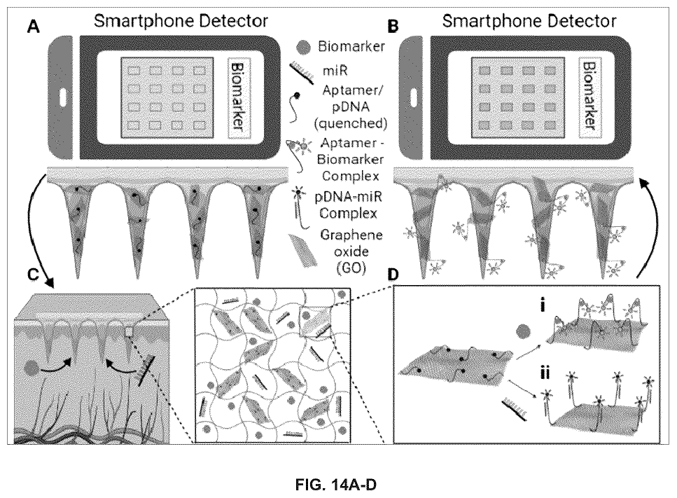

Herein described is an HMN assay that incorporates graphene oxide-nucleic acid (GO.NA) optical sensors for POCT and sensing. The GO.NA optical sensor consists of GO nanosheets conjugated to fluorophore-modified nucleic acid (NA), in which GO acts as a quencher. Single-stranded NA has high affinity for binding to GO, therefore, in the absence of biomarker of interest the NAs are tightly bound to GO; in turn, their fluorophore tag is quenched. However, in the presence of a specific biomarker, the NAs bind to their target, inducing a conformational change that distances the fluorophore tag from GO, leading to fluorescence recovery and generation of an optical signal. GO also acts as a substrate for NA immobilization; therefore, protects the NA from degradation and inhibits potential NA release from HMN-GO.NA. Further, GO quenches the fluorophores, eliminating the need for a quencher-modified displacement strand. To enable microscope-free patch visualization and optical measurements for POCT, further developed was a miniaturized smartphone-based system that captures fluorescence images of the HMN-GO.NA patches (A and B of FIG. 14A-D), which are then analyzed using a freely available software.

FIG. 14A-D provide an overview of the HMN-GO.NA sensing strategy and workflow of the HMN-GO.NA assay, where: (A) to set the baseline fluorescence level (F₀), the HMN-GO.NA patches were first imaged with a smartphone-based detector before skin application, and (C-D) subsequently after skin application when the level of fluorescence increased (B). The response was then defined as the (F−F₀)/F₀. The HMN-GO.NA patches were applied on the skin and penetrated the epidermis where they extract the ISF along with the presented biomarkers (of FIG. 14A-D). The inset illustrates the network of the HMNs after ISF extraction, which is composed of the MeHA hydrogel, GO.NA (the sensing component), and the extracted biomarkers. of FIG. 14A-D provides a schematic illustrating the sensing mechanism of GO.NA; where the NA component is a (i) fluorophore-conjugated aptamer or (ii) fluorophore-conjugated probe DNA (pDNA). In the absence of biomarker/micro ribonucleic acids (miR), the NA remains attached to GO; thus, its fluorophore is quenched; however, in the presence of biomarker/pDNA, aptamer-biomarker and pDNA-miR complexes are formed, inducing a conformational change that distances the fluorophore from GO, leading to the subsequent fluorophore light-up.

Versatility of the HMN-GO.NA was investigated by employing two different types of NAs as the recognition element, aptamer and a probe deoxynucleic acid (pDNA), and detecting a range of different analytes including small molecules, proteins, and micro ribonucleic acids (miR). Aptamers were employed to detect glucose, uric acid (UA), serotonin, and insulin while pDNAs were used to detect miR21 and miR210. Table 4 below outlines the nucleic acid sequences used with their modification. The four biomarkers detected were considered to be of clinical importance for health monitoring. Continuous monitoring and POCT of glucose and insulin levels have long been considered important for diabetic management and mitigation of insulin over/under dose. Changes in UA levels are generally recognized as an important diagnosis and prognosis factor in many multifactorial disorders like obesity, metabolic syndrome, and hypertension UA monitoring can be particularly important in cancer patients who undergo chemo and radiotherapy. Serotonin can be important in regulating certain body functions such as mood, sleep, digestion, nausea, wound healing, bone health, and blood clotting. miRes, a class of small non-coding NAes, are generally considered to act as diagnostic and prognostic biomarkers for various cancers, including breast, colon and lung cancer. More specifically, the levels of miR21 and miR 210 are considered to be associated with tumor size, degree of invasion, and cancer stage. Micro ribonucleic acids (also abbreviated as miRNA) may be used also be used for tracking disease treatment effectiveness. For example, the ratio of biomarker miR210 to miR21 may be used as an indicator for drug treatment success. The HMN-GO.NA described herein showed precise analyte detection both in-vitro and ex-vivo. Further, the assay performance was examined for detection of glucose and uric acid in-vivo, in diabetic rat models. The herein described HMN-GO.NA biosensor along with the miniaturized imager provide a practical solution for disease diagnosis and health monitoring in POCT.

Results

HMN-GO.NA Sensing Strategy

To make HMN-GO.NA assay, a mixture of hydrogel and GO.NA was prepared followed by MN fabrication using the micromolding technique. The fabricated HMNs consisted of an array of 10×10 needles with a height of 850 μm and are capable of penetrating the skin, reaching the epidermis, and extracting ISF in a minimally invasive manner. In the GO.NA complex, the NA was conjugated to a fluorophore tag (Cy3) and acted as the biorecognition element; recognizing its specific biomarker and binding to it, while GO acted as the quencher element, quenching the fluorophore tag in the absence of the biomarker. GO was linked to two different types of NAs: aptamers for small molecule, metabolite, as well as protein detection, and probe DNA (pDNA) for miR detection. As illustrated in of FIG. 14A-D, upon HMN-GO.NA insertion into the skin, ISF along with its biomarkers diffused into the swelled needles. In the presence of the specific biomarker, an aptamer-biomarker complex formed, inducing a conformational change that dissociated the fluorophore tag from GO, leading to fluorophore light-up (Di of FIG. 14A-D). Similarly, if miRs of interest existed in the extracted ISF, a pDNA-miR complex would be formed, which distanced the pDNA-conjugated fluorophore from GO, leading to illumination of fluorophore (Dii of FIG. 14A-D). The increase in the fluorescence signal was proportional to the concentration of target analytes, allowing accurate analyte quantification.

HMN-GO.NA Fabrication and Characterization

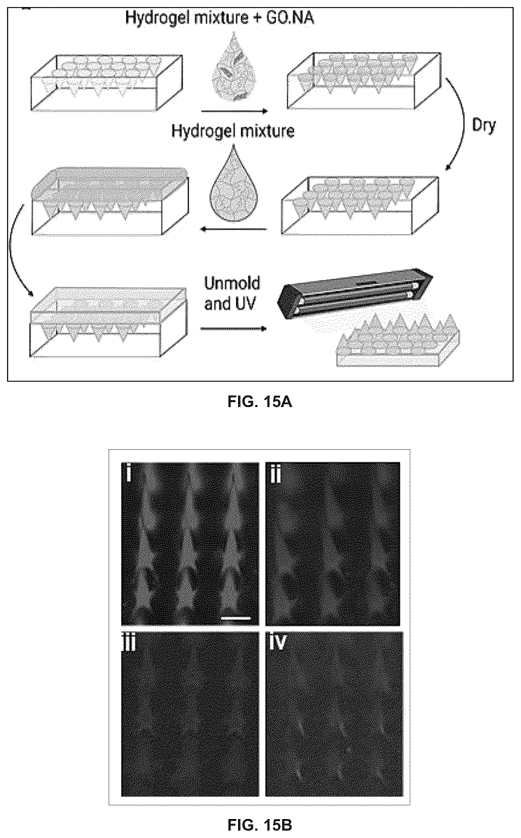

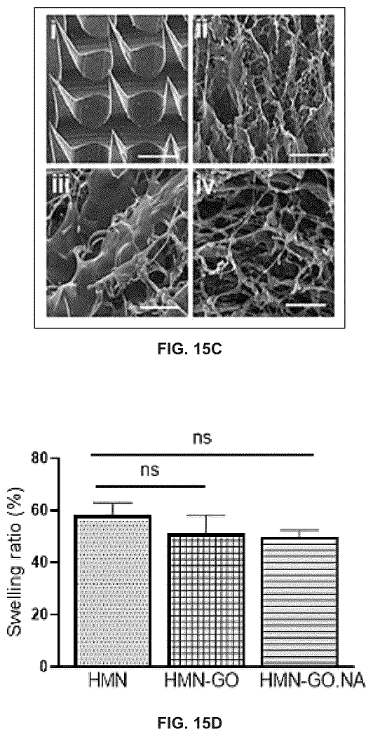

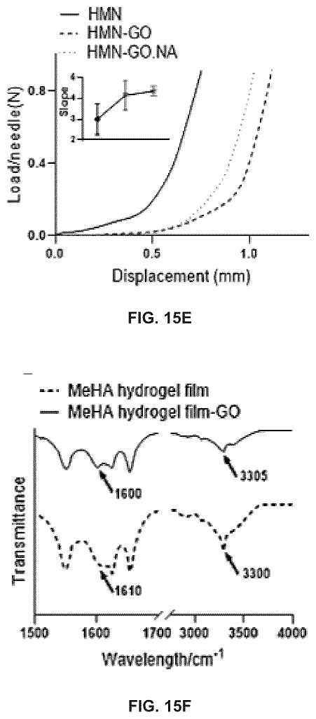

To fabricate the HMN-GO.NA patches, hyaluronic acid (HA), a highly biocompatible polymer, was modified with methacrylic anhydride to form the methacrylated HA (MeHA) as the hydrogel backbone. MeHA has been previously used for the fabrication of HMN arrays and has displayed a high degree of swelling, allowing increased ISF extraction and improved sensing^{21,37}. HMN-GO.NA was then fabricated using a two-layer micro-molding technique. The first layer or the needle region is composed of a mixture of a hydrogel and GO.NA which was injected to the mold (FIG. 15A). Photoinitiator (PI) and a crosslinking agent, N′-methylenebisacrylamide abbreviated as MBA, were also added to the hydrogel mixture to make a crosslinked hydrogel network. The second layer or the base is composed of hydrogel mixture which was added after the needle region was semi-dry (FIG. 15A). The two-layer fabrication technique prevents possible base deformation and enabled the formation of sharper needle tips. Subsequent to adding the two layers, the HMN-GO.NA patches were dried at room temperature and UV radiated to induce crosslinking.

Investigated were two approaches to make the GO.NA complex: covalent bonding and physical adsorption (physisorption). In the covalent bonding approach, a covalent bond was formed between the carboxyl groups of GO and the amine groups on the amine-modified NA strands, resulting in the covalent complex of GO.NA. In the physisorption approach, pi-pi stacking and hydrophobic interactions between the NA rings and the hexagonally shaped carbons in GO produced the strongly conjugated GO.NA complex. To assess the fluorescence quenching capability of GO, HMN-GO.NA was fabricated where NA was linked to GO either covalently or physically, and the HMN patches were imaged using a microscope. In the absence of GO or a quencher-conjugated displacement strand, a strong fluorescence signal was observed (FIG. 15Bi). The fluorescence signal was significantly reduced when a quencher-conjugated displacement strand (e.g., see Example 1) was employed (FIG. 15Bii). HMN patches with covalently—(FIG. 15Biii) or physically—(FIG. 15Biv) linked GO.NA also demonstrated a very low fluorescence signal, highlighting the quenching capability of GO. Knowing that GO could effectively quench the fluorescence, the characteristics of the HMN-GO.NA patches were investigated. Scanning electron microscopy (SEM) was performed to visualize HMN-GO.NA array with pyramid-shaped, sharp needles, which facilitate effective skin penetration (FIG. 15Ci). Next studied was the pore size of hydrogel, hydrogel-GO, and hydrogel-GO.NA films using SEM imaging (FIG. 15C). Hydrogel-GO and hydrogel-GO.NA with an average pore size of 3.9 and 3.6 μm, respectively, showed larger pores than the hydrogel films with no GO addition which had an average pore size of 1.8 μm. This event may be due to repulsion by GO sheets. Next examined was the effect of GO and GO.NA on the swelling ratio of HMN patches. HMN patches without and with GO and GO.NA complexes were fabricated, and their swelling ratios were measured based on the patches' weight before and after 10 minutes of agarose hydrogel application. The addition of GO did not appear to have a significant effect on HMN swelling ratio as the HMN, HMN-GO, and HMN-GO.NA showed close swelling ratios of 58%, 50%, and 51%, respectively (FIG. 15D). To crosslink the HMN-GO.NA patches, a long UV radiation of 40 min was used, which led to a lower swelling ratio (58%). Next, the mechanical strength of the HMN, HMN-GO, and HMN-GO.NA patches were evaluated using a compression test that recorded the applied force versus displacement and showed the load sustained by needles (FIG. 15E). The slope of force-displacement graphs—an indicator of mechanical strength—was calculated enabling further quantification of mechanical strength for each patch. HMN patches incorporated with GO showed slightly higher slopes compared with patches without GO, suggesting that the addition of GO had slightly increased the mechanical strength (FIG. 15C).

Next characterized was the interaction of GO with the MeHA hydrogel network. GO contains carboxyl groups which can act as the hydrogen acceptor and form hydrogen bonds with the amine and/or hydroxyl groups present in the MeHA hydrogel. To show the formation of such hydrogen bonds between GO and MeHA, Fourier Transform Infrared (FTIR) spectroscopy was performed on hydrogel and hydrogel-GO films. The N-H stretching vibration peak at 3300 in the hydrogel-only spectrum shifted to 3305 in the hydrogel-GO spectrum, indicating the formation of hydrogen bonds. The N—H bending peak at 1610 also shifted to 1600 in the hydrogel-GO spectrum, further suggesting hydrogen bond formation between GO and the MeHA hydrogel^{44,46}. Even though GO is highly biocompatible, the formation of hydrogen bonds between GO and MeHA hydrogel ensured effective GO entrapment inside the microneedles and relieved any concern regarding potential GO release. Also studied was the release of NA from HMN-GO.NA patches (FIG. 15G). Fluorophore-conjugated adenosine three phosphates (ATP) aptamer was used as the NA biorecognition element to construct HMN patches incorporated with covalently and physically linked GO.NA complex. A control sample was also fabricated by simply mixing MeHA hydrogel with NA without incorporation of GO (HMN-NA). The fabricated HMN patches were immersed in a buffer solution containing a high concentration of ATP for 5, 10, and 30 minutes, after which the patches were withdrawn, and the buffer was analyzed for the presence of the fluorophore-modified NA. As FIG. 15C shows, the observed fluorescence intensity (FI) for HMN-NA patches has increased by increasing the incubation time, demonstrating the release of NA into buffer solution due to the loose entrapment of NA in the hydrogel matrix. However, such an increase was not observed for the covalent HMN-GO.NA patches (FIG. 15G). The physisorbed HMN-GO.NA patches also showed an increase in Fl as a result of longer incubation time, yet their increase was much lower than that of HMN-NA. These observations suggested that GO plays a role in retaining the NA inside HMN patches; thereby, preventing NA release and maintaining functional biomarker sensing.

FIGS. 15A-15G provided an overview of HMN-GO.NA assay fabrication and characterization. More particularly, FIG. 15 depicts (A) Schematic showing the fabrication process for the two-layered HMN-GO.NA assay; (B) Fluorescence images of the (i) HMN with NA-conjugated with fluorophore, (ii) HMN with aptamer conjugated with fluorophore-displacement strand conjugated with quencher complex, (iii) HMN with covalent GO.NA, and (iv) HMN with physisorbed GO.NA (scale bar=250 μm, and LUT=100-10000, exposure time (EP)=10 ms for all except i with EP=5 ms); (C) SEM images of (i) the HMN-GO.NA array (scale bar=250 μm) as well as (ii) MeHA hydrogel film (scale bar=20 μm), (iii) MeHA hydrogel film with GO (scale bar=12 μm), and (iv) MeHA hydrogel film with GO.NA (scale bar=8 μm) to analyze the pore sizes; (D) The analyzed swelling ratio percentage for HMN, HMN-GO, and HMN-GO.NA showing no significant difference after the addition of GO or GO.NA to the HMN patch (P <0.05), where at least four patches were tested per condition and the error bars show the SEM; (E) The obtained mechanical strength for HMN-only, HMN-GO, and HMN-GO.NA patches with the inset showing the slope of the force-displacement figure for two patches and the error bar represents SEM; (F) The FTIR performed on the MeHA hydrogel film and MeHA hydrogel film with GO; (G) The NA release experiment which assessed the release of fluorophore-tagged NA with and without conjugation to GO, showing the fluorescence intensity observed in the buffer after 5, 10, and 30 minutes of incubating the patches, where—to account for possible changes that hydrogel release might have caused on the fluorescent signal—all the obtained fluorescent signals were normalized to the fluorescent signal of PBS that was incubated with the hydrogel-only patches and each experiment was performed three times and the error bars represent standard error of the mean (SEM).

Ex-Vivo Validation of HMN-GO.NA Assay for Biomarker Detection

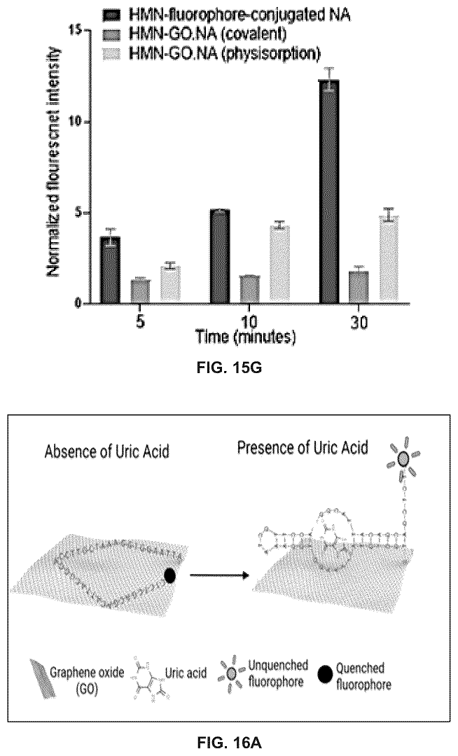

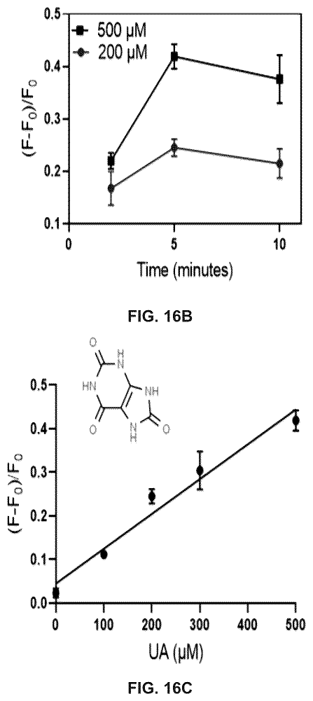

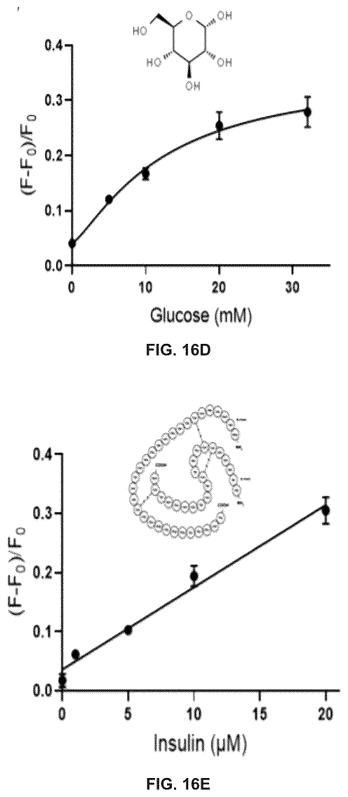

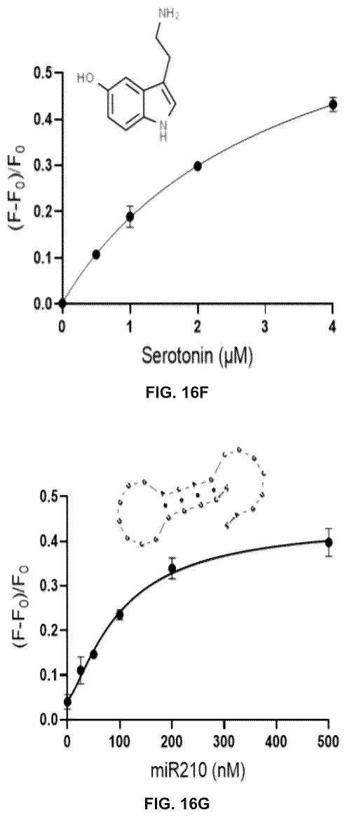

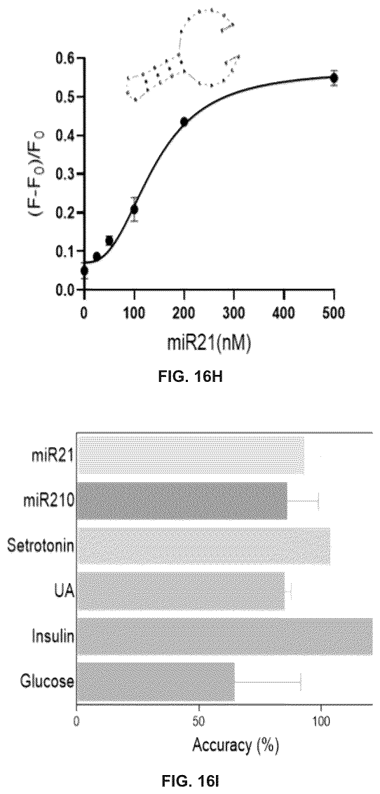

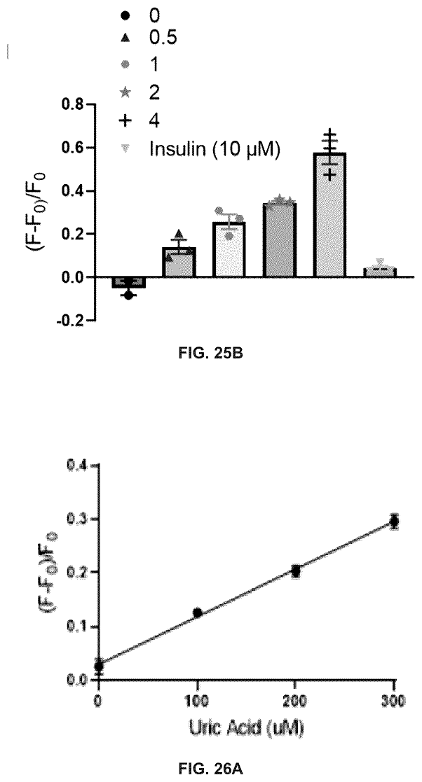

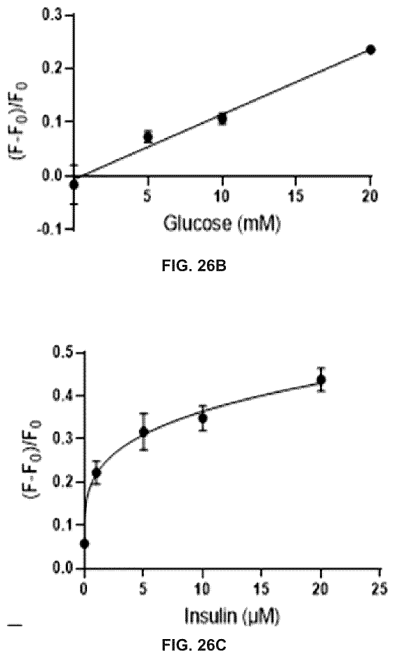

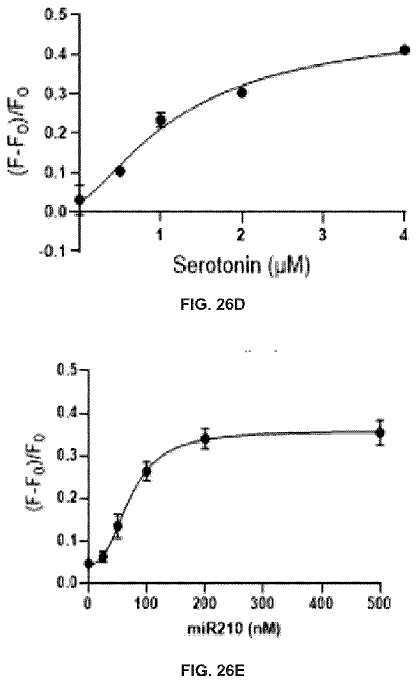

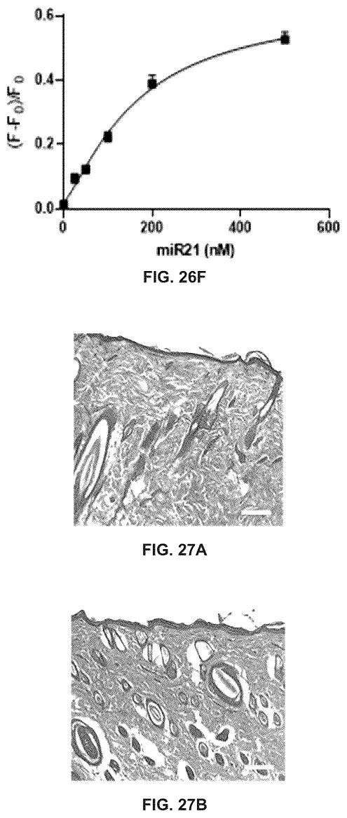

After studying the characteristics of HMN-GO.NA assay, its performance was tested for detection of a diverse range of biomarkers, including small molecules (UA, glucose, and serotonin), proteins (insulin), and ribonucleic acids (miR21 and miR210). The fabricated HMN-GO.NA patches were tested using agarose hydrogel (FIG. 25A, FIG. 25B, and FIGS. 26A-26F) and porcine skin (FIGS. 16A-16) loaded with different concentrations of the targets of interest. Tested HMN-GO.NA assay for detection of UA, high concentration of which is generally associated with the development of gout disease, renal failure, hypertension, hyperlipidemia, diabetes, and obesity FIG. 16A represents the sensing mechanism of the HMN-GO. NA patches using a selected UA aptamer and UA as the target biomarker. In the absence of UA, the UA aptamer was tightly bound to the GO surface; thus, its fluorophore was quenched, but in the presence of UA, UA aptamer bound to UA causing a conformational change that detached the fluorophore from GO, generating a fluorescent signal. To establish the shortest duration for patch insertion, the HMN-GO.UA.aptamer patches were applied on porcine skins containing 200 and 500 μM of UA for 2, 5, and 10 minutes (FIG. 16B). 5 minutes appeared to be a good timespan for HMN-GO.NA action, as the highest response and response-difference (between 200 and 500 μM UA) was observed after 5 minutes (FIG. 16B). For the rest of the ex-vivo analysis, the HMN-GO.NA patches were applied for 5 minutes. The physiological range of UA in healthy adults is generally between 90-360 μM in women and 150-420 μM in men, with concentrations higher than upper limits considered hyperuricemia . The HMN-GO.UA.aptamer patches were able to accurately detect UA in both physiological and hyperuricemia ranges with a limit of detection (LOD) of 32 μM (FIG. 16C). HMN-GO.NA aptamer was also employed for glucose detection. To fabricate the HMN-GO.Glucose.aptamer patches, physiosorbed GO.Glucose.aptamer conjugates were used Fasting glucose level ranges between 3.9 to 10 mM in healthy adults, with glucose levels lower than 3.9 mM and higher than 10 mM being considered hypoglycemia and hyperglycemia, respectively. The HMN assay could accurately detect glucose in its biological and more importantly pathological level with a LOD of 2.1 mM (FIG. 16D).

Next studied was the detection of insulin, a small protein hormone secreted from the pancreas with a physiological range of 0.1-3 nM, using the assay . Insulin regulates glucose levels in the blood and its monitoring is needed for diabetes management 26. The response of the HMN-GO.Insulin.aptamer patches linearly correlated with insulin concentration showing a LOD of 1.3 μM (FIG. 16C). Also employed was the HMN.GO.NA assay for detection of serotonin, a small-molecule neurotransmitter involved in numerous physiological processes in both central and peripheral nervous systems. Serotonin exists in the biological range of 0.5-1.6 μM while higher concentrations require further clinical investigation. Conventional techniques for serotonin detection mainly rely on liquid chromatography-electrochemical detection (HPLC-ECD), which may be cumbersome and require extensive sample pre-treatment. The HMN-GO.serotonin.aptamer could accurately identify serotonin levels from 0.5-4 μM with an LOD of 0.1 μM (FIG. 16F); offering an effective solution for POCT detection of serotonin. Moreover, the obtained LOD using the herein described method was comparable to the LOD of current common-practice methods for serotonin detection.

To detect miR210 and miR21, HMN-GO.pDNA patches were prepared and applied on skins containing different concentrations of the target miR. The observed fluorescent response from HMN-GO-pDNA patches increased as higher concentrations of miR 210 and miR 21 were present (FIG. 16 G and FIG. 16H). An LOD of 49 nM and 32.8 nM was obtained for miR21 and miR210, respectively (FIG. 16G and FIG. 16H).

FIGS. 16A-16I provides an overview of ex-vivo validation of HMN-GO.NA for biomarker detection. Particularly, FIGS. 16A-16I depicts: (A) a schematic illustrating the mechanism through which GO.UA.aptamer detects UA and produces a fluorescence signal; in the absence of UA, the single-stranded UA aptamer is bound to GO, while, in the presence of UA, the aptamer-UA binding induces a conformational change in the UA aptamer that separates the fluorophore from GO, resulting an increase in the fluorescence signal; (B) the HMN-GO.UA.aptamer patches, applied on porcine skins containing 200 and 500 μM UA and the fluorescence response [(F−F₀)/F₀] was measured after 2, 5, and 10 minutes of patch insertion, 5 minutes was selected as the optimum HMN-insertion time; (C) HMN-GO.UA.aptamer, (D) HMN-GO.Glucose.aptamer, (E) HMN-GO.Insulin.aptamer, and (F) HMN-GO.Serotonin.aptamer were applied on porcine skin containing different concentrations of UA, glucose, insulin, and serotonin, respectively; the response [(F−F₀)/F₀ ] was measured and plotted against the biomarker concentration, a strong linear correlation with R² of 0.96 and 0.98 was observed for HMN-GO.NA patches that detected UA and insulin, respectively; HMN-GO.pDNA for detection of (G) miR210, and (H) miR21 were also prepared and applied on porcine skins containing different concentrations of miR210 and miR21, the fluorescence response increased when a higher concentration of miR210 and 21 was present; (1) HMN-GO.NA patches specific to each biomarker were prepared and applied on porcine skins containing all the biomarkers, the obtained response [(F−F₀)/F₀] was interpolated in the established calibration curves and the percentage accuracy was calculated as [1−(calculated concentration of biomarker/actual concentration of biomarker)]×100, with each experiment performed three times and the error bars represent SEM.

Next studied was the selectivity of the HMN assay for the detection of specific biomarkers in the presence of other interfering biomarkers. Porcine skins containing certain concentrations of all the tested biomarkers (UA, glucose, insulin, and serotonin for non-NA targets and miR210, and miR21 for NA targets) were prepared and HMN-GO.NA patches specific to each target were applied on them. The patch response was then compared with the ones calculated from the experiments with no interferences. The percentage accuracies of 64, 123, 85, 103, 86, 93 were acquired for glucose, insulin, UA, serotonin, miR210, and miR21, respectively. Unlike other patches with accuracy percentages near 100%, HMN-GO.glucose.aptamer patch showed 64% accuracy, which may be due to using physisorbed GO.glucose aptamer conjugate. Physisorption is a physical form of linkage and may be more prone to nonspecific detachment.

These results demonstrated that HMN-GO.NA assay can be applied for rapid, accurate, and specific identification of a range of different analytes including small molecules, proteins, and miRs, highlighting the universality of the assay.

A Portable, Smartphone-Based Detector for POCT Setting

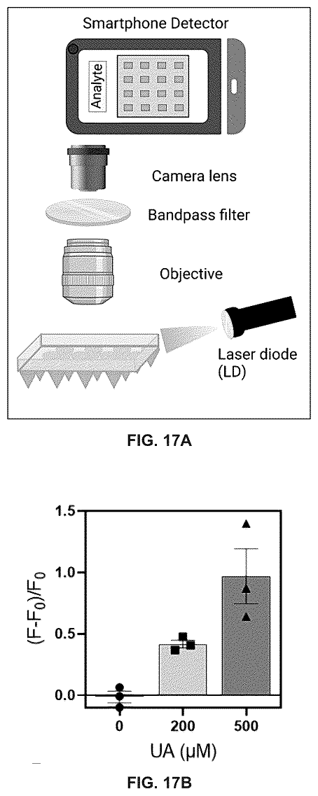

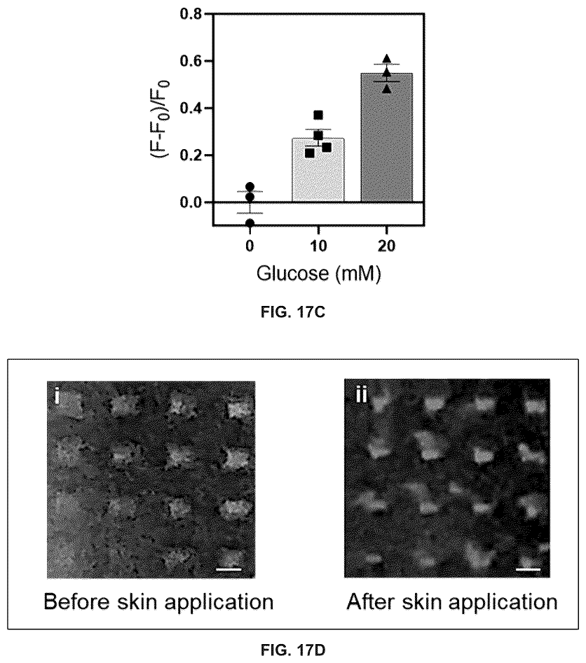

Although fluorescence-based biosensors provide high sensitivity, an obstacle is their need for sophisticated and bulky microscope imagers that can trigger the fluorophores, capture the emitted signal, and visualize the images. To address this challenge and enable the HMN-GO.NA assay for on-site, POCT biomarker detection, a miniaturized optic system and fluorescence imagers was developed, consisting of a laser diode, microscopic objective, and filter as shown in FIG. 17A. The HMN-GO.NA patch was excited by a 532 nm laser diode, corresponding to the excitation of Cy3 fluorophore, and the fluorescence signal was collected by the smartphone detector through a 4× objective lens and bandpass filter (center wavelength of 570 nm and 10 nm full-width half maximum known as FWHM). The filter only allowed the fluorescence signal to pass through and reach the detector of the smartphone and blocks the background light.

HMN-GO.NA patches for UA and glucose detection were prepared, applied on porcine skins containing different concentrations of the targets, and visualized using the developed miniaturized optic system, and the images were analyzed using the freely available software. An increased fluorescence response was observed when higher concentrations of UA and glucose were present in the porcine skin (FIG. 17B-17C), indicating that the developed optic system could effectively capture images and successfully replace previously used fluorescence microscopes. An example of an imaged HMN-GO.UA.aptamer patch before and after introduction to 200 μM UA is presented in FIG. 17D, showing a brighter image after UA capture.

FIGS. 17A-17D provides an overview of a portable and smartphone-based detector for POCT setting. Particularly, FIG. 17A-17D depicts: (A) a schematic showing the components of the developed smartphone-based detector, where the laser diode excites the Cy3 fluorophores at 532 nm, leading to fluorophore emission at 570 nm, the emitted fluorescent light is magnified by the 4× objective, passed through the filter (which filters out background excitations), collected by the camera lens, and visualized by the smartphone (e.g., an iPhone 13); (B)/(C) representative figures showing the fluorescence response [(F−F₀)/F₀] of (B) HMN-GO.UA.aptamer and (C) HMN-GO.glucose.aptamer patches following insertion in porcine skins containing different concentrations of their respective biomarkers, the observed background fluorescence was subtracted from F and F₀, at least three patches were tested per condition and the error bars represent SEM; (D) representative images captured by smartphone-based detector i) before and ii) after applying the HMN-GO.UA.aptamer on porcine skin containing 200 μM UA (scale bar=200 μm).

In-Vivo UA and Glucose Detection in an Animal Model of Diabetes

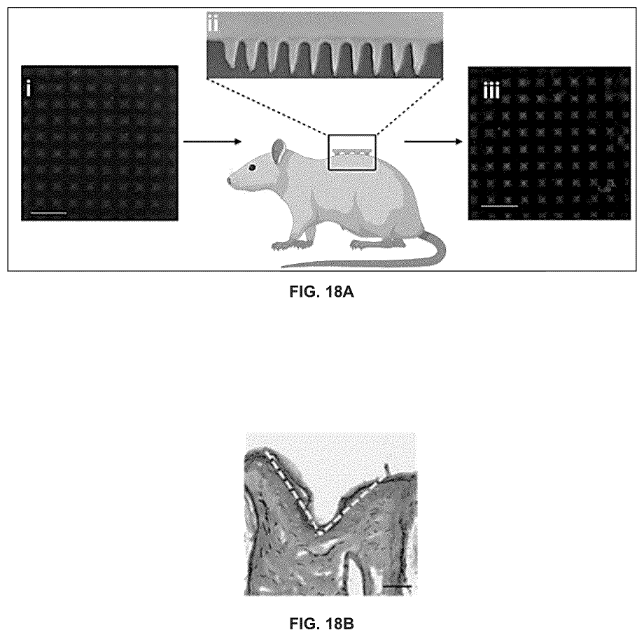

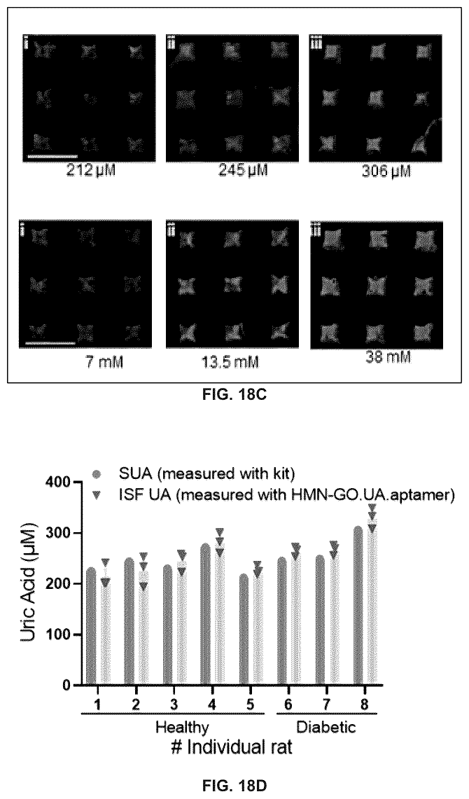

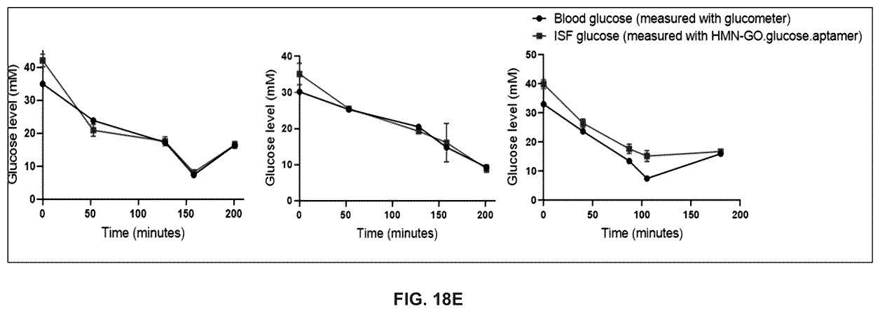

The levels of serum uric acid (SUA) are generally found to be elevated in diabetic patients. The elevated SUA level acts as a key risk factor predicting diabetic-associated complications like stroke, kidney deterioration, and fatty liver disease necessitating SUA monitoring in diabetic patients in addition to glucose. The available at-home UA test kits like HumaSens^{plus} and UASure require fingertip punctures, which can hinderfrequent UA measurement and discomforts patients—specifically children and elderly patients—raising the need for a painless and convenient UA test. Further, the at-home UA test kits like UASure, cannot detect UA concentrations lower than 170 μM limiting their application for diagnosis of certain diseases associated with low UA level^{60,62}. Importantly, 100% of the SUA can also be found in ISF, qualifying a MN-based UA detection method. Thus, employed was the HMN-GO.NA patches to identify levels of UA and glucose in diabetic rats, considering the importance of UA and glucose measurement. To confirm that the patches would not evoke any inflammatory response, hematoxylin and eosin (H&E) staining was performed on the parts of skin that were inserted with HMN patches (FIG. 27A, and FIG. 27B). The control skin with no patch application and the skin treated with HMNs did not show significance difference in histological appearance, confirming the safe and inflammation-free nature of HMN-GO.NA. The workflow for HMN-GO.NA application is illustrated in FIG. 18A. HMN-GO.NA patches were imaged prior to insertion; values of which used to establish the baseline signal, then applied on the rat's dorsal skin for 5 minutes, followed by another round of imaging; values of which were used to measure the produced signal (FIG. 18A). Successful penetration of HMN-GO.NA patches were confirmed, by performing H&E staining and visualizing the needle's cavity with an approximate depth of 900 μm (FIG. 18B). Knowing the MN can effectively penetrate, next was to identify the level of UA and glucose presented in ISF (ISF UA and ISF glucose). FIG. 18C represents HMN-GO.NA patches after being applied on rats with low (FIG. 18C, i), medium (FIG. 18C, ii), and high (FIG. 18C, iii) levels of UA (top) or glucose (bottom). FIG. 18D shows the results of UA measurement using HMN-GO.UA.aptamer assay. Concurrent with needle application, blood was collected from rat tail, and SUA levels were analyzed using a benchmark enzymatic test. The observed levels of ISF UA (obtained with HMN-GO.UA aptamer) closely matched the SUA level in individual rats indicating the accuracy of HMN-GO.UA.aptamer assay in detecting UA (FIG. 18D). Furthermore, consistent with previous findings, diabetic rats (with an average UA level of 287 μM) had higher UA than healthy rats (with an average UA level of 228 μM). HMN-GO.glucose.aptamer was used to detect fluctuating levels of glucose in the ISF of diabetic rats (FIG. 18E). To vary the glucose level in blood and ISF, the diabetic rats were first fasted for 5 hours which caused a significant increase in their glucose level, followed by insulin injection which gradually decreased the glucose level, and a subsequent feeding which again increased glucose levels. During these alterations, the glucose levels were analyzed using HMN-GO.glucose.aptamer patches along with a glucometer. The ISF glucose level (detected with HMN-GO.glucose.aptamer) were well-correlated with blood glucose level (measured with glucometer) in all three tested rats (FIG. 18E), further confirming the accuracy of HMN.GO.NA patches to detect biomarkers in live animals.

FIG. 18A-18E provides an overview of in-vivo UA and glucose detection in an animal model of diabetes. Particularly, FIG. 18A-18E depicts: (A) a schematic illustrating the workflow for applying HMN-GO.NA on diabetic rats, pictures representing the (i) fluorescence and (ii) bright field image of an HMN-GO.NA before and (iii) fluorescent image after insertion into the dorsal skin of diabetic rats (LUT=100-400, EP=10 ms); (B) histology image showing a needles cavity (scale bar=500 μm); (C) fluorescence images representing the HMN-GO.UA.aptamer (top, LUT=100 — 500. EP=10ms) and HMN-GO.glucose.aptamer (bottom, LUT=200-1200, EP=10 ms) after insertion on rats with serum UA levels of (i) 212 μM, (ii) 245 μM, and (iii) 306 μM and blood glucose levels of (i) 7 mM, (ii) 13.5 mM, and (iii) 38 mM; (D) figure representing the mean SUA levels measured with an enzymatic kit next to the mean ISF UA levels measured with HMN-GO.UA.aptamer, three patches were tested per rat and error bars represent SEM; (E) figures representing the mean of blood glucose level tested with a glucometer along with the mean of ISF glucose levels measured with HMN-GO.glucose.aptamer patches in three individual diabetic rats, at least three patches were applied per condition and error bars represent the SEM.

Discussion

In the presented work, an GO.NA optical sensor was integrated with HMN arrays; thereby, leveraging from the competitive advantages that HMN offers (such as biocompatibility, simpler fabrication process, and higher ISF extraction) as well as the versatility of the GO.NA sensors. In the GO.NA conjugate, the NA component was tagged with a fluorophore and acted as the recognition element; while the GO component quenched the fluorophore tag when the biomarkers of interest were not present. It was demonstrated that the GO could successfully quench fluorophore tags; thus, providing a versatile quenching mechanism that fits different types of NA probes (including but not limited to single stranded complementary probe DNA, aptamer, peptide nucleic acid, nucleic acid enzyme, or combinations thereof). Incorporating GO in HMN did not impose a significant change on the swelling ratio of HMN patches, but increased the mechanical strength of patches. The GO was entrapped in the hydrogel matrix by forming hydrogen bonds, preventing GO as well as NA releases.

Employed were two different types of single-stranded NAs namely, aptamer and pDNA (single-stranded NA complementary to miR) in the HMN-GO.NA patches to detect a wide range of biomarkers considered to be clinically important ,as well as miRs presented in ISF. HMN-GO.NA assay was tested for six clinically important biomarkers: UA, glucose, serotonin, insulin, miR210, and miR 21. Additionally, the HMN-GO.NA design was compatible with both covalent (all the GO.NAs except GO.glucose.aptamer) and physiosorbed GO.NA (GO-glucose.aptamer) conjugates, further advancing the versatility of the technique. Moreover, a smartphone-based detector was designed and assembled which allows the HMN-GO.NA implementation at POCT setting. The developed HMN-GO.NA assay was also used to identify the ISF UA and ISF glucose levels in diabetic rats, results of which were compared to a benchmark assay and further validated the accurate performance of HMN-GO.NA assay.

The developed HMN-GO.NA assay may be useful for POCT testing and adaptable for the simultaneous detection of multiple biomarkers. For example, simultaneous detection of glucose and insulin, which is needed for high-quality diabetic management. To enable the HMN-GO.insulin.aptamer for detecting insulin at the physiological ranges (0.05-3 nM), the obtained LOD for insulin could be decreased by 1000 fold, which can be achieved by employing strategies like utilizing an insulin aptamer with higher affinity to insulin, amplifying the fluorescence signal by using a stronger fluorophore like quantum dots, and retaining the insulin target in the detection cycle for several rounds by degrading the insulin-bound aptamer. HMN-GO.serotonin.aptamer assay also demonstrated a great capacity for accurately detecting serotonin at biological and pathological ranges. The prototyped smartphone-based detector could effectively detect and visualize fluorescence responses from HMN patches. Use of the smartphone-based detector may be further enabled by preserving consistent parameters for each component by fixing them in a black box, and using an internal image analyzer.

Method

Synthesize of MeHA

MeHA was synthesized based on the modified protocol established above. Briefly, 2.0g HA was dissolved in 100 mL Millipore water and stirred overnight under 4 degrees for complete dissolving. Subsequently, 1.6 mL MA was added to the HA solution followed by stepwise addition of 3.6 mL of 5N NaOH solution was added to adjust the solution to pH 8 9. The mixture was stirred overnight under 4 degrees to complete the reaction. Next, MeHA was precipitated by acetone and washed three times with ethanol. Subsequently, precipitated MeHA was redissolved in Millipore water and was dialyzed for 5 days to remove the impurity. The purified MeHA was lyophilized for 3 days.

Synthesize of GO. NA Conjugates

Amine-modified NAs were covalently linked to GO using a modified protocol from [24]. Briefly, 100 μg/mL GO was mixed with freshly prepared 10 mM N-(3-Dimethylaminopropyl)-N′-ethylcarbodiimide hydrochloride EDC-HCl, 25 mM NaCl, 25 mM 2-(N-morpholino)ethanesulfonic acid MES, and 2 μM amine-modified NA in a medium of water. The mixture was stirred at room temperature for 3 hours, and the GO.NA complex was purified by 20 minutes of centrifugation at 14000 rpm followed by 2 times washing with nuclease-free water. After the last centrifugation, the GO.NA pellet was dispersed in buffer A (100 mM NaCl, 25 mM HEPES, pH 7.6, 1 mM MgCl₂) and stored at 4° C. refrigerator which is stable up to one month.

The physisorbed GO.glucose.aptamer was prepared following a previously reported method [36]. Briefly, 400 μg/mL GO and 8 μM aptamer, were mixed and incubated overnight in a medium of 150 mM Buffer A (150 mM NaCl, 25 mM HEPES, pH 7.5, 1 mM MgCl₂). The following day, the mixture was washed 3 times with 150 mM Buffer A and 6 minutes of centrifugation at 8000 rpm. After the final wash, the GO-aptamer conjugate was redispersed in 150 mM Buffer A and stored at 4° up to one month.

HMN-GO. NA Fabrication

To fabricate the HMN-GO.NA patches, first 50 mg of MeHA, 1 mg of photoinitiator (2-hydroxy-4-2-hydroxy-2methylpropiophenome, refered to herein as PI), and 1 mg and N,N′-Methylenebisacrylamide (MBA) per patch were mixed. Then 10 mM Buffer A (10 mM NaCl, 25 mM HEPES, pH 7.6, 1 mM MgCl₂ in nuclease-free water) was added to achieve a concentration of 100 mg/mL MeHA. The solution was sonicated and mixed occasionally until the MeHA, PI, and MBA were completely dissolved, and a clear mixture was obtained.

To cast the first layer (needle layer) of HMN-GO.NA, 150 μL of the prepared 100 mg/ml MeHA with an equivalent volume of the desired GO.NA solutions were mixed and de-bubbled by being left covered for 15-20 minutes in the dark at an ambient condition. 300 μL of the MeHA-GO.NA mixture was deposited on a negative polydimethylsiloxane (PDMS) mold (Mioint, Singapore), and vacuumed for 90 seconds. The MeHA-GO.NA was dried for 5 hours at room temperature, followed by the addition of the second layer (base layer) containing 700 μL of 50 mg/mL MeHA. The patches were dried overnight in the dark at room temperature. The patches were removed from the molds and cured under UV light for 40 minutes with the needles facing up.

Chemical Characterization

The Fourier-transform infrared spectroscopy (FTIR, Bruker Hyperion 3000 FTIR Microscope) was conducted to study the chemical bonds for MeHA hydrogel and MeHA hydrogel-GO films. To prepare the MeHA hydrogel films, MeHA hydrogel mixture (containing 50 mg/mL MeHA +1 mg/mL of MBA and photoinitiator) was made, dried overnight, and crosslinked under UV for 40 minutes. The MeHA-GO hydrogel film was prepared by mixing 300 A of the MeHA mixture with an equal volume of 100 μg/mL GO followed by similar steps as making MeHA hydrogel film. The FTIR spectrum is recorded from 400-4000 cm⁻¹ and the peaks at 1600-1610 and 3300-3305 have been assigned to N-H bending and stretching, respectively.

Electron Microscopy Imaging

To visualize the HMN-GO.NA array, an HMN-GO.NA patch was prepared, coated with a 2 nm thick layer of gold and imaged using a Hitachi SU5000 FESEM. To visualize and analyze MeHA, MeHA-GO, and MeHA-GO.NA pore sizes, thin films were prepared and allowed to swell in water for 20 minutes. The films were then snap-frozen with liquid nitrogen and freeze-dried for 48 hours followed by being coated with a 2 nm thick layer of gold and imaged using a Hitachi SU5000 FESEM.

Swelling Studies

The prepared HMN, HMN-GO, and HMN-GO.NA patches were prepared and weighed (W₀). A 1.4 wt % agar gel was prepared and covered with a thin layer of parafilm. The patches were inserted into the agar through the parafilm for and incubated for 10 minutes. The patches were then removed and immediately weighed (W_{T}). To calculate the swelling ratio, the following formula was used:

$$Swelling ratio ( \% ) = \frac{{W}_{T} - {W}_{0}}{{W}_{0}} \times 100 \%$$

Mechanical Test

The mechanical strength of HMN, HMN-GO, and HMN-GO.NA patches were measured using Instron 5548 micro tester equipped with a 500N compression loading cell. For each test, the HMN patch was placed flat on its backside (tips facing upwards) on a compression platen. The distance between two platens was set to 1.5 mm. A vertical force was applied (at a constant speed of 0.5 mm/min) by the other platen. The compression loading cell capacity was set to 70 N. The load (force; N) and displacement (distance; mm) was recorded by the testing machine every 0.1 s to create the load-displacement curve.

NA Release Experiments

The following patches were prepared as mentioned above: HMN only, HMN mixed with 2 μM Cy3 ATP aptamer solution, HMN-GO.ATP.aptamer (the covalent form), and HMN-GO.ATP.aptamer (the physisorbed form). The release experiment was performed for three soaking times: 5 minutes, 10 minutes, and 30 minutes. Each patch was soaked in a solution consisting of 100 μL of 1× PBS with 4 mM ATP. After the soaking time, the patches were removed from their solutions. The solutions were diluted 1:10 using 1× PBS. 50 μL of the 1:10 solutions were aliquoted per well in a 96-well plate and fluorescence readings were performed. To account for any possible change in the fluorescent intensity caused by the HMN es, the fluorescent intensity obtained from all the conditioned was normalized to the fluorescent intensity obtained from HMN-only patches.

In-Vitro Testing

The HMN-GO.NA patches were prepared and imaged under the fluorescence microscope (Nikon, Ti2) with 10 ms exposure. 1.4 wt % agar gels containing the desired concentrations of the biomarkers of interest were prepared and covered with parafilm. The patches were applied on agar through the thin parafilm for 10 minutes, removed from the gel, and dried for 10 minutes under ambient conditions and at dark. Their fluorescence images were taken and the corresponding fluorescence response for each patch was calculated. The following buffers were used to prepare agar gels containing each biomarker of interest: Ringer's Buffer (147 mM NaCl, 4 mM KCl, 2.25 mM CaCl2, pH=7) for glucose, uric acid buffer (120 mM NaCl, 1 mM MgCl2, 20 mM Tris-HCl, 5 mM KCl, pH=7.4) for UA, serotonin buffer (1× PBS with 2 mM MgCl2, pH=7.4.) for serotonin, and insulin buffer (10 mM Buffer A (10 mM NaCl, 25 mM HEPES, pH 7.6, 1 mM MgCl2 in nuclease-free water) for insulin.

Ex-Vivo Testing

For the ex-vivo experiments, porcine skins (pig skin ears) were trimmed to 1 cm by 1 cm squares, rinsed with DI water, and equilibrated with different concentrations of the biomarkers of interest in their specific buffers. The skin-biomarker equilibration time for glucose, UA, and miR was 16 hours, but this equilibration time was 8 hours for serotonin and insulin due to their less stable nature. HMN-GO.NA patches were then prepared, and their baseline fluorescence intensity was recorded by the fluorescence microscope. The patches were then inserted into dried and biomarker-equilibrated porcine skin and fixed using Tegaderm tape. After 5 minutes, the patches were removed from the skin and left in dark under the ambient conditions to completely dry. The fluorescent intensity of patches after skin application was recorded based and patch responses were calculated based on which the associated calibration curves were drawn.

Specificity Test

For the biomarker specificity experiment, trimmed and washed porcine skins were equilibrated with a buffer (1× PBS, 2 mM KCl, 20 mM Tris-HCl, 2 mM MgCl₂ at pH 7.4) containing 10 mM glucose, 200 μM UA, 5 μM insulin, and 2 μM serotonin. For miR specificity experiments, the prepared porcine skins were equilibrated in buffer A containing 200 nM miR210 and miR21. HMN-GO.NA patches specific to each biomarker were fabricated as outlined before and their baseline fluorescence intensity was recorded by a fluorescence microscope. The patches were then applied to the porcine skins containing a mixture of different biomarkers, removed after 5 minutes, left to dry, and imaged with the fluorescence microscope. The obtained patch fluorescent responses were interpolated in the established ex-vivo calibration curves based on which the concentrations of captured biomarkers were calculated. The patch's percentage accuracies are defined as “[1−(calculated concentration of biomarker/actual concentration of biomarker present in skin)]*100%”.

H&E Staining

HMN-GO.NA patches were applied on the shaved dorsal rat skin for 5 minutes. The rat was euthanized 5 minutes (for cavity visualization) and 1 hour (for inflammatory marker visualization) after removing the HMN-GO.NA patches and the skin sections were cut and washed with 0.9% NaCl solution. The samples were fixed with neutral buffered 10% formalin for 24 hours, then stored in 70% ethanol and refrigerated. Fixed skin samples were cryopreserved in 15% sucrose/PBS solution at 4C overnight. Following cryopreservation, samples were briefly rinsed in PBS to remove residual sucrose. Samples were mounted on cork using OCT (TissueTek), frozen in isopentane cooled by liquid nitrogen, and stored at −80C. 10-micron sections were cut using a cryostat maintained at −20C and mounted on microscope slides. Hematoxylin and eosin (H&E) stains were used to identify the basic morphology of skin samples. Slides were stained with Harris-modified hematoxylin (Sigma, HHS32) for 30-seconds, washed in distilled water, and counterstained with 1% Eosin Y (Sigma, E4009) for 2 minutes. Slides were washed in distilled water, dehydrated in 75% and 95% ethanol, and cleared in Xylene prior to mounting with Permount mounting medium (Fisher Scientific, SF15). Images were acquired using a Cytation-5 multimode imager (Agilent). Images were obtained at 20× magnification and stitched together using the Gen5 software (Agilent).

In-Vivo Measurement of Glucose and Uric Acid

In-vivo experiments were done following the Guidelines for the Care and Use of Laboratory Animals and the Animal Welfare Act Regulations; all protocols were approved by the University of Waterloo Institutional Animal Care and Use Committee. To examine the performance of HMN-GO.glucose.aptamer and HMN-GO.UA.aptamer, an established model of streptozotocin (STZ)-induced diabetic rat was used. Male Sprague Dawley rats (Charles River, 100-150 gr) were injected with STZ (65 mg/kg) which destroys the host's pancreatic beta-cells secreting insulin. The STZ-injected rats were monitored for 1 week and their blood glucose was measured every 2 days using a glucose meter (OneTouchR UltraR, LifeScan, Inc., USA). Diabetic rats with blood sugar stabilized above 17 mM were selected for this study. Before applying the HMN-GO.glucose.aptamer patches, the rats fasted for 5 hours. The fasting blood and ISF glucose levels were measured by a glucometer and HMN-GO.glucose.aptamer, respectively. Subsequently, 4 units of insulin were injected into the rats and blood glucose levels were tracked by glucometer every 5 min. The HMN-GO.glucose.aptamer patches were applied to rat's skin and fixed with Tegaderm tape, when blood glucose levels reached certain ranges of >30 mM, 20-30 mM, 10-20 mM, and <10 mM. For two of the rats, subsequent to the final decrease in blood glucose, the rats were fed to increase blood glucose to about 20 mM, when another round of HMN-GO.glucopse.aptamer patches were applied (T4: 15-20 mM).

Totally, the blood and ISF glucose levels were tested at five-time points in each rat. The removed patches were dried at room temperature and used for post-insertion imaging.

HMN-GO.UA.aptamer patches were applied to ten individual male Sprague Dawley rats; three diabetic and seven healthy rats. The prepared HMN-GO.UA.aptamer patches were applied to the dorsal skin of rats for five minutes during which their blood was also collected from the tail vein using a blood collection system (Microvette® CB 300, Kent Scientific Corporation). The collected blood was then clotted by being left undisturbed at room temperature for 30 minutes, followed by centrifugation at 2000 g and at 4 degrees for 10 minutes. The resultant blood serum was collected and tested for uric acid concentration using the fluorometric uric acid assay kit (abcam, ab65344) following the product's instructions.

Rat skin was shaved, treated with hair removal cream, and cleaned prior to all HMN applications and the HMN was applied on rats for a total of five minutes. After the five minutes application, the removed patches were dried at room temperature and used for post-insertion imaging. To calculate the fluorescent response of patches, the HMN-GO.glucose.aptamer and HMN-GO.UA.aptamer patches were once imaged before skin application and once post-skin application. Rats' ISF UA and glucose levels were calculated by interpolating the fluorescent response into the ex vivo detection calibration curves.

Smartphone-Based Fluorescence Imaging Setup

The smartphone-based fluorescence imaging system was enclosed by a custom-made Blackbox with a provision to change the sample. The HMN-GO.NA was taped to the microscopic glass slide that was illuminated/excited by a 532 nm diode laser (10 mW) driven by a 3V battery. The fluorescence signal was collected through a 4× objective lens (Olympus, NA=0.10, WD=18.5 mm) and bandpass filter (Ø25 mm, CWL=570 nm, FWHM=10 nm). A 3D printed laser holder was used to maintain a low incident angle (˜10°) of the laser beam to the sample. The fluorescence image of the MN patch was taken by a Smartphone (Model-iPhone 13) and analyzed by ImageJ software.

Statistical Analysis

The statistical analysis was conducted using GraphPad Prism 9. The statistical difference between the swelling ratio of HMN, HMN-GO, and HMN-GO.NA was examined using the student T-test. Each experiment was performed in three replicates unless otherwise stated. All data are expressed as mean and the error bars represent the SEM. The calibration curves for both ex-vivo and in-vitro experiments were drawn based on patch fluorescent response. The response is defined as [F (fluorescent intensity after patch application)−F₀ (fluorescent intensity before patch application)]/F₀ (fluorescent intensity before patch application). The concentration of biomarkers for in-vivo experiments was determined by interpolating the obtained response in the ex-vivo calibration curves. The LOD is defined as the minimum target concentration that can be reliably detected by our HMN-GO.NA assay and is calculated using the method introduced by Armbruster et al⁷⁰.

TABLE 4
Nucleic acid sequences used in Example 2 with their modification, selected
from the literature references and ordered from IDT-Integrated DNA Technologies.

| | | Literature |
|---|---|---|
| Nucleic acid sequences | Biomarkers | Reference |
| /5Cy3/CTCTCGACGACATTACGGGACCTTGCTAAAGGTGGAATTATGTCGT/ | Uric Acid | (3) |
| 3AmMO/ (SEQ ID NO. 22) | (UA) | |
| /5Cy3/CTCTCGGGACGACCGTGTGTGTTGCTCTGTAACAGTGTCCATTGTCG | Glucose | (4) |
| TCCC (SEQ ID NO. 23) | | |
| /5AmMC6/GGTGGTGGGGGGGGTTGGTAGGGTGTCTTC/3Cy3/ | Insulin | (5, 6) |
| (SEQ ID NO. 24) | | |
| /5AmMC6/GACTGGTAGGCAGATAGGGGAAGCTGAT/iCy3/TCGATGCGTGG | Serotonin | (4) |
| GTC (SEQ ID NO. 25) | | |
| /5Cy3/CACCTGGGGGAGTATTGCGGAGGAAGG/3AmMO/ | Adenosine | (2) |
| (SEQ ID NO. 26) | three | |
| | phosphates | |
| | (ATP) | |
| pDNA | Biomarkers | |
| /5Cy3/TCAGCCGCTGTCACACGCACAG/3AmMO/ (SEQ ID NO. 27) | miR210 | |
| /5Cy3/TCAACATCAGTCTGATAAGCTA/3AmMO/ (SEQ ID NO. 28) | miR21 | |

Cy3-Cyanine 3 modification
5AmMC6 is 5′ Amino Modifier C6 modification
3AmMO is 3′ Amino Modifier modification

### Example 2

### Supplementary Information

Demonstrating Proof of Concept for HMN-GO.NA Using ATP Aptamer

Since ATP aptamer repetitively demonstrated desired binding affinity to its biomarker (adenosine three phosphates abbreviated as ATP) it was decided to first demonstrate the concept using this aptamer. Therefore, HMN-GO.ATP.aptamers were fabricated and applied on agarose gels containing different concentrations of ATP (FIG. 25A). The observed fluorescent response showed a strong linear correlation with ATP concentration (R²=0.984), indicating that the HMN-GO.ATP.aptamer could accurately detect ATP (FIG. 25A). Further, the HMN-GO.ATP aptamer showed a negligible response in contact with a non-specific target, supporting the specificity of HMN-GO.ATP.aptamer patches towards ATP (FIG. 25B). Next, the same concept was applied to detect other biomarkers with high clinical importance. See FIG. 26A-26F and FIG. 27A and FIG. 27B.

FIG. 25A and FIG. 25B shows In-vitro ATP detection to establish proof of concept. (A) Data representing the HMN-GO.ATP.aptamer response in the presence of different concentrations of its target (ATP) in agarose gel. A strong linear correlation with R²=0.984 was observed. At least three patches were tested per condition and the error bars represent standard error of the mean (SEM). (B) Bar graph showing the response of each HMN-GO.ATP.aptamer patch in agarose gels containing different concentrations of ATP and a non-specific target (10 μM insulin). Error bars represent the SEM.

FIG. 26A-26F shows in-vitro validation of HMN-GO.NA patch. Data representing the mean response of (A) HMN-GO.UA.aptamer, (B) HMN-GO.glucose.aptamer, (C) HMN-GO.insulin.aptamer, (D) HMN-GO.serotonin.aptamer, (E) HMN-GO.pDNA210, and (F) HMN-GO.pDNA21 in the presence of different concentrations of their targets in agarose gel. At least three patches were tested per condition and error bars represent SEM.

FIG. 27A and FIG. 27B shows histology imaging analysis of (A) a control rat's skin (scale bar=2400 μm) and (B) rat's skin one hour after HMN-GO.NA removal (scale bar=2000 μm).

### Example 2

### References

2. B. D. Wilson, A. A. Hariri, I. A. P. Thompson, M. Eisenstein, H. T. Soh, Independent control of the thermodynamic and kinetic properties of aptamer switches. Nat Commun. 10, 1-9 (2019).
3. Y. Liu, J. Liu, Selection of DNA Aptamers for Sensing Uric Acid in Simulated Tears. Analysis & Sensing (2022), doi:10.1002/anse.202200010.
4. N. Nakatsuka, K. A. Yang, J. M. Abendroth, K. M. Cheung, X. Xu, H. Yang, C. Zhao, B. Zhu, Y. S. Rim, Y. Yang, P. S. Weiss, M. N. Stojanović, A. M. Andrews, Aptamer-field-effect transistors overcome Debye length limitations for small-molecule sensing. Science (1979). 362, 319-324 (2018).
5. W. Yoshida, E. Mochizuki, M. Takase, H. Hasegawa, Y. Morita, H. Yamazaki, K. Sode, K. Ikebukuro, Selection of DNA aptamers against insulin and construction of an aptameric enzyme subunit for insulin sensing. Biosens Bioelectron. 24, 1116-1120 (2009).
6. Y. Wu, B. Midinov, R. J. White, Electrochemical aptamer-based sensor for real-Time monitoring of insulin. ACS Sens. 4, 498-503 (2019).
9. Yang, J. et al. Recent Progress in Microneedles-Mediated Diagnosis, Therapy, and Theranostic Systems. Adv Healthc Mater (2022) doi:10.1002/adhm.202102547.
18. Gao, J., Huang, W., Chen, Z., Yi, C. & Jiang, L. Simultaneous detection of glucose, uric acid and cholesterol using flexible microneedle electrode array-based biosensor and multi-channel portable electrochemical analyzer. Sens Actuators B Chem 287, 102-110 (2019).
24. Liu, Z. et al. Intracellular detection of ATP using an aptamer beacon covalently linked to graphene oxide resisting nonspecific probe displacement. Anal Chem 86, 12229-12235 (2014).
36. Wu, M., Kempaiah, R., Huang, P. J. J., Maheshwari, V. & Liu, J. Adsorption and desorption of DNA on graphene oxide studied by fluorescently labeled oligonucleotides. Langmuir 27, 2731-2738 (2011).60. Kuo, C. S. et al. Portable electrochemical blood uric acid meter. J Clin Lab Anal 16, 109-114 (2002).
62. Cho, S. K., Chang, Y., Kim, I. & Ryu, S. U-Shaped Association Between Serum Uric Acid Level and Risk of Mortality: A Cohort Study. Arthritis and Rheumatology 70, 1122-1132 (2018).
70. Armbruster, D. A. & Pry, T. Limit of blank, limit of detection and limit of quantitation. Clin Biochem Rev 29 Suppl 1, S49-52 (2008).

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed is:

1. A microneedle for detecting a target, the microneedle comprising:
a hydrogel;
a probe coupled to the hydrogel, the probe for generating a measurable signal in the presence of the target.

2. The microneedle of claim 1, wherein the hydrogel comprises a polymer comprising at least one C═C functionality; an acrylated polymer, a methacrylated polymer, or a combination thereof; and/or methacrylated gelatin, methacrylated hyaluronic acid, methacrylated alginate, methacrylated chitosan, methacrylated collagen, methacrylated polyethylene glycol, methacrylated polyvinyl alcohol, methacrylated polylysine, or a combination thereof.

3. The microneedle of claim 1, wherein the hydrogel further comprises a conductive polymer, an ionomer, or a combination thereof; or poly(3,4-ethylenedioxythiophene) polystyrene sulfonate; polyacetylene; polypyrrole; polyindole; polyaniline; a copolymer thereof;
or a combination thereof.

4. The microneedle of claim 1, wherein the probe coupled to the hydrogel comprises the probe being coupled to the hydrogel by covalent bonding, intermolecular bonding, physisorption, complexation, a linker; or a combination thereof.

5. The microneedle of claim 1, wherein the probe comprises a nucleic acid, wherein the nucleic acid is an nucleic acid that binds to the target; and wherein the nucleic acid optionally comprises an aptamer, single stranded complementary probe DNA, peptide nucleic acid, nucleic acid enzyme, or combinations thereof.

6. The microneedle of claim 5, wherein the nucleic acid comprises a linker functional group for coupling the probe to the hydrogel.

7. The microneedle of claim 6, wherein the linker functional group comprises a phosphoramidite functional group; or an acrydite functional group.

8. The microneedle of claim 1, wherein the probe comprises a fluorophore; or an electroactive species, a redox active species, or a combination thereof.

9. The microneedle of claim 1, wherein the probe comprises a nucleic acid, and the nucleic acid comprises a fluorophore or is linked to a fluorophore, or the nucleic acid comprises a redox reporter or is linked to a redox reporter.

10. The microneedle of claim 1, wherein the probe further comprises a quencher, and the probe is optionally reversibly bound to a quencher, or is optionally tethered to the quencher via covalent bonding, intermolecular bonding, physical adsorption, conjugation, or a combination thereof.

11. The microneedle of claim 10, wherein the probe comprises a nucleic acid and the quencher comprises a sequence partially or fully complimentary to at least a portion of the nucleic acid sequence.

12. The microneedle of claim 10, wherein the probe comprises
a nucleic acid and the quencher comprises a graphene-based material, wherein the graphene-based material optionally comprises graphene-oxide (GO) nanosheets, graphene-oxide (GO) nanoparticles, graphene-oxide (GO) nanocomposites, or a combination thereof.

13. The microneedle of claim 1, wherein the measurable signal is fluorescence; or an electrochemical signal.

14. The microneedle of claim 1, wherein the target comprises a biomolecule present in interstitial fluid.

15. The microneedle of claim 14, wherein the target comprises small biomolecules, proteins, or micro ribonucleic acids; or cortisol, vanomycin, gentamicin, tyrosinamide, thrombin, micro-RNA miR21, micro-RNA miR210, uric acid (UA), serotonin, insulin, adenosine triphosphate, or glucose.

16. The microneedle of claim 1, wherein the microneedle has a length of about 300 μm to about 1000 μm, such as about 800 μm.

17. The microneedle of claim 1, further comprising a conductive material, wherein the conductive material optionally comprises a metal nanoparticle, graphene-based material, conductive polymer, or an ionomer, or a combination thereof.

18. A method of producing a microneedle, the method comprising:
combining a functionalized hydrogel, a probe precursor, optionally a conductive material, and a crosslinking agent in a mold; and
exposing the mixture in the mold to UV light to link at least a portion of the probe to the functionalized hydrogel and to form a crosslinked material..

19. The method of claim 18, further comprising:
removing the crosslinked material from the mold; and
further exposing the unmolded crosslinked material to UV light.

20. The method of claim 18, wherein combining the functionalized hydrogel, the probe precursor, optionally a conductive material, and the crosslinking agent in the mold comprises:
dissolving about 50:1 to about 10:1 (wt/wt) of functionalized hydrogel:crosslinking agent in a buffer to form a functionalized hydrogel solution;
optionally adding a conductive material to the functionalized hydrogel solution;
optionally degassing the functionalized hydrogel solution;
adding the functionalized hydrogel solution to the mold;
partially drying the functionalized hydrogel solution in the mold;
optionally, adding further functionalized hydrogel solution to the mold;
adding the probe precursor to the mold; and
optionally, drying the mixture in the mold further.

21. The method of claim 18, wherein the probe precursor comprises a solution of nucleic acid and optionally a quencher.

22. The method of claim 18, wherein exposing the mixture in the mold to UV light comprises exposing the mixture to light of about 200 nm to about 400 nm, preferably about 360 nm light;
and preferably for about 1 min to about 1 hour, such as about 10 to about 20 min.

23. The method of claim 18, wherein the hydrogel comprises hyaluronic acid; and
functionalizing functionalizing the hydrogel comprises reacting hyaluronic acid with methacrylic anhydride to form methacrylated hyaluronic acid.

24. The method of claim 18, wherein the optional conductive material comprises metal nanoparticles, graphene-based material, or a conductive polymer or ionomer, such as poly(3,4-ethylenedioxythiophene) polystyrene sulfonate, polyacetylene, polypyrrole, polyindole, polyaniline, or copolymers thereof.

25. The method of claim 18, wherein the mold is a negative polydimethylsiloxane mold.

26. An apparatus for detecting a target in a sample, the apparatus comprising:
the microneedle according to claim 1; and
a detector for detecting the measurable signal.

27. A transdermal patch comprising the microneedle according to claim 1.

28. A method for transdermal biosensing of a target in a subject, the method comprising:
applying the transdermal patch according to claim 27;
detecting the measurable signal; and
associating the measurable signal to the concentration of the target in the subject.

29. The method of claim 28, wherein detecting the measurable signal is reagentless.

30. The method of claim 28, wherein detecting the measurable signal comprises measuring the fluorescence intensity of the probe; or measuring an electrochemical signal.

31. The method of claim 28, wherein associating the measurable signal comprises comparing a measured intensity of the measurable signal to a calibration curve of measured intensities of known concentrations of the target.


SEQUENCE LISTING (28 sequences)

SEQ ID NO: 1 - DNA, 30 bp, synthetic construct
  source 1..30: mol_type = other DNA
ggtggtggggggggttggtagggtgtcttc

SEQ ID NO: 2 - DNA, 27 bp, synthetic construct
  source 1..27: mol_type = other DNA
aatgcggggtggagaatggttgccgca

SEQ ID NO: 3 - DNA, 66 bp, synthetic construct
  source 1..66: mol_type = other DNA
ctctcgggacgaccgagggtaccgcaatagtacttattgttcgcctattgtgggtcgggtcgtccc

SEQ ID NO: 4 - DNA, 26 bp, synthetic construct
  source 1..26: mol_type = other DNA
gggacttggtttaggtaatgagtccc

SEQ ID NO: 5 - DNA, 51 bp, synthetic construct
  source 1..51: mol_type = other DNA
ctctcgggacgaccgtgtgtgttgctctgtaacagtgtccattgtcgtccc

SEQ ID NO: 6 - DNA, 27 bp, synthetic construct
  source 1..27: mol_type = other DNA
cacctgggggagtattgcggaggaagg

SEQ ID NO: 7 - DNA, 37 bp, synthetic construct
  source 1..37: mol_type = other DNA
ggagcttggattgatgtggtgtgtgagtgcggtgccc

SEQ ID NO: 8 - DNA, 15 bp, synthetic construct
  source 1..15: mol_type = other DNA
ggttggtgtggttgg

SEQ ID NO: 9 - DNA, 42 bp, synthetic construct
  source 1..42: mol_type = other DNA
gactggtaggcagataggggaagctgattcgatgcgtgggtc

SEQ ID NO: 10 - DNA, 46 bp, synthetic construct
  source 1..46: mol_type = other DNA
ctctcgacgacattacgggaccttgctaaaggtggaattatgtcgt

SEQ ID NO: 11 - DNA, 22 bp, synthetic construct
  source 1..22: mol_type = other DNA
  misc_feature 1: note = Cyanine 3
  misc_feature 22: note = Amino Modifier
tcagccgctgtcacacgcacag

SEQ ID NO: 12 - DNA, 22 bp, synthetic construct
  source 1..22: mol_type = other DNA
  misc_feature 1: note = Cyanine 3
  misc_feature 22: note = Amino Modifier
tcaacatcagtctgataagcta

SEQ ID NO: 13 - DNA, 51 bp, synthetic construct; identical to SEQ ID NO: 5
  source 1..51: mol_type = other DNA
  misc_feature 1: note = Acrydite and Cyanine 3

SEQ ID NO: 14 - DNA, 14 bp, synthetic construct
  source 1..14: mol_type = other DNA
  misc_feature 14: note = Dabcyl
ggtcgtcccgagag

SEQ ID NO: 15 - DNA, 27 bp, synthetic construct
  source 1..27: mol_type = other DNA
  misc_feature 1: note = Acrydite and Cyanine 3
cacctgggggagtattgcggaggaagg

SEQ ID NO: 16 - DNA, 11 bp, synthetic construct
  source 1..11: mol_type = other DNA
  misc_feature 11: note = Dabcyl
ttttccaggtg

SEQ ID NO: 17 - DNA, 37 bp, synthetic construct; identical to SEQ ID NO: 7
  source 1..37: mol_type = other DNA
  misc_feature 1: note = Acrydite and Cyanine 3

SEQ ID NO: 18 - DNA, 10 bp, synthetic construct
  misc_feature 10: note = Dabcyl
  source 1..10: mol_type = other DNA
tcacatcaat

SEQ ID NO: 19 - DNA, 20 bp, synthetic construct
  source 1..20: mol_type = other DNA
  misc_feature 1: note = Acrydite and Cyanine 3
  misc_feature 15: note = Dabcyl
  misc_feature 5^6: note = Internal Spacer 18 - (CH2CH2O)30
ccaacggttggtgtggttgg

SEQ ID NO: 20 - DNA, 15 bp, synthetic construct
  source 1..15: mol_type = other DNA
  misc_feature 1: note = Acrydite and Cyanine 3
ggttggtgtggttgg

SEQ ID NO: 21 - DNA, 10 bp, synthetic construct
  source 1..10: mol_type = other DNA
  misc_feature 10: note = Dabcyl
tttttcaacc

SEQ ID NO: 22 - DNA, 46 bp, synthetic construct; identical to SEQ ID NO: 10
  source 1..46: mol_type = other DNA
  misc_feature 1: note = Cyanine 3
  misc_feature 46: note = Amino Modifier

SEQ ID NO: 23 - DNA, 51 bp, synthetic construct; identical to SEQ ID NO: 5
  source 1..51: mol_type = other DNA
  misc_feature 1: note = Cyanine 3

SEQ ID NO: 24 - DNA, 30 bp, synthetic construct; identical to SEQ ID NO: 1
  source 1..30: mol_type = other DNA
  misc_feature 30: note = Cyanine 3
  misc_feature 1: note = Amino Modifier C6

SEQ ID NO: 25 - DNA, 42 bp, synthetic construct; identical to SEQ ID NO: 9
  source 1..42: mol_type = other DNA
  misc_feature 28^29: note = Internal Cyanine 3
  misc_feature 1: note = Amino Modifier C6

SEQ ID NO: 26 - DNA, 27 bp, synthetic construct
  source 1..27: mol_type = other DNA
  misc_feature 1: note = Cyanine 3
  misc_feature 27: note = Amino Modifier
cacctgggggagtattgcggaggaagg

SEQ ID NO: 27 - DNA, 22 bp, synthetic construct
  source 1..22: mol_type = other DNA
  misc_feature 1: note = Cyanine 3
  misc_feature 22: note = Amino Modifier
tcagccgctgtcacacgcacag

SEQ ID NO: 28 - DNA, 22 bp, synthetic construct
  source 1..22: mol_type = other DNA
  misc_feature 1: note = Cyanine 3
  misc_feature 22: note = Amino Modifier
tcaacatcagtctgataagcta